(19) United States
(12) Patent Application Publication
(10) Pub. No.: US 2026/0317392 A1
(43) Pub. Date: Oct. 15, 2026

(54) LONG-ACTING GROWTH HORMONE RECEPTOR ANTAGONIST AND USE THEREOF

(71) Applicant: Novo Nordisk A/S, Bagsvaerd (DK)

(72) Inventors: Carsten Behrens, Koebenhavn N (DK); Peter Thygesen, Koebenhavn Oe (DK)

(21) Appl. No.: 19/480,529

(22) PCT Filed: May 8, 2024
(86) PCT No.: PCT/EP2024/062796

§ 371 Date: Oct. 31, 2025

(30) Foreign Application Priority Data
May 12, 2023 (EP) 23173001.1

(51) Int. Cl. C07K 14/61; A61K 38/00; A61P 5/00

(52) U.S. Cl. CPC C07K 14/61; A61P 5/00; A61K 38/00; C07K 2319/70

(57) ABSTRACT

The invention relates to a long-acting growth hormone receptor antagonist comprising a growth hormone variant and an albumin binding moiety. Further described are methods of preparing said compound and use thereof in medicine e.g., for the treatment of acromegaly.

### TECHNICAL FIELD

The present invention relates to a compound that is a long-acting growth hormone receptor antagonist comprising a growth hormone variant and an albumin binding moiety, to methods of preparing said compound, and the use of said compound in medicine.

### BACKGROUND

Growth hormone (GH) is a polypeptide secreted by the anterior pituitary in mammals. Dependent on species growth hormone is a protein composed of approximately 190 amino acid residues corresponding to a molecular weight of approximately 22 kDa. Human growth hormone (hGH) is composed of 191 amino acids according to SEQ ID NO: 1. Growth hormone binds to and signals through cell surface receptors, the growth hormone receptors (GHR). Growth hormone plays a key role in promoting growth, maintaining normal body composition, anabolism, and lipid metabolism. When the body excretes excessive amounts of GH, plasma levels of Insulin-like Growth Factor 1 (IGF-1) are elevated causing body tissue and bones to grow more quickly resulting in diseases such as acromegaly.

Acromegaly is usually diagnosed in adults aged 30-50 years and the underlying cause is in most cases due to a slowly growing pituitary adenoma. First line of treatment is surgery. However, if surgery is not possible or fails to achieve adequate control of hGH and IGF-1 levels, medical treatment with somatostatin analogues or recombinant hGH receptor antagonists is next level of treatment.

Pegvisomant was first described in WO1997/011178 and is a PEGylated version of the potent GH receptor antagonist B2036 (SEQ ID NO: 5) and has been marketed as Somavert® for more than 20 years for the treatment of acromegaly. Pegvisomant is a competitive hGH antagonist consisting of a peptide backbone, B2036, decorated with a variation of 4-6 PEG chains and a variation in pegylation positions via lysines at positions F1, K145, K140, K38, K158, K120, K70 (Pradhananga et al., J Mol Endo (2002), 29, 11-14; U.S. Pat. No. 5,849,535). However, it is associated with several shortcomings such as injection site reactions, immunogenicity, painful injections and being a heterogeneous product make isolation complicated. Also, Pegvisomant is obtained as a solid that is reconstituted in buffer prior to injection, and it is known to partly aggregate when reconstituted, and due to high viscosity of the reconstituted product there is a need for using a large needle (271/2G) for subcutaneous injection causing discomfort to the patients. To obtain a sustainable IGF-1 reduction, Pegvisomant is administered once daily. However, if used in combination with a somatostatin analogue the administration frequency may be reduced to 2-3 administrations per week.

Several attempts have been made to overcome these shortcomings. E.g., alternatively PEGylated growth hormone receptor antagonists have been described in e.g., CN103087182B and U.S. Pat. No. 10,874,717, and alternative strategies for improving half-life and/or providing backbone substitutions of growth hormone receptor antagonists have been described in e.g., US2020/03999340 using alpha-1 antitrypsin fusions, WO2019/211842 using carboxy-terminal peptide conjugates and J. Biol. Chem. 2021, 296, 100588 suggesting specific backbone substitutions.

Hence, there is still a need for the provision of growth hormone receptor antagonists with improved properties, such as e.g., improved binding affinity and/or prolonged half-life. The present invention provides a novel long-acting growth hormone receptor antagonist with improved pharmacological, pharmacokinetic properties as well as providing improved convenience for the patient.

### SUMMARY

The invention relates to a long-acting growth hormone receptor antagonist (LA-GHRA) comprising a growth hormone variant and an albumin binding moiety. In some aspects, the growth hormone variant is a polypeptide derived from human growth hormone (hGH) and comprises amino acid substitutions H18D, H21N, L101C, G120R/G120K, R167N, D171S, E174S, 1179T compared to hGH (SEQ ID NO: 1). In one embodiment, the albumin binding moiety is attached to the growth hormone variant via the sulphur on the side chain of the cysteine at position 101 of the polypeptide.

In one aspect, a long-acting growth hormone receptor antagonist is provided comprising:
a. a growth hormone variant comprising at least the following amino acid substitutions as compared to human growth hormone (hGH) (SEQ ID NO: 1): L101C, G120R/G120K, and wherein the growth hormone variant has at least 80% sequence identity to the polypeptide of SEQ ID NO: 1; and
b. an albumin binding moiety configured for binding to albumin, for example binding to albumin with a dissociation constant (Kd) of less than 1 μM, wherein the albumin binding moiety has a molecular weight of 3 kDa or less and is covalently attached to the growth hormone variant via the sulphur residue of the cysteine side chain at position 101 of the growth hormone variant;

or a pharmaceutically acceptable salt thereof.

In one aspect, a long-acting growth hormone receptor antagonist is provided comprising:
a. a growth hormone variant comprising the following amino acid substitutions as compared to human growth hormone (hGH) (SEQ ID NO: 1): H18D, H21N, L101C, G120R/G120K, R167N, D171S, E174S, 1179T, and
b. an albumin binding moiety defined by Chem. 2

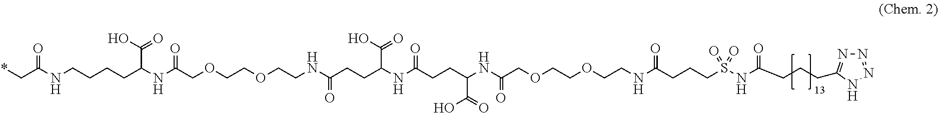

wherein * denotes the attachment point of the albumin binding moiety to the growth hormone variant via the sulphur residue of the cysteine side chain at position 101 of the growth hormone variant; or a pharmaceutically acceptable salt thereof.

In one aspect, a long-acting growth hormone receptor antagonist is provided of compound 1a:

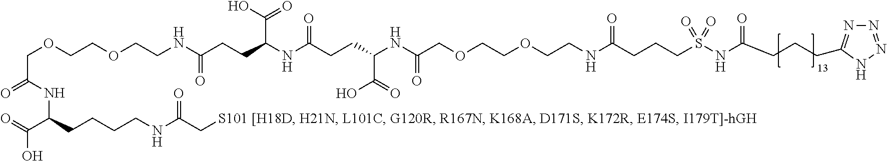

(compound 1a), or a pharmaceutically acceptable salt thereof, optionally having one or two disulphide bridges between Cys53-Cys165 and/or Cys182-Cys189.

In one aspect, a long-acting growth hormone receptor antagonist is provided of compound 1a:

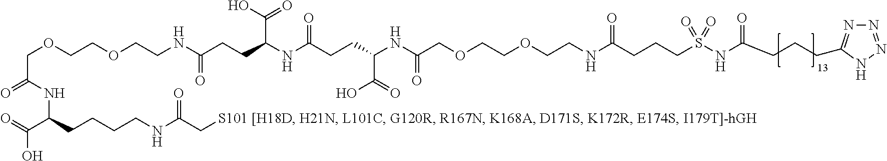

(compound 1a), or a pharmaceutically acceptable salt thereof.

The invention further relates to a pharmaceutical composition comprising said LA-GHRA and pharmaceutically acceptable excipients, the medical use of said antagonist, as well as a nucleic acid encoding the polypeptide backbone, a vector comprising said nucleic acid and a host cell comprising said vector.

In one aspect, a long-acting growth hormone receptor antagonist is provided as defined herein for use in the treatment or prevention of diseases caused by excess human growth hormone and/or IGF-1 levels, such as acromegaly or gigantism.

In one aspect, a nucleic acid encoding a human growth hormone variant is provided comprising the following amino acids modifications compared to hGH:
i) H18D, H21N, L101C, G120R, R167N, K168A, D171S, K172R, E174S, 1179T (GHv-1);
ii) H18D, H21N, L101C, G120R, N149D, N152D, R167N, K168A, D171S, K172R, E174S, 1179T (GHv-2); or
iii) H18D, H21N, L101C, G120R, N149D, N152D, R167N, D171S, E174S, 1179T (GHv-3).

In one aspect, a nucleic acid is provided encoding a human growth hormone variant comprising the following amino acids modifications compared to hGH:
H18D, H21N, L101C, G120R, R167N, K168A, D171S, K172R, E174S, 1179T.

In one aspect, a vector is provided comprising a nucleic acid encoding a human growth hormone variant comprising the following amino acid modification compared to hGH:
i) H18D, H21N, L101C, G120R, R167N, K168A, D171S, K172R, E174S, 1179T (GHv-1);
ii) H18D, H21N, L101C, G120R, N149D, N152D, R167N, K168A, D171S, K172R, E174S, 1179T (GHv-2); or
iii) H18D, H21N, L101C, G120R, N149D, N152D, R167N, D171S, E174S, 1179T (GHv-3).

In one aspect, the invention provides a LA-GHRA that binds to the hGHR with improved binding affinity compared to pegvisomant.

Also, or alternatively, in a second aspect, the invention provides a LA-GHRA that is an antagonist at the hGHR, thus, inhibiting or reducing the natural response that hGH exerts on the hGHR and shows improved antagonistic potency as compared to pegvisomant.

Also, or alternatively, in a third aspect, the invention provides a LA-GHRA that shows improved pharmacokinetic properties. In one aspect, the present disclosure provides a LA-GHRA that possess all of the above-described effects.

In one aspect, the present disclosure provides a process for preparing a human growth hormone variant as disclosed herein comprising culturing a host cell comprising a vector according to the present disclosure.

### BRIEF DESCRIPTION OF DRAWINGS

FIG. 1 shows plasma concentration-time profiles of Compound 1a after i.v. and s.c. administration.

### DESCRIPTION

### Definitions

In what follows, Greek letters may be represented by their symbol or the corresponding written name, for example: α=alpha; β=beta; ε=epsilon; γ=gamma; ω=omega; etc. Also, the Greek letter of μ may be represented by “u”, e.g. in μl=ul, or in μM=uM.

The terms “a” and “an” and “the” and similar referents as used in the context of de-scribing the invention are to be construed to cover both the singular and the plural (i.e. one or more), unless otherwise indicated herein or clearly contradicted by context.

Also described herein are growth hormone receptor antagonists, pharmaceutical compositions and uses thereof in which open ended terms like “comprises” and “comprising” are replaced with closed terms such as “consists of”, “consisting of”, and the like.

The invention relates to a long-acting growth hormone receptor antagonist (LA-GHRA) comprising a growth hormone variant and an albumin binding moiety.

The term “hGH” refers to human growth hormone, which is a 191 amino acid polypeptide with the sequence:

FPTIPLSRLFDNAMLRAHRLHQLAFDTYQEFEEAYIPKEQKYSFLQNPQTSLCFSES IPTPSNREETQQKSNLELLRISLLLIQSWLEPVQFLRSVFANSLVYGASDSNVYDLLKDLEEGI QTLMGRLEDGSPRTGQIFKQTYSKFDTNSHNDDALLKNYGLLYCFRKDMDKVETFLRIVQC RSVEGSCGF (SEQ ID NO: 1), which is the natural ligand for the human growth hormone receptor or “hGHR”. SEQ ID NO: 1 is also the sequence of recombinant human growth hormone also known as somatropin.

A receptor antagonist may be defined as a compound that binds to a receptor and is capable of inhibiting or reducing a response typical of the natural ligand. As described herein, a “growth hormone receptor antagonist” or “GHRA” refers to a compound capable of binding to the human growth hormone receptor without activating the growth hormone receptor or reducing the activation compared to the natural response of hGH.

In one embodiment, the growth hormone receptor antagonist is a growth hormone variant. The term “growth hormone variant” (GHv) refers to a hGH polypeptide comprising one or more substitutions as compared to hGH.

As used herein, the term “long-acting growth hormone receptor antagonist” or “LA-GHRA” refers to a chemically modified growth hormone receptor antagonist, in which an albumin binding moiety has been covalently attached to the polypeptide backbone. In one embodiment, LA-GHRA refers to a chemically modified growth hormone variant, in which an albumin binding moiety has been covalently attached to the polypeptide backbone. As used herein, an “albumin binding moiety” means a chemical moiety or group replacing a hydrogen atom.

As used herein, the term “sequence identity” refers to the degree of similarity between two polypeptide sequences, expressed as a percentage (%). The sequence identity can be determined by e.g. alignment, for instance based on a Needleman-Wunsch algorithm. This algorithm is described in Needleman, S. B. and Wunsch, C. D., (1970), Journal of Molecular Biology, 48:443-453, and the align program by Myers and W. Miller in “Optimal Alignments in Linear Space” CABIOS (computer applications in the biosciences) (1988) 4:11-17. The term “sequence identity” is used for example in some embodiments to describe a growth hormone variant as part of a LA-GHRA having at least 81% sequence identity to the polypeptide of SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to the polypeptide of SEQ ID NO: 1.

### Growth Hormone Variants

In the following, embodiments of growth hormone variants are disclosed. In particular embodiments, these are to be understood as specifically disclosed as part of a long-acting growth hormone receptor antagonist.

In one embodiment, the growth hormone variant comprises substitutions G120R/G120K and L101C, preferably G120R and L101C. In a further embodiment, the growth hormone variant further comprises one or more of the following substitutions as compared to hGH: H18D, H21N, R167N, D171S, E174S, and 1179T. In one embodiment, the growth hormone variant comprises the following substitutions as compared to hGH: H18D, H21N, L101C, G120R, R167N, D171S, E174S, and 1179T. In a particular embodiment, the growth hormone variant comprises the following substitutions as compared to hGH: H18D, H21N, L101C, G120R, R167N, K168A, D171S, K172R, E174S, and 1179T. In one embodiment, the growth hormone variant comprises substitutions H18D, H21N, L101C, G120R, R167N, K168A, D171S, K172R, E174S, and 1179T, and 1-10 further substitutions as compared to hGH, such as 1-5 further substitutions or 2-4 further substitutions. In a further embodiment, the growth hormone variant is: FPTIPLSRLFDNAMLRADRLNQLAFDTYQEFEEAYIPKEQKYSFLQNPQTSLCFSESIPTPSN REETQQKSNLELLRISLLLIQSWLEPVQFLRSVFANSCVYGASDSNVYDLLKDLEERIQTLMG RLEDGSPRTGQIFKQTYSKFDTNSHNDDALLKNYGLLYCFNADMSRVSTFLRTVQCRSVEG SCGF (SEQ ID NO: 2), which may also be referred to as [H18D, H21N, L101C, G120R, R167N, K168A, D171S, K172R, E174S, 1179T]-hGH or compound 2 or GHv-1. In one embodiment, SEQ ID NO: 2 comprises one or more disulphide bridge between Cys53-Cys165 and/or Cys182-Cys189.

In a further embodiment the growth hormone variant comprises substitutions H18D, H21N, L101C, G120R, N149D, N152D, R167N, K168A, D171S, K172R, E174S and |179T and 1-10 further substitutions as compared to hGH, such as 1-5 further substitutions or 2-4 further substitutions. In a further embodiment, the growth hormone variant is:

(GHv-2).
(SEQ ID NO: 3)
FPTIPLSRLFDNAMLRADRLNQLAFDTYQEFEEAYIPKEQKYSFLQNPQT
SLCFSESIPTPSNREETQQKSNLELLRISLLLIQSWLEPVQFLRSVFANS
CVYGASDSNVYDLLKDLEERIQTLMGRLEDGSPRTGQIFKQTYSKFDTDS
HDDDALLKNYGLLYCFNADMSRVSTFLRTVQCRSVEGSCGF.

In a further embodiment the growth hormone variant comprises substitutions H18D, H21N, L101C, G120R, N149D, N152D, R167N, D171S, E174S and 1179T and 1-10 further substitutions as compared to hGH, such as 1-5 further substitutions or 2-4 further substitutions. In a further embodiment, the growth hormone variant is:

(GHv-3).
(SEQ ID NO: 4)
FPTIPLSRLFDNAMLRADRLNQLAFDTYQEFEEAYIPKEQKYSFLQNPQT
SLCFSESIPTPSNREETQQKSNLELLRISLLLIQSWLEPVQFLRSVFANS
CVYGASDSNVYDLLKDLEERIQTLMGRLEDGSPRTGQIFKQTYSKFDTDS
HDDDALLKNYGLLYCFNKDMSKVSTFLRTVQCRSVEGSCGF.

In one embodiment, the growth hormone variant has at least 81% sequence identity to the polypeptide of SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to the polypeptide of SEQ ID NO: 1.

In one embodiment, the growth hormone variant further comprises one or more mutations selected from the group consisting of: H18D, H21N, N149D, N152D, R167N, K168A, D171S, K172R, E174S, and 1179T.

In one embodiment, the growth hormone variant comprises the following amino acid modifications compared to hGH:
H18D, H21N, L101C, G120R, R167N, K168A, D171S, K172R, E174S, and 1179T (GHv-1);
H18D, H21N, L101C, G120R, N149D, N152D, R167N, K168A, D171S, K172R, E174S, and 1179T (GHv-2); or
H18D, H21N, L101C, G120R, N149D, N152D, R167N, D171S, E174S, and |179T (GHv-3).

In one embodiment, the growth hormone variant in addition to L101C, G120R/G120K comprises 1-10 further amino acid substitutions, such as 2-8 or 3-5 further amino acid substitutions.

In one embodiment, the substitution at position 120 of the growth hormone variant is G120R.

In one embodiment, the growth hormone variant is 191 amino acids long.

In one embodiment, the growth hormone variant is a growth hormone receptor antagonist. In one embodiment, the growth hormone variant is capable of binding to the growth hormone receptor.

### Albumin Binders

In one aspect, the LA-GHRA comprises an albumin binding moiety covalently attached to the growth hormone variant via a cysteine, e.g., via the cysteine at position 101. The terms “albumin binder” and “albumin binding moiety” are used interchangeably herein. In a further aspect, the albumin binding moiety can form non-covalent conjugates with proteins, e.g., albumin, thereby promoting the circulation of the LA-GHRA with the blood stream, and also having the effect of protracting the time of action of the LA-GHRA, due to the fact that the conjugate of the LA-GHRA and albumin is only slowly disintegrated to release the active pharmaceutical ingredient.

In one embodiment, the albumin binding moiety has a molecular weight of 2.8 kDa or less, such as 2.6 kDa or less, such as 2.4 kDa or less, such as 2.2 kDa or less, such as 2.0 kDa or less, such as 1.8 kDa or less, such as 1.6 kDa or less, such as 1.4 kDa or less, such as 1.2 kDa or less, for example 1.0 kDa or less.

In one embodiment, the albumin binding moiety has a molecular weight of 0.3 kDa or more, for example 1.8 kDa or less but more than 0.3 kDa.

In one aspect, the albumin binding moiety is covalently attached to the growth hormone variant via a sulfhydryl group on the side chain of a backbone cysteine of the growth hormone variant. The albumin binding moiety may be attached to the growth hormone variant by reaction of the reduced cysteine side chain with an activated derivative of the albumin binding moiety comprising a leaving group, such as a haloacetamide as described on page 77, lines 5-19, Chemistry III, of WO2011/089255 or as Chem. 4a or Chem. 5a in example 1 herein. In one embodiment, the haloacetamide is selected from chloro-, bromo- and iodoacetamides Chem. 3, Chem. 4 and Chem. 5, respectively.

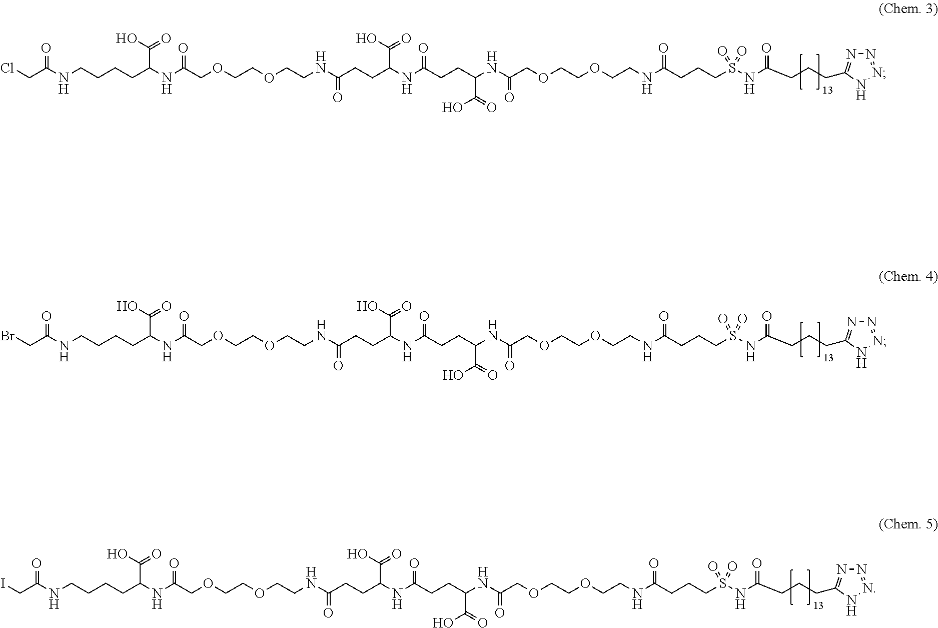

The structures of the “albumin binders” or “albumin binding moieties” when specified as part of the compounds of the present invention are the substitution products resulting from displacement of the leaving group e.g. chloro-, bromo- or iodo- of the haloacetamides depicted herein by the sulphur of position 101 of the growth hormone variants. The resulting albumin binder structure can readily be deduced from the final structure of the compounds disclosed herein in view of how the compounds are formed e.g. by displacement of a halogen leaving group from the depicted haloacetamides as demonstrates in the examples, e.g. Example 1 and Example 11.

In a further aspect the albumin binder is selected from:

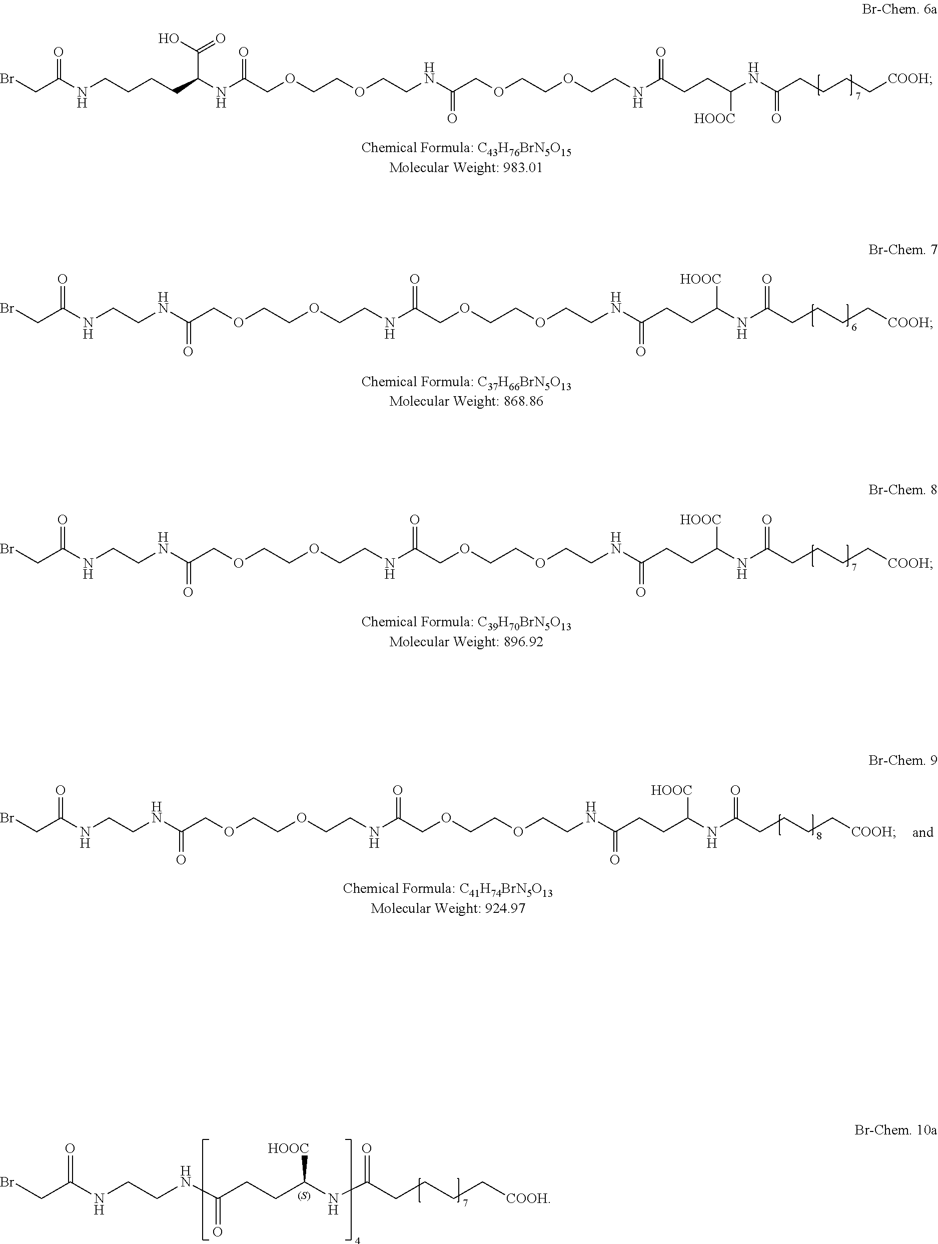

In the above formulas for the albumin binders, the amino acids taking part of the formulas are L-amino acids if the formulas end with “a”.

In one aspect, the albumin binding moiety is:

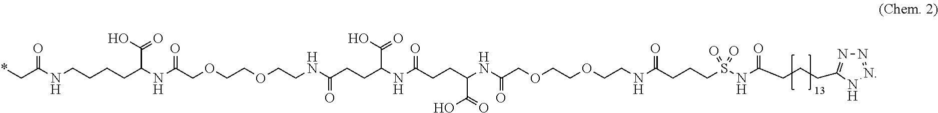

In one embodiment, the long-acting growth hormone receptor antagonist of the present disclosure is provided wherein the albumin binding moiety is defined by a formula selected from the group consisting of:

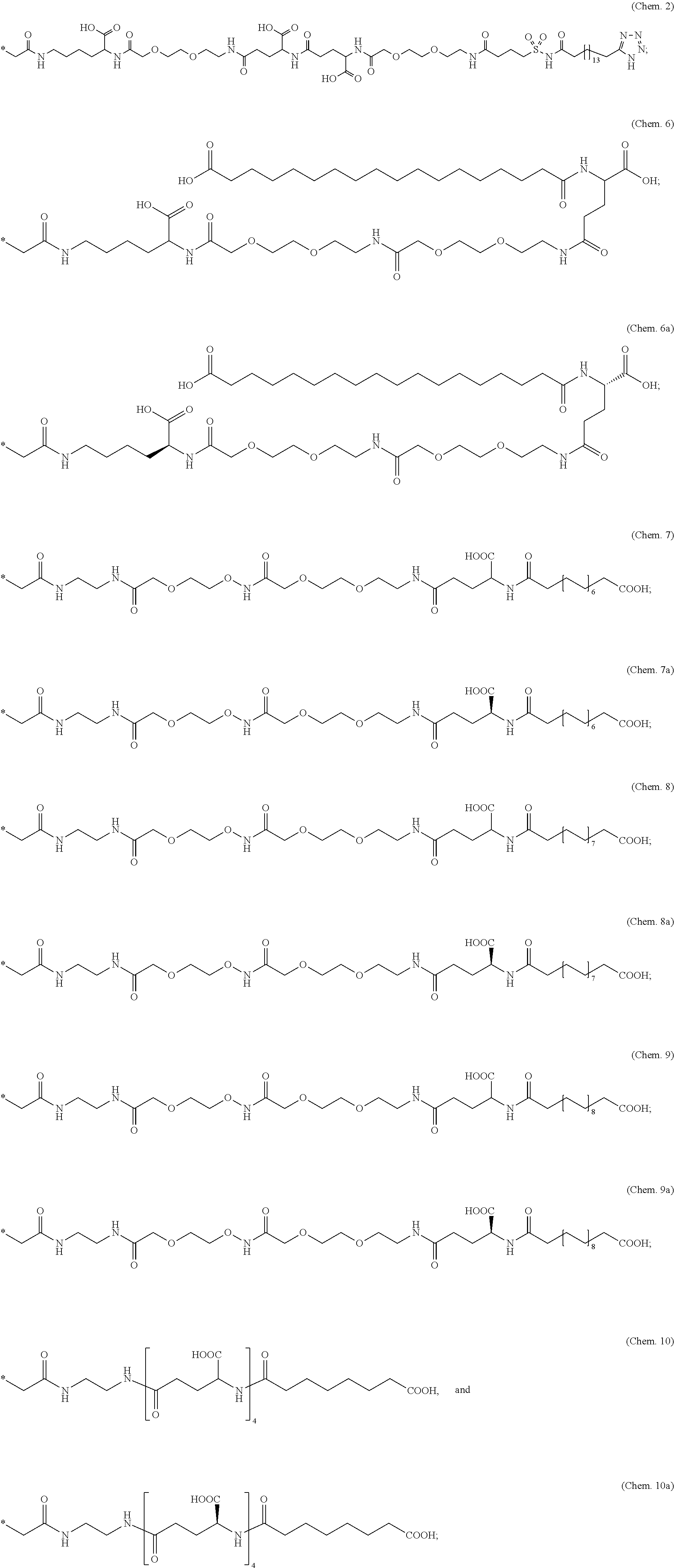

wherein * denotes the attachment point of the albumin binding moiety to the growth hormone variant via the sulphur residue of the cysteine side chain at position 101 of the growth hormone variant.

### Compounds

In one embodiment, the present disclosure provides a long-acting growth hormone receptor antagonist comprising:
a. a growth hormone variant comprising at least the following amino acid substitutions as compared to human growth hormone (hGH) (SEQ ID NO: 1): L101C, G120R/G120K, and wherein the growth hormone variant has at least 80% sequence identity to the polypeptide of SEQ ID NO: 1; and
b. an albumin binding moiety configured for binding to albumin, for example binding to albumin with a dissociation constant (Kd) of less than 1 μM, wherein the albumin binding moiety has a molecular weight of 3 kDa or less and is covalently attached to the growth hormone variant via the sulphur residue of the cysteine side chain at position 101 of the growth hormone variant;
   or a pharmaceutically acceptable salt thereof.

In one embodiment, the growth hormone variant has at least 81% sequence identity to the polypeptide of SEQ ID NO: 1, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity to the polypeptide of SEQ ID NO: 1.

In one embodiment, the LA-GHRA of the present invention is selected from a compound specifically disclosed herein.

In the following, the formulas specifically describe compounds 1-19 where the mutations depicted in brackets refer to specific mutations compared to human growth hormone, i.e. the polypeptide of SEQ ID NO: 1. To exemplify, the notation “-S101 [H18D, H21N, L101C, G120R, R167N, K168A, D171S, K172R, E174S, 1179T]” specifically refers to a polypeptide having the sequence of SEQ ID NO: 1 (human growth hormone) wherein each of the positions in brackets “[ ]” have been mutated as specified by the one-letter amino acid code. The notation further specifies that the albumin binding moiety is bound to cysteine at position 101 as the albumin binding moiety is drawn covalently attached to S101.

In one embodiment, the LA-GHRA is selected from the group consisting of:

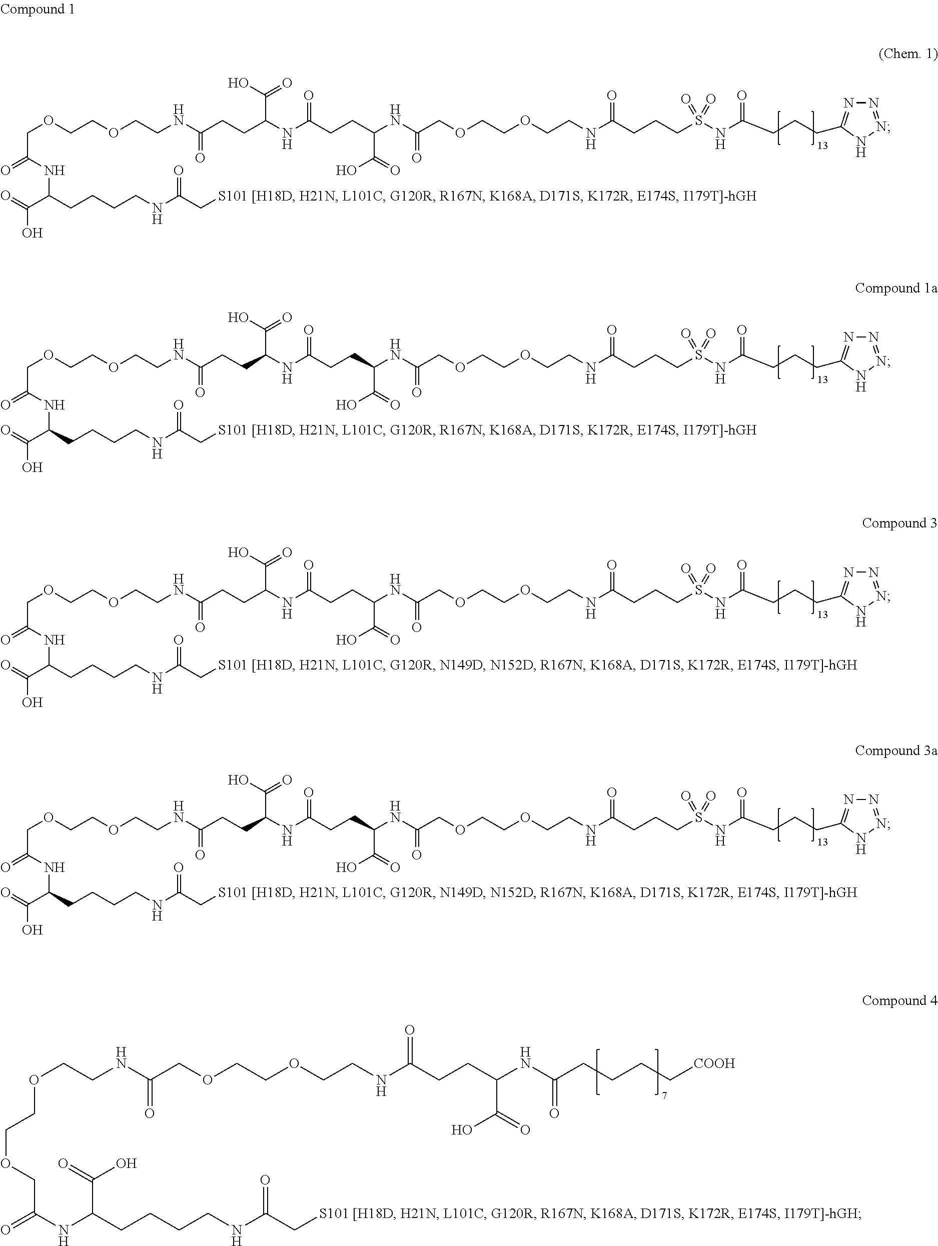

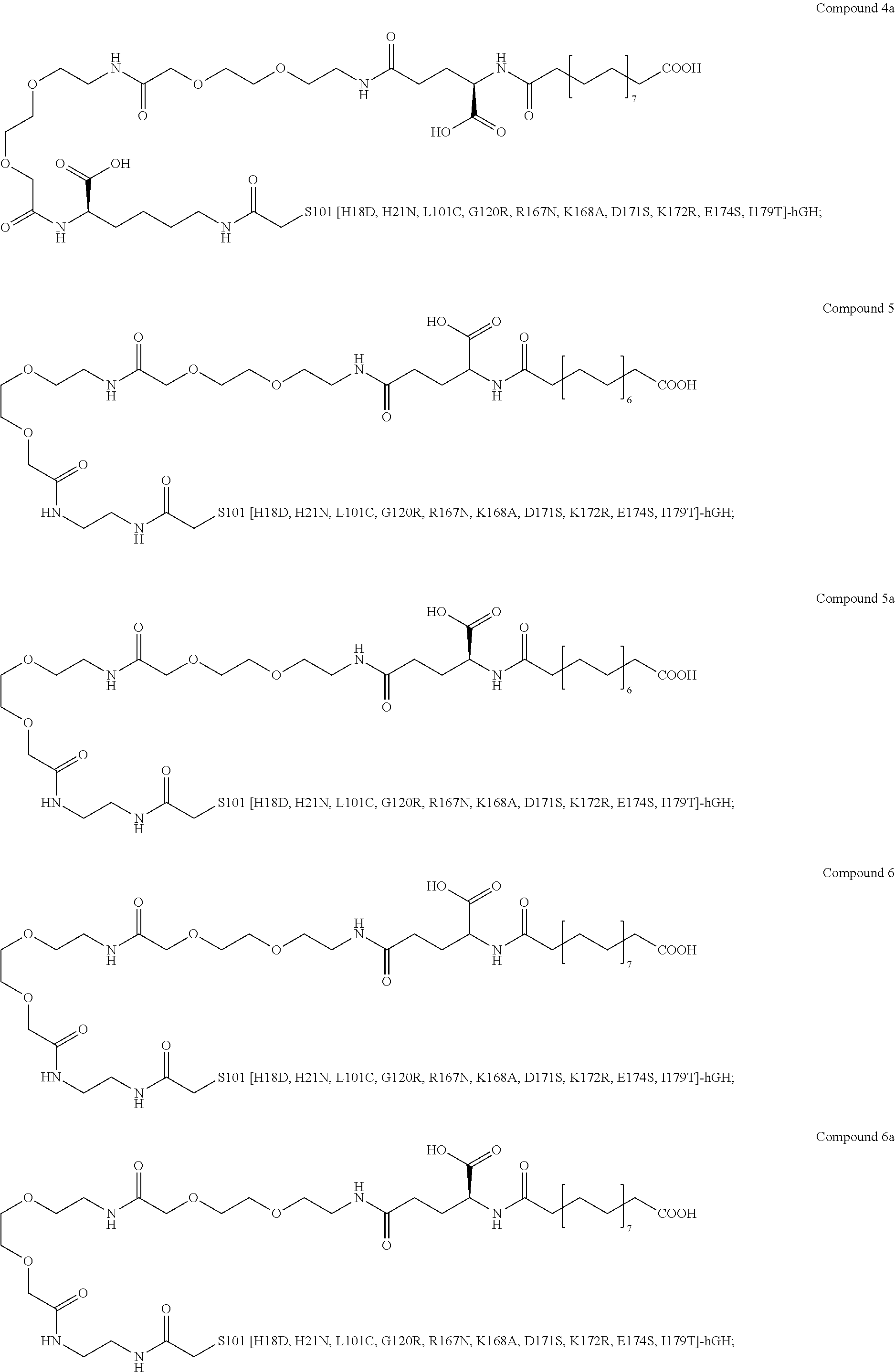

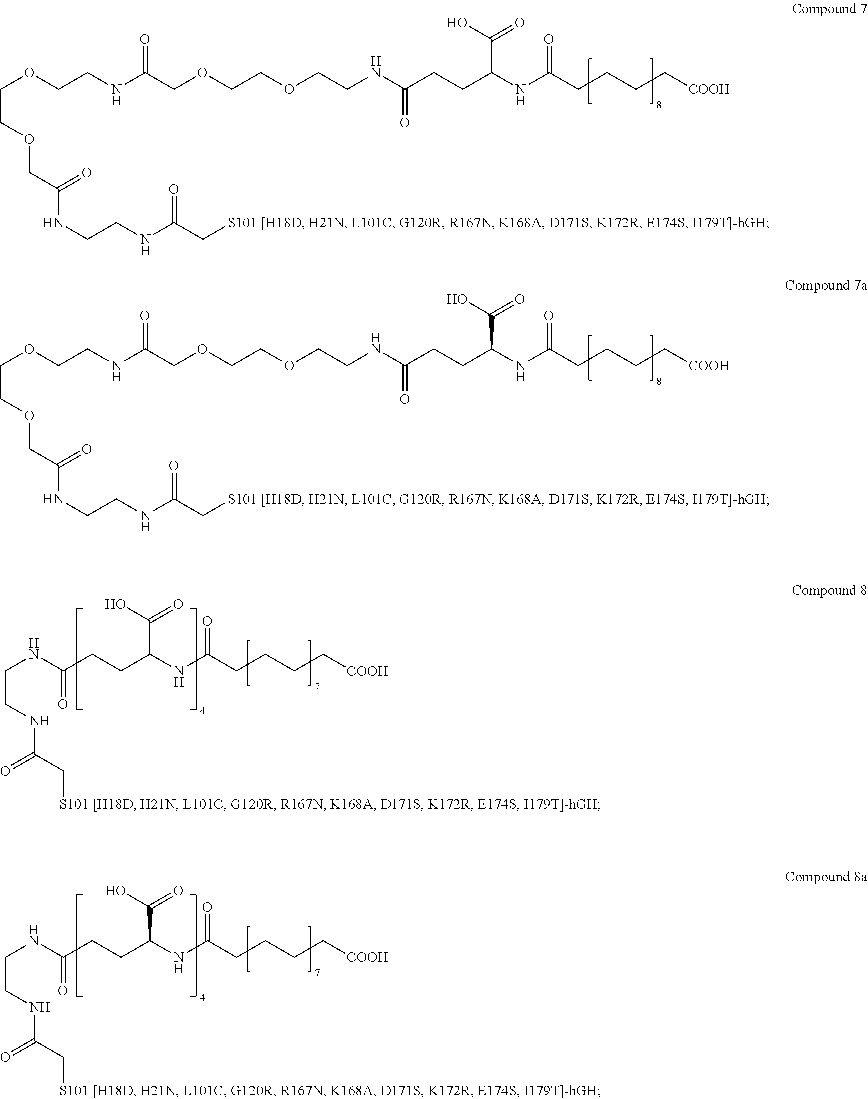

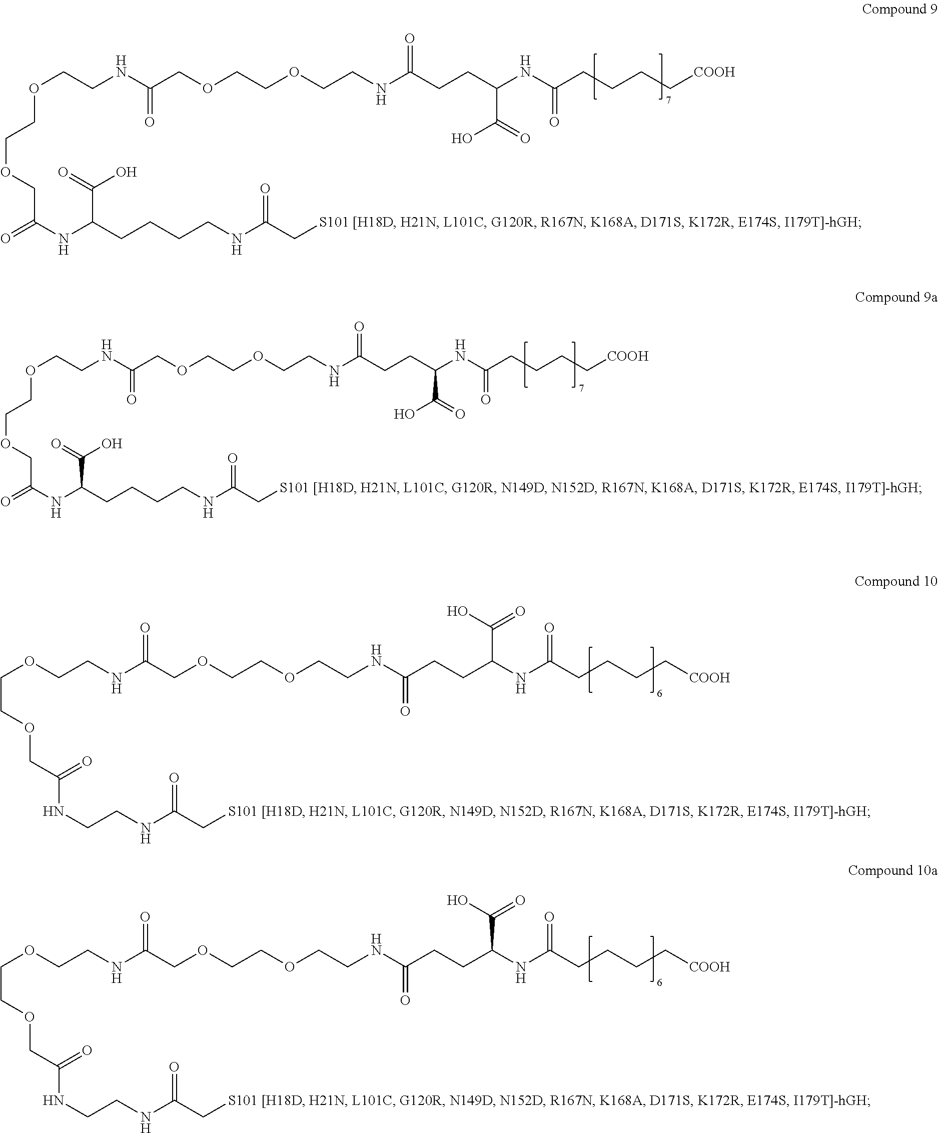

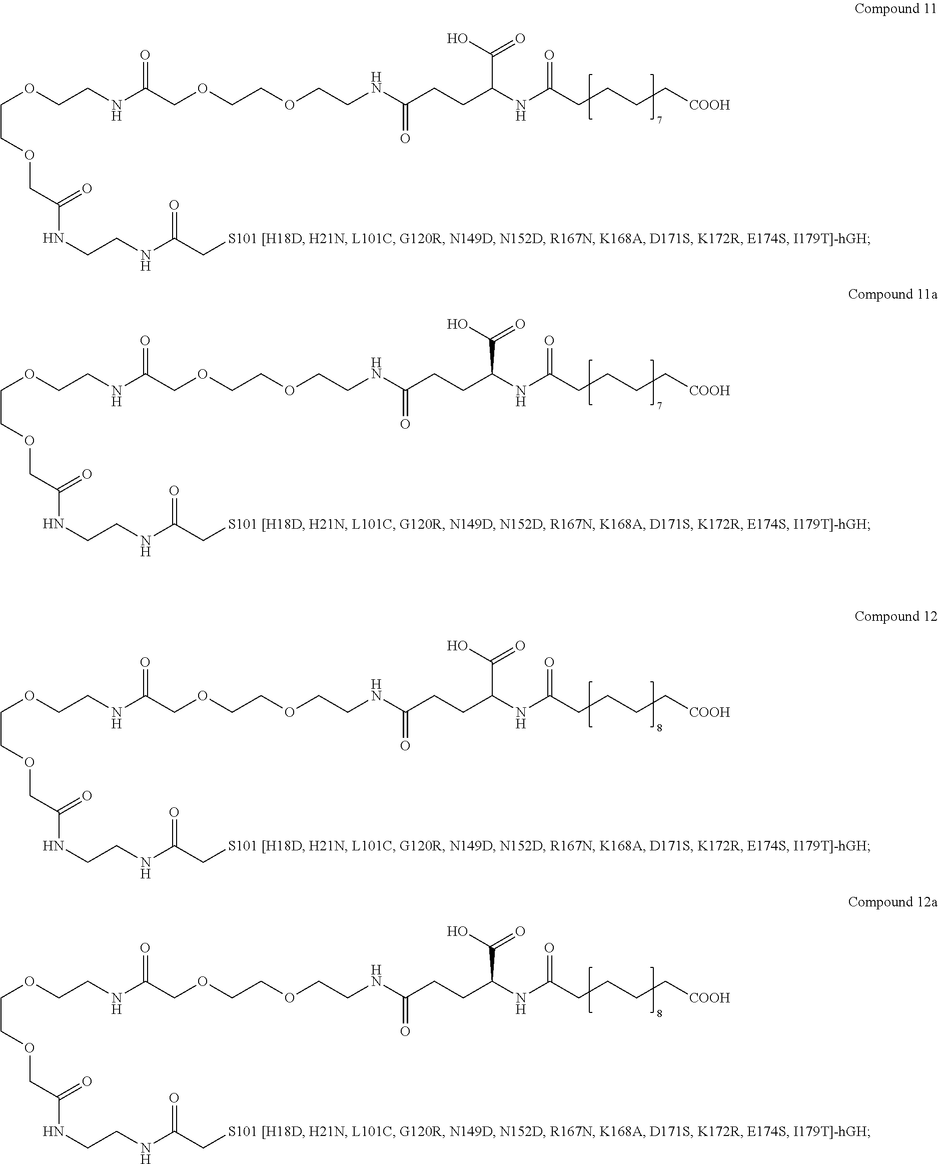

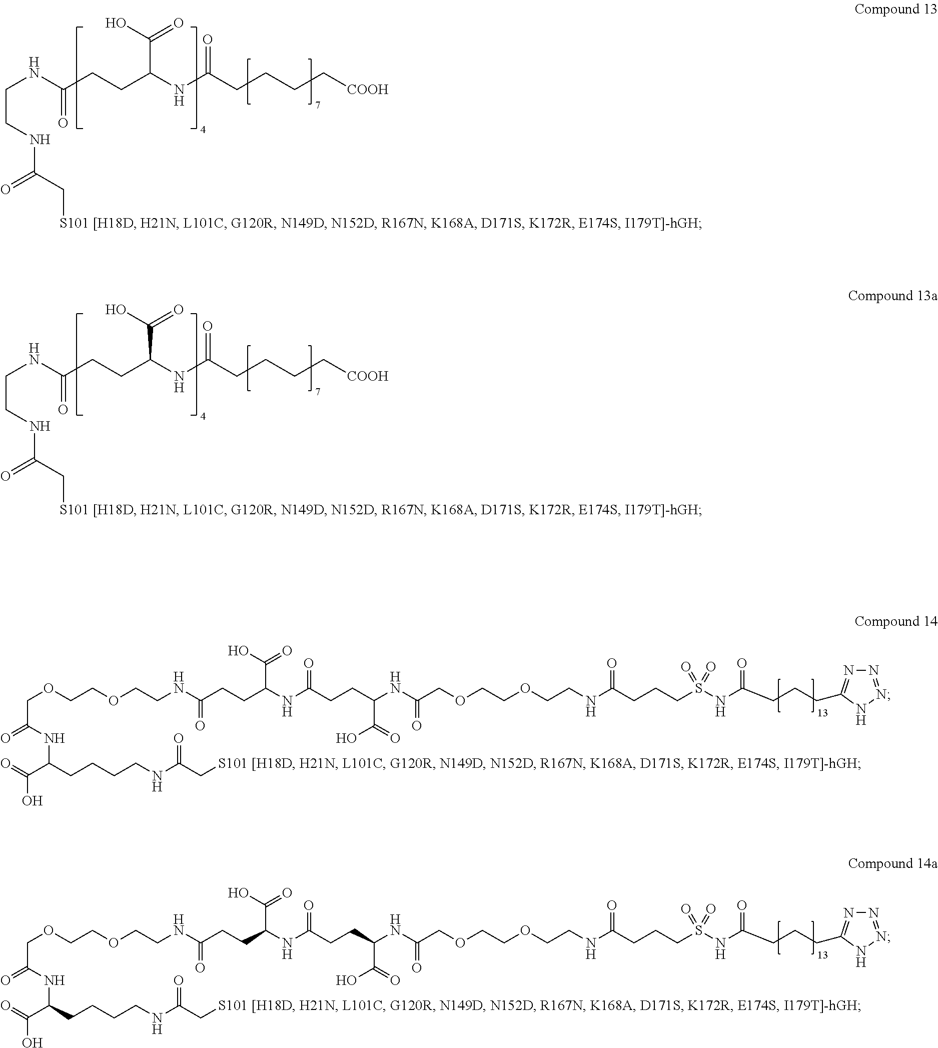

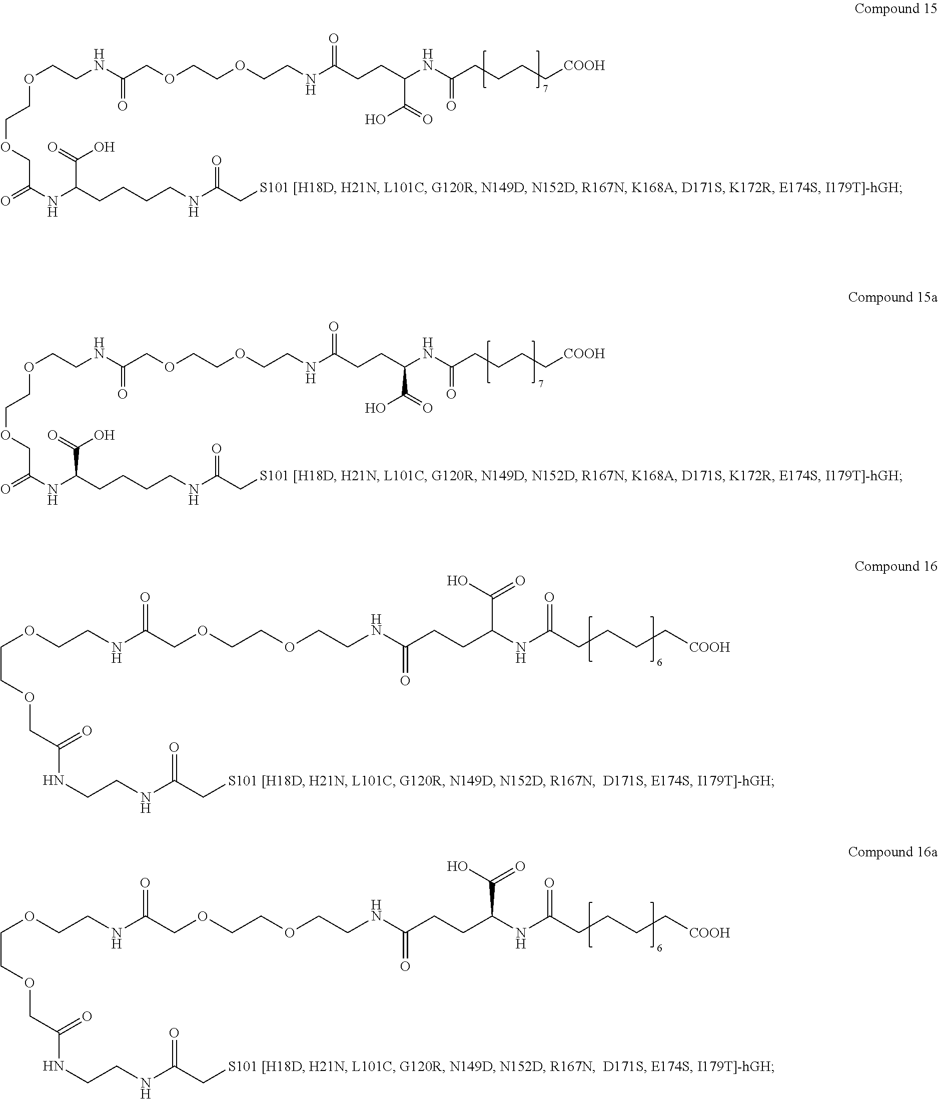

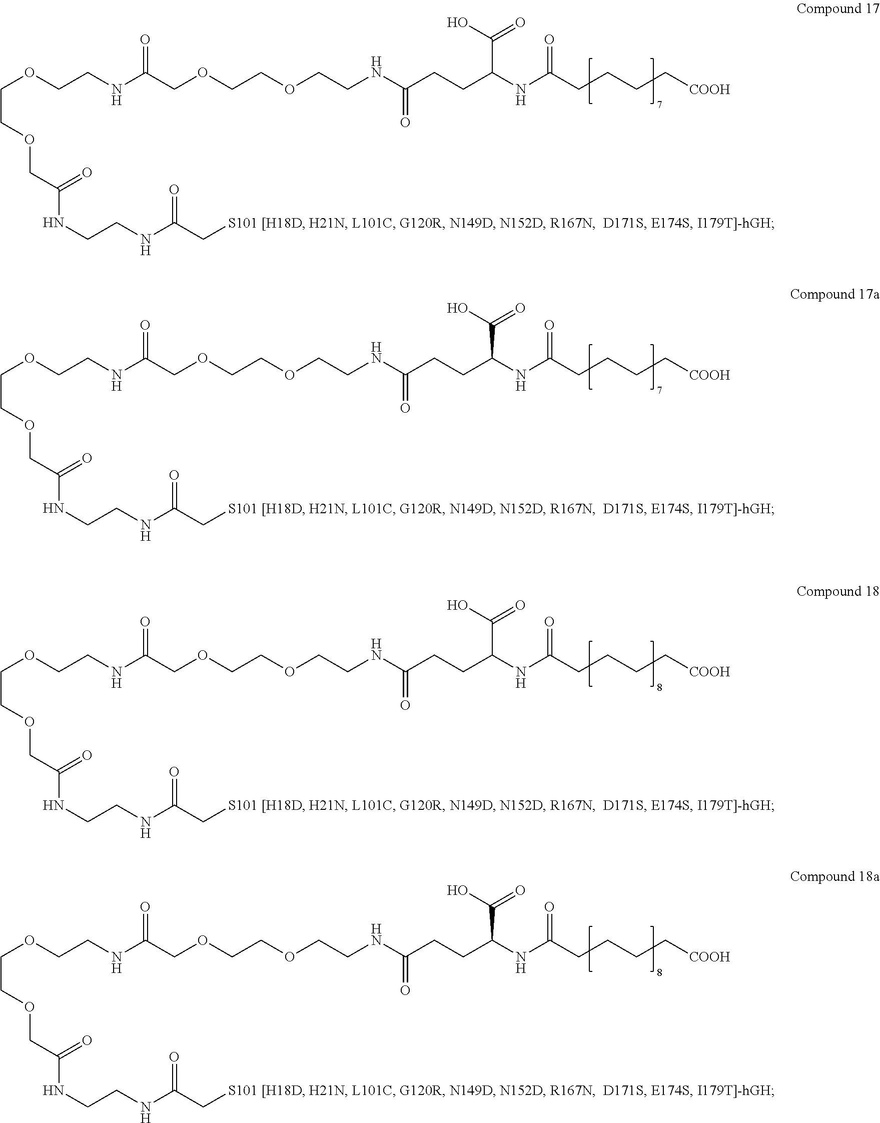

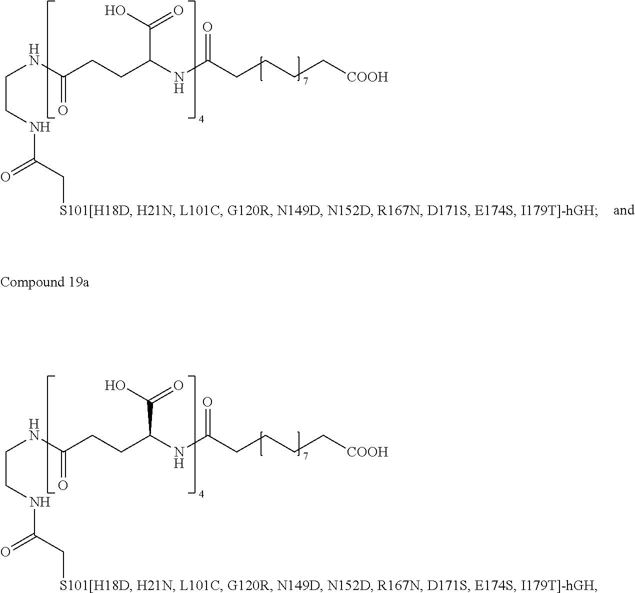

or a pharmaceutically acceptable salt thereof.

In one embodiment, the LA-GHRA of the invention is:

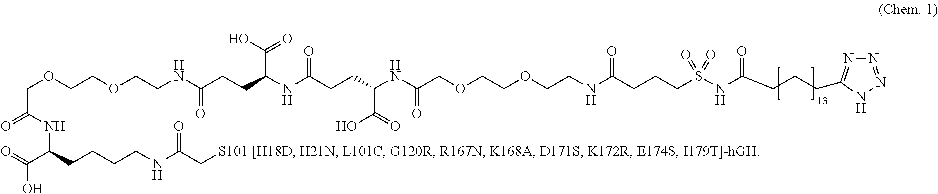

In one embodiment, a long-acting growth hormone receptor antagonist is provided comprising
a. a growth hormone variant comprising the following amino acid substitutions as compared to human growth hormone (hGH) (SEQ ID NO: 1): H18D, H21N, L101C, G120R/G120K, R167N, D171S, E174S, 1179T, and
b. an albumin binding moiety defined by Chem. 2

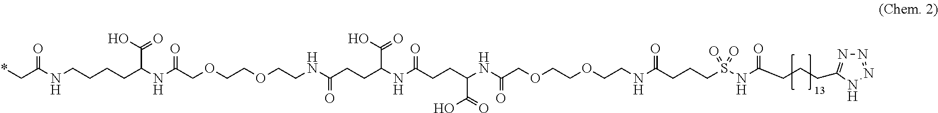

wherein * denotes the attachment point of the albumin binding moiety to the growth hormone variant via the sulphur residue of the cysteine side chain at position 101 of the growth hormone variant; or a pharmaceutically acceptable salt thereof.

In one embodiment, the LA-GHRA is provided wherein the growth hormone variant comprises the following amino acid substitutions as compared to human growth hormone (hGH) (SEQ ID NO: 1): H18D, H21N, L101C, G120R, R167N, K168A, D171S, K172R, E174S, and 1179T.

In one embodiment, the LA-GHRA is compound 1 (Chem. 1a) as described in Example 1 herein also referred to as compound 1a. In one embodiment, the LA-GHRA comprises one or more disulphide bridge between Cys53-Cys165 and/or Cys182-Cys189. In one embodiment, compound 1 comprises one or more disulphide bridges between Cys53-Cys165 and/or Cys182-Cys189.

In one embodiment, the growth hormone variant consists of [H18D, H21N, L101C, G120R, R167N, K168A, D171S, K172R, E174S, 1179T]-hGH (SEQ ID NO: 2), and the albumin binding moiety is Chem. 2.

In one embodiment, the long-acting growth hormone receptor antagonist is Chem. 1:

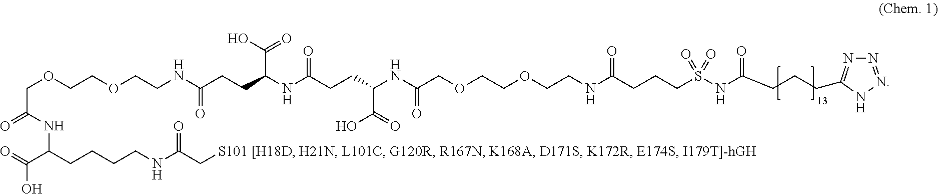

In one embodiment, the long-acting growth hormone receptor antagonist is compound 1 (Chem. 1a):

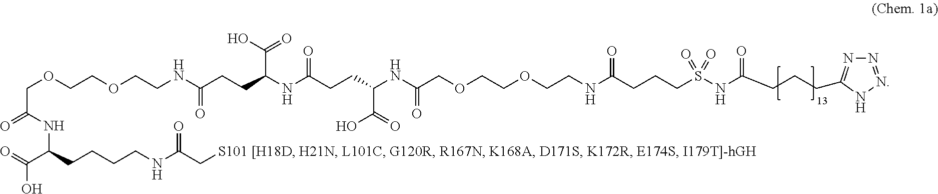

In one embodiment, a long-acting growth hormone receptor antagonist of compound 1a is provided:

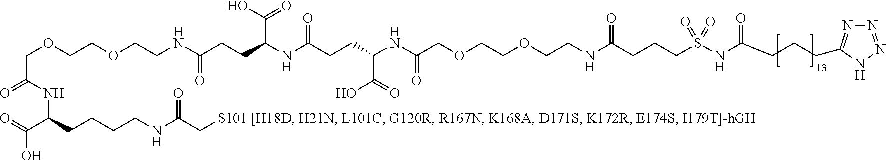

(compound 1a), or a pharmaceutically acceptable salt thereof, optionally having one or two disulphide bridges between Cys53-Cys165 and/or Cys182-Cys189.

In one embodiment, the growth hormone variant comprises one or two disulphide bridges between Cys53-Cys165 and/or Cys182-Cys189. In one embodiment, the growth hormone variant comprises two disulphide bridges between Cys53-Cys165 and Cys182-Cys189. In one embodiment, the growth hormone variant comprises a disulphide bridge between Cys53-Cys165. In one embodiment, the growth hormone variant comprises a disulphide bridge between Cys182-Cys189.

### Functional Properties

In a first functional aspect, the LA-GHRA of the invention, e.g., compound 1a, or the polypeptide backbone thereof show strong binding affinity to the hGHR. This may be determined by various methods known in the art e.g., as described in Examples 2 or 3 herein. The binding affinity may be expressed by the K_{D} value, and it is desired to have a strong binding to the hGHR corresponding to a low K_{D} value.

In a second functional aspect, the LA-GHRA of the invention, e.g., compound 1a, or the polypeptide backbone thereof does not activate the growth hormone receptor or show a reduced activation as compared to the natural response of hGH, i.e., being a GHR antagonist. Particular this may be determined by in vitro activity assays (antagonist mode) such as described in Examples 4 and 5 herein. Generally, the activity or potency (antagonist mode) should be as good as possible, i.e., corresponding to a low IC₅₀ value.

In a third functional aspect, the LA-GHRA of the invention, e.g., compound 1a, or the polypeptide backbone thereof, have desirable pharmacokinetic properties such as increased terminal half-life as compared to hGH. Increased terminal half-life means that the compound in question is eliminated slower from the body and thus extend duration of the pharmacological effect for the compound. A desirable half-life means a rate that makes it suitable e.g., for daily or weekly administration, preferably weekly administration. In a particular embodiment, the pharmacokinetic properties may be determined as terminal half-life (t_{1/2}) in vivo in Sprague-Dawley rats after i.v. or s.c. administration, e.g., as described in Example 5 herein. In one embodiment, the half-life in Sprague-Dawley rats is at least 5h, such as at least 8 h or at least 12 h.

In one embodiment, the growth hormone receptor antagonist is binding to the hGH receptor in an SPR assay, for example as described in Example 2 herein.

In one embodiment, the growth hormone receptor antagonist is binding to the hGH receptor in a binding affinity ITC assay, such as described in Example 3 herein.

In one embodiment, the growth hormone receptor antagonist is an antagonist at the growth hormone receptor.

In one embodiment, the growth hormone receptor antagonist is capable of inhibiting hGH activity.

In one embodiment, the growth hormone receptor antagonist is capable of inhibiting hGH activity in a STAT3 activity assay, such as described in Example 4 herein.

In one embodiment, the growth hormone receptor antagonist is capable of inhibiting hGH activity in a BAF-3 GHR activity assay, such as described in Example 5 herein.

In one embodiment, the growth hormone receptor antagonist has improved pharmacokinetic properties.

In one embodiment, the growth hormone receptor antagonist has an increased terminal half-life when determined in rats, such as described in Example 6 herein.

### Production Processes

The LA-GHRA of the invention may be produced by a combination of recombinant techniques followed by chemical modification, e.g., as described in example 1 herein. The growth hormone variant, such as SEQ ID NO: 2, may be prepared by recombinant methods, viz. by culturing a host cell containing a DNA sequence encoding the growth hormone variant, and capable of expressing the polypeptide in a suitable nutrient medium under conditions permitting the expression of the polypeptide. Non-limiting examples of host cells suitable for expression of these polypeptides are: Escherichia coli, Saccharomyces cerevisiae, as well as mammalian BHK or CHO cell lines. Specific example of a method of preparing SEQ ID NO: 2 is included in the experimental part. Specific examples of a method of preparing SEQ ID NO: 3 and 4 are also included in the experimental part.

In one embodiment, a process for preparing a human growth hormone variant selected from the group consisting of SEQ ID NO: 2, SEQ ID NO: 3, and SEQ ID NO: 4 is provided comprising culturing a host cell comprising a vector as defined herein.

In one embodiment, a process is provided for preparing a human growth hormone variant of SEQ ID NO: 2 comprising culturing a host cell comprising a vector as specifically defined herein.

### Pharmaceutical Compositions

Injectable pharmaceutical compositions comprising the antagonist of the present invention can be prepared using the conventional techniques of the pharmaceutical industry which involves dissolving and mixing the ingredients as appropriate to give the desired end product. Thus, according to one procedure, the antagonist of the present invention is dissolved in a suitable buffer at a suitable pH, so precipitation is minimized or avoided. The injectable composition is made sterile, for example, by sterile filtration.

Pharmaceutical compositions comprising the antagonist of the invention or a pharmaceutically acceptable salt, and a pharmaceutically acceptable excipient may be prepared as is known in the art.

The term “excipient” broadly refers to any component other than the active therapeutic ingredient(s). The excipient may be an inert substance, an inactive substance, and/or a not medicinally active substance.

The formulation of pharmaceutically active ingredients with various excipients is known in the art, see e.g., Remington: The Science and Practice of Pharmacy (e.g., 19th edition (1995), and any later editions).

A composition may be a stabilised formulation. The term “stabilised formulation” refers to a formulation with increased physical and/or chemical stability, preferably both. In general, a formulation must be stable during use and storage (in compliance with recommended use and storage conditions) until the expiration date is reached.

The pharmaceutical composition may be administered to an individual via parenteral administration including subcutaneous, intramuscular, intraperitoneal, or intravenous injection by means of a syringe, optionally a pen-like syringe. Alternatively, parenteral administration can be performed by means of an infusion pump.

In one aspect of the invention, the antagonist of the invention is combined with a somatostatin analogue e.g., in the form of a kit-of-parts comprising a preparation of the antagonist of the invention as a first unit dosage form and a preparation of the somatostatin analogue as a second unit dosage form.

Non-limiting examples of somatostatin analogues to be combined with the antagonist of the present invention are octreotide, lanreotide, pasireotide or derivatives thereof.

In one embodiment, a pharmaceutical composition is provided comprising the long-acting growth hormone receptor antagonist as defined herein and a pharmaceutically acceptable excipient. In particular embodiments, the long-acting growth hormone receptor antagonist is compound 1a.

### Pharmaceutical Indications

The present invention also relates to a long-acting growth hormone receptor antagonist for use as a medicament. In one embodiment, the present disclosure provides a long-acting growth hormone receptor antagonist as specifically defined herein, such as compound 1a, for use as a medicament.

Also, or alternatively, the present invention relates to a long-acting growth hormone variant being an antagonist at the growth hormone receptor for use as a medicament. In a particular embodiment, the present invention relates to compound 1a for use as a medicament. In particular, the antagonist of the invention may be used for the treatment of diseases caused by excess levels of growth hormone and/or IGF-1.

In particular embodiments, the long-acting growth hormone receptor antagonist of the invention may be used for treatment of the following medical indications such as but not limited to acromegaly, gigantism, various cancer types where IGF-1 is a growth factor, diabetes mellitus, diabetic retinopathy, diabetic nephropathy.

In a particular embodiment, the indication is acromegaly.

### Nucleic Acids, Vectors, and Host Cells

In one embodiment, a nucleic acid is provided encoding a human growth hormone variant comprising the following amino acids modifications compared to hGH:
H18D, H21N, L101C, G120R, R167N, K168A, D171S, K172R, E174S, and I179T (GHv-1);
H18D, H21N, L101C, G120R, N149D, N152D, R167N, K168A, D171S, K172R, E174S, and 1179T (GHv-2); or
H18D, H21N, L101C, G120R, N149D, N152D, R167N, D171S, E174S, and |179T (GHv-3).

In one embodiment, a nucleic acid is provided encoding a human growth hormone variant comprising the following amino acids modifications compared to hGH:
H18D, H21N, L101C, G120R, R167N, K168A, D171S, K172R, E174S, and 1179T.

In one embodiment, a vector is provided comprising a nucleic acid encoding a human growth hormone variant comprising the following amino acid modification compared to hGH:
H18D, H21N, L101C, G120R, R167N, K168A, D171S, K172R, E174S, and 1179T (GHv-1);
H18D, H21N, L101C, G120R, N149D, N152D, R167N, K168A, D171S, K172R, E174S, and 1179T (GHv-2); or
H18D, H21N, L101C, G120R, N149D, N152D, R167N, D171S, E174S, and 1179T (GHv-3).

In one embodiment, a vector is provided comprising a nucleic acid encoding a human growth hormone variant comprising the following amino acid modification compared to hGH:
H18D, H21N, L101C, G120R, R167N, K168A, D171S, K172R, E174S, and 1179T.

In one embodiment, the vector is provided wherein the human growth hormone variant is selected from the group consisting of: SEQ ID NO: 2, SEQ ID NO: 3, and SEQ ID NO: 4. In one embodiment, the vector is provided wherein the human growth hormone variant is SEQ ID NO: 2.

In one embodiment, a host cell is provided comprising a vector as defined herein.

### EMBODIMENTS

The invention is further described by the following non-limiting embodiments:
1. A long-acting growth hormone receptor antagonist comprising
   a. a growth hormone variant comprising the following amino acid substitutions as compared to human growth hormone (hGH) (SEQ ID NO: 1): L101C, G120R/G120K, and
   b. an albumin binding moiety defined by Chem. 2

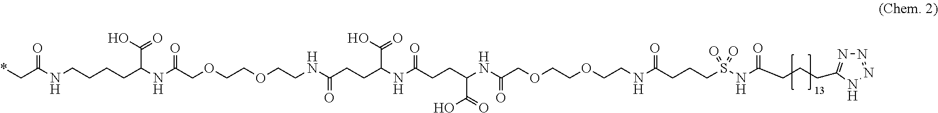

wherein * denotes the attachment point of the albumin binding moiety to the growth hormone variant via the sulphur residue of the cysteine side chain at position 101 of the growth hormone variant;
   or a pharmaceutically acceptable salt thereof.
2. The antagonist according to embodiment 1 comprising
   a. a growth hormone variant comprising the following amino acid substitutions as compared to human growth hormone (hGH) (SEQ ID NO: 1): H18D, H21N, L101C, G120R/G120K, R167N, D171S, E174S, 1179T.
3. The antagonist according to embodiments 1-2, wherein the growth hormone variant comprises the following amino acid substitutions as compared to hGH: H18D, H21N, L101C, G120R/G120K, R167N, K168A, D171S, K172R, E174S, 1179T.
4. The antagonist according to any of the preceding embodiments, wherein the substitution at position 120 is G120R.
5. The antagonist according to any of the preceding embodiments, wherein the growth hormone variant comprises 1-10, such as 2-8 or 3-5 further amino acid substitutions.
6. The antagonist according to any of the preceding embodiments, wherein the growth hormone variant consists of [H18D, H21N, L101C, G120R, R167N, K168A, D171S, K172R, E174S, 1179T]-hGH (SEQ ID NO: 2), and the albumin binding moiety is Chem. 2.
7. The antagonist according to any one of the preceding embodiments which is Chem. 1:

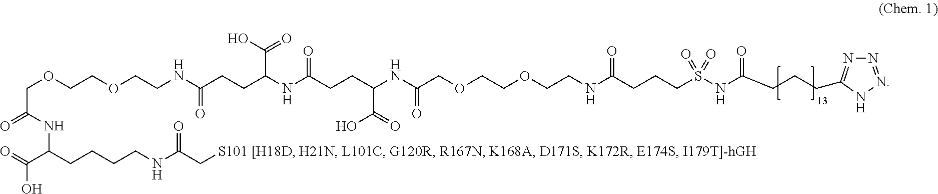

8. The antagonist according to any one of the preceding embodiments which is compound 1 (Chem. 1a).
9. The antagonist according to any one of the preceding embodiments, wherein the growth hormone variant comprises one or two disulphide bridges between Cys53-Cys165 and/or Cys182-Cys189.
10. The antagonist according to any one of the preceding embodiments, wherein the growth hormone variant comprises two disulphide bridges between Cys53-Cys165 and Cys182-Cys189.
11. The antagonist according to any of embodiments 1-9, wherein the growth hormone variant comprises a disulphide bridge between Cys53-Cys165.
12. The antagonist according to any of embodiments 1-9, wherein the growth hormone variant comprises a disulphide bridge between Cys182-Cys189.
13. The antagonist according to any of the preceding embodiments, wherein the growth hormone variant is 191 amino acids long.
14. The antagonist according to any one of the preceding embodiments, wherein the growth hormone variant is a growth hormone receptor antagonist.
15. The antagonist according to any of the preceding embodiments which is capable of binding to the growth hormone receptor.
16. The antagonist according to any of the preceding embodiments which is binding to the hGH in an SPR assay as described in Example 2 herein.
17. The antagonist according to any of the preceding embodiments which is binding to the hGH in a binding affinity ITC assay such as described in Example 3 herein.
18. The antagonist according to any of the preceding embodiments which is an antagonist at the growth hormone receptor.
19. The antagonist according to any of the preceding embodiments which is capable of inhibiting hGH activity.
20. The antagonist according to any of the preceding embodiments which is capable of inhibiting hGH activity in a STAT3 activity assay such as described in Example 4 herein.
21. The antagonist according to any of the preceding embodiments which is capable of inhibiting hGH activity in a BAF-3 GHR activity assay such as described in Example 5 herein.
22. The antagonist according to any of the preceding embodiments which has improved pharmacokinetic properties.
23. The antagonist according to any of the preceding embodiments which has an increased terminal half-life when determined in rats such as described in Example 6 herein.
24. A pharmaceutical composition comprising a long-acting growth hormone receptor antagonist according to any one of embodiments 1-23, and a pharmaceutically acceptable excipient.
25. A compound according to any one of embodiments 1-23 for use as a medicament.
26. A compound according to any one of embodiments 1-23 for use in the treatment or prevention of diseases caused by excess human growth hormone and/or IGF-1 levels, such as acromegaly or gigantism.
27. The compound for use according to embodiment 26 in combination with a somatostatin analogue, such as octreotide, lanreotide, pasireotide or derivatives thereof.
28. The compound for use according to embodiment 27, wherein the somatostatin analogue is octreotide.
29. Use of the compound according to any one of embodiments 1-23 for the preparation of a medicament.
30. Use of the compound according to any one of embodiments 1-23 for the preparation of a medicament for the treatment or prevention of diseases caused by excess human growth hormone and/or IGF-1, such as acromegaly or gigantism.
31. A method of treatment of diseases caused by excess human growth hormone and/or IGF-1 levels, such as acromegaly or gigantism, comprising administering a pharmaceutically active amount of a compound according to any of embodiments 1-23.
32. A method of treatment of diseases caused by excess human growth hormone and/or IGF-1 levels, such as acromegaly or gigantism, comprising a pharmaceutically active amount of compound 1 (Chem. 1a).
33. A nucleic acid encoding a human growth hormone variant comprising the following amino acids modifications compared to hGH: H18D, H21N, L101C, G120R, R167N, K168A, D171S, K172R, E174S, 1179T.
34. The nucleic acid according to embodiment 33 wherein the growth hormone variant is SEQ ID NO: 2.
35. A vector comprising a nucleic acid encoding a human growth hormone variant comprising the following amino acid modification compared to hGH: H18D, H21N, L101C, G120R, R167N, K168A, D171S, K172R, E174S, 1179T.
36. The vector according to embodiment 35 wherein the human growth hormone variant is SEQ ID NO: 2.
37. A host cell comprising a vector according to any one of embodiments 35-36.
38. A process for preparing a human growth hormone variant of SEQ ID NO: 2 comprising culturing a host cell comprising a vector according to any one of embodiments 35-36.
39. The process according to embodiment 38 further comprising alkylation of the variant according to SEQ ID NO: 2 at Cys101 with an albumin binding moiety according to Chem. 2, such as Chem. 2a, e.g. using a haloacetamide according to e.g. Chem. 3, Chem. 3a, Chem. 4, Chem 4a, Chem. 5 or Chem. 5a.

### EXAMPLES

List of Abbreviations:

| | |
|---|---|
| Ado: | (2[2-(amino)ethoxy]ethoxy)acetyl |
| CAD: | Charged Aerosol Detector |
| DCM: | dichloromethane, CH₂Cl₂, methylene chloride |
| DIC: | diisopropylcarbdiimide |
| DIPEA: | N,N-diisopropylethylamine |
| DMF: | N,N-dimethylformamide |
| DTT: | 1,4-dithiothreitol |
| ECD: | Extracellular domain |
| EDTA: | ethylenediaminetetraacetic acid |
| eq.: | Equivalent(s) |
| Et₂O: | diethyl ether |
| FBS: | Fetal Bovine Serum |
| Fmoc: | 9H-fluoren-9-ylmethoxycarbonyl |
| gGlu: | gamma-Glutamic acid; γGlu |
| GH: | Growth Hormone |
| h: | hour(s) |
| hGH: | human Growth Hormone |
| hGHR: | human Growth Hormone Receptor |
| hGHRA: | human Growth Hormone Receptor Antagonist |
| HPLC: | high pressure liquid chromatography |
| IGF-1: | Insulin-like Growth Factor 1 |
| IPTG: | Isopropyl-β-D-1-thiogalactopyranoside |
| i.v.: | intravenous |
| LBAmp: | Lysogeny Broth medium containing Ampicillin |
| LCMS: | liquid chromatography - mass spectrometry |
| m/z: | mass to charge ratio |
| MALS: | Multi-Angle Light Scattering |
| MeCN: | acetonitrile |
| min: | minute(s) |
| MTT: | 4-Methyltrityl |
| NHS: | N-hydroxysuccinimide |
| NMP: | N-methylpyrrolidin-2-one |
| OD: | Optical Density |
| OtBu: | tert butyl ester |
| PBS: | Phosphate-buffered saline |
| Rcf: | relative centrifugal force |
| rpm: | rounds per minute |
| rt: | room temperature |
| SEC: | Size Exclusion Chromatography |
| s.c.: | subcutaneous |
| Su: | N-Hydroxysuccinimide |
| TBAmp: | Terrific Broth medium containing Ampicillin |
| tBu: | tert butyl |
| TFA: | trifluoroacetic acid |
| TIS: | triisopropylsilane |
| TNBS: | trinitrobenzensulfonic acid |
| UPLC: | Ultra-performance liquid chromatography |

### General Methods:

### LCMS Method:

Characterisation was done by LCMS analysis using a Waters Acquity UPLC H Class, with a Waters Aquity UPLC Protein BEH column (C4, 300 Å, 1.7 μm, 2.1 mm×50 mm) equipped with a Waters Xevo G2-XS QTof detector. A water/MeCN system (linear gradient from 5-95% MeCN over 6 min containing 0.1% TFA) was used as eluent. The column was operated at a flow rate of 0.4 ml/min and at a temperature of 60° C. Mass spectra were recorded in the 200-3000 Da range and deconvoluted using MassLynx version 4.2.

### UPLC Method:

Purity was addressed by UPLC analysis using a Waters UPLC system equipped with a Acquity UPLC BEH300 C4 1.7 μm, 2.1×50 mm column. The system was further equipped with a Waters Acquity PDA Detector setup to detect at 214 nm/280 nm. The eluents consisted of 0.05% TFA in Milli-Q Water (A) and 0.05% TFA in acetonitrile (B). A linear gradient system running from 25% to 75% B, with a gradient run-time of 6 min. was employed. The column was operated at a flow rate of 0.4 ml/min and at a temperature of 40° C. Purity was defined as peak AUC in relation to total AUC exclusive solvent peak (in percent) as reported by system software for each UV wavelength.

### CAD Method:

Concentrations were determined using a CAD detection system. The samples were run on a Vanquish Thermo-Fisher system, using a Waters CSH C4, 50×2.1 mm, 1.7 μm column equipped with a CAD/Corona detector. A water/MeCN system (linear gradient from 5-95% MeCN over 6 min containing 0.1% TFA) was used as eluent. The column was operated at a flow rate of 0.45 ml/min and at a temperature of 40° C. Quantifications were done relative to Insulin Aspart standard.

### Example 1: Preparation of GH Receptor Antagonist Compound 1 (Chem. 1a)

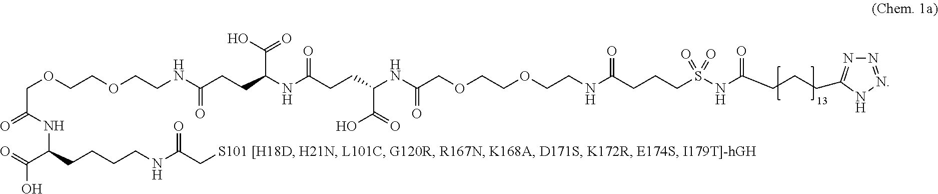

Compound 1a was made using the following general steps:
i) GHRA peptide backbone expression in E. coli to obtain the Cys101 cystamine protected mixed disulphide.
ii) Synthesis of the iodoacetamide of the albumin binding moiety (Chem. 5a).
iii) Deprotection of the Cys101 mixed disulphide and nucleophilic substitution with iodoacetamide (Chem. 5a).

Details of each step are shown below or alternatively, the compound can be obtained by using the procedures as described in WO2011/089255, (see page 132, line 17-page 137; Example 44, page 161, line 5-page 164, line 11).

### i) Method for Preparing GH Receptor Antagonist Backbone (Compound 2; SEQ ID NO: 2)

A plasmid vector encoding Hisx6-SUMO-[H18D, H21N, L101C, G120R, R167N, K168A, D171S, K172R, E174S, 1179T]-hGH was prepared and transfected into E. coli. The cells were then cultivated in LBAmp medium at 30° C. overnight, before being transferred into TBAmp medium and cultivated at 37° C. until the optical density (OD) reached 1.8. Expression of the target protein was induced with a final concentration of 0.1 mM IPTG at 16° C. overnight. Cell pellet was then harvested by centrifugation and disrupted by high pressure cell homogenizer. Material was resuspended in 20 mM Tris, 300 mM NaCl, 1 mM cysteamine-2.HCl, pH 8.0 buffer containing 200 mM Arg at a concentration of 1:10 (w/v). The supernatant was collected by centrifugation and subsequently subjected to chromatographic purification on a Ni Excel His tag capture column. The column was washed with 5 bed volume of 20 mM Tris, pH 8.0, 300 mM NaCl, 1 mM cysteamine-2.HCl, 200 mM Arg. The His-SUMO tag was removed using SUMO protease, 1:1000 (w/w) dilution of the protease, 18° C., overnight. To the digesting solution was added NaCl to a concentration of 1M and loaded onto a Phenyl FF column. The column was washed with 2 bed volumes of 20 mM Tris, pH 7.5, 1M NaCl, 200 mM Arg, 1 mM cystamine-2.HCl, and then 2.5 bed volumes of 20 mM Tris, pH 7.5, 1 mM cystamine-2.HCl to elute the protein. The crude protein was further purified by passing it through a SEC Hiload 26/60, Superdex 75 column, eluted with PBS buffer containing 1 mM cysteamine-2.HCl. The target protein [H18D, H21N, L101C, G120R, R167N, K168A, D171S, K172R, E174S, 1179T]-hGH (compound 2) was obtained in the Cys101 cystamine protected (mixed disulphide) form at a concentration of 5.3-6.0 mg/ml as determined by CAD-C4 concentration analysis. The intact purified protein was analysed using LCMS. The observed mass corresponded to the theoretical mass deduced from the amino acid sequence. The expected linkage of disulphide bonds was demonstrated by peptide mapping using trypsin and AspN digestion followed by LCMS analysis of the digest before and after reduction of the disulphide bonds with DTT. LCMS (general method): 22092 Da.

ii) Preparation of Albumin Binder 4-(1H-Tetrazol-16-Yl-Hexadecanoylsulfamoyl) Butanoyl-Ado-γGlu-γGlu-Ado-N^{ε}(C(O)CH₂I) Lys-OH (Chem. 5a)

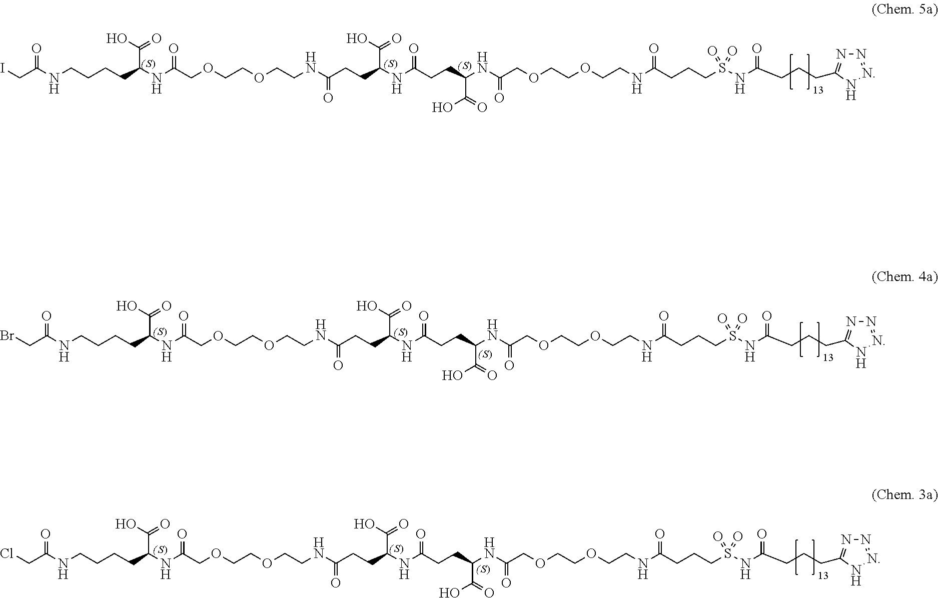

First, the corresponding bromoacetamide (Chem. 4a) was synthesised on solid support according to scheme 1, in 1 mM scale using standard Fmoc-peptide chemistry on an ABI433 synthesizer. Peptide was assembled on a Fmoc-Lys (MTT)-Wang resin using Fmoc-Ado-OH and Fmoc-Glu-OtBu protected amino acids. 4-(16-1H-Tetrazol-5-yl-hexadecanoylsulfamoyl) butyric acid was manually coupled using DIC/NHS in DCM/NMP, 2 eq. overnight, The resin was then treated with 50 mL DCM/TFA/TIS/water (94:2:2:2) in a flowthrough arrangement until the yellow colour disappeared, followed by washing and neutralizing with DIPEA/DMF. The resin was treated with a solution of bromo acetic acid (4 mM) in DCM/NMP (1:1), which was activated with a mixture of NHS and DIC (1 mM of both). The resulting mixture was filtered and then combined with an additional 1 mM of DIPEA. After 1 h, the reaction was completed. The resin was treated with 80 mL TFA/TIS/water (95:2,5:2,5) for 1 h. Evaporated with a stream of N₂, precipitated by addition of Et₂O and washed with Et₂O and dried. Crude product was purified on preparative HPLC (2 runs), with a gradient from 30-80% 0.1 TFA/MeCN against 0.1% TFA in water. Fractions were collected and lyophilized with ~50% MeCN affording Chem. 4a. TOF-MS: mass 1272.52 (M+1).

4-(1H-Tetrazol-16-yl-hexadecanoylsulfamoyl) butanoyl-Ado-γGlu-γGlu-Ado-N^{ε}(C(O)CH₂Cl) Lys-OH (Chem. 3a) can be prepared by same method using chloroacetic acid instead of bromoacetic acid in last step.

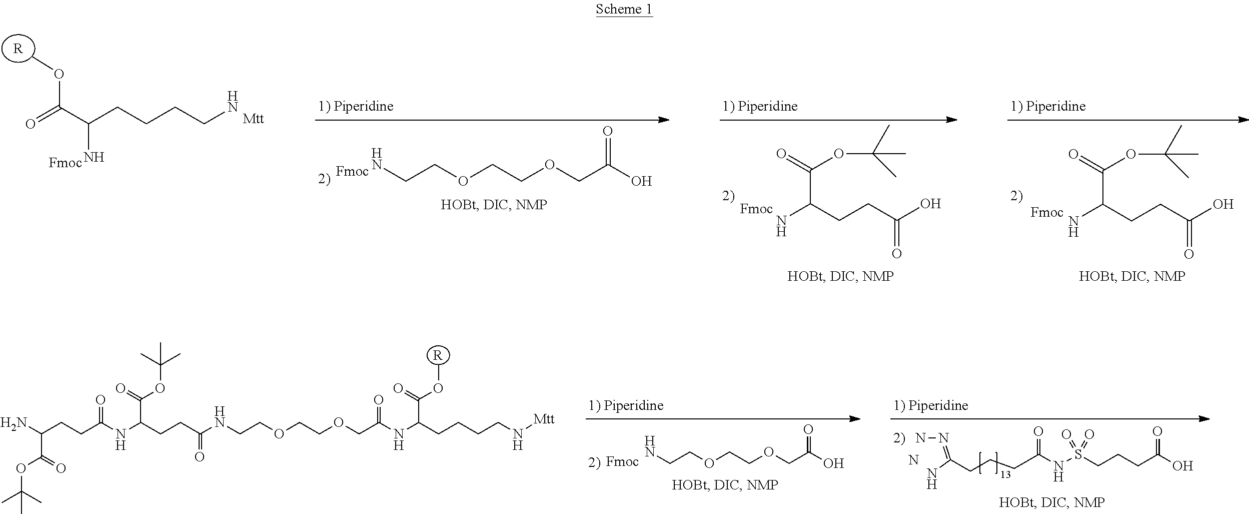

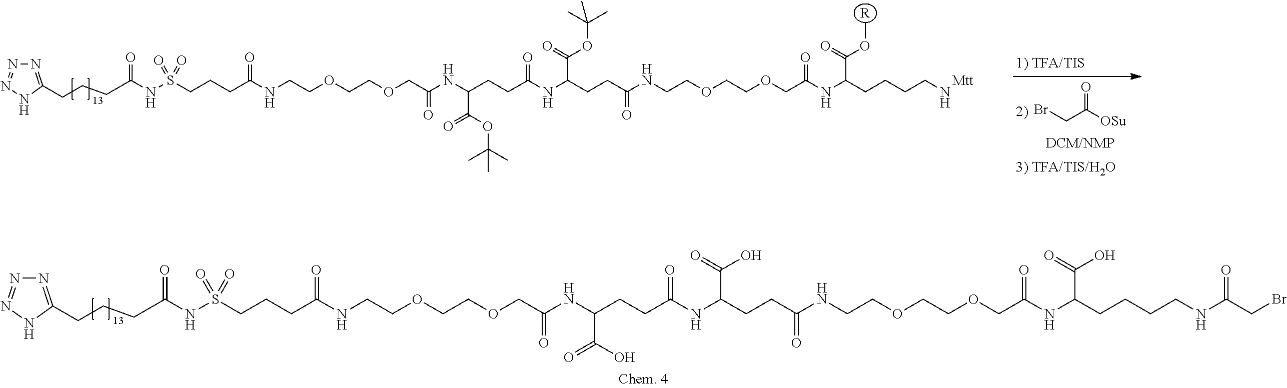

The bromoacetamide (Chem. 4a) was in the final step converted to the corresponding iodoacetamide (Chem. 5a) by halogen exchange as follow. Chem. 4a was suspended in acetone, and 5 eq. sodium iodide was added. The mixture was heated to reflux for 1 h. The reaction was then cooled on an ice bath for 15 min. Solvent was decanted off and the remaining oil solidified when washing with water. The solid material was collected and dried in vacou for 24 h giving a 45% conversion to the iodoacetamide Chem. 5a.

Iodoacetamide (Chem. 5a) can be prepared in similar way starting from the chloroacetamide (Chem 3a).

iii) Preparation of GH Receptor Antagonist with Albumin Binder at Cys101 (Compound 1; Chem. 1a):

Cystamine protected [H18D, H21N, L101C, G120R, R167N, K168A, D171S, K172R, E174S, 1179T]-hGH (21.3 mg) in 3.5 ml PBS containing 1 mM cystamine obtained as described above under i) was buffer-exchanged into 20 mM triethanolamine, 0.1 M NaCl, pH 8.5 buffer using Zeba Spin Desalting Columns (Cat. No.: 89893 from Thermo Scientific, 7 kDa cut off). A 10 mM solution of bis(p-sulfonatophenyl)phenylphosphine dihydrate dipotassium salt in 0.1 M NaCl, pH 8.5 was prepared, and 2 ml of this solution (corresponding to 20 eq.) was added to the cystamine protected polypeptide solution. The mixture was stirred for 1 h at rt. Reduction of the Cys101 mixed disulphide was confirmed by LCMS (general method). The solution was again buffer-exchanged into 20 mM triethanolamine, 0.1 M NaCl, pH 8.5 buffer using Zeba Spin Desalting Columns (Cat. No.: 89893 from Thermo Scientific, 7 kDa cut off). To the buffer exchanged solution was added albumin binder Chem. 5a as prepared under ii) above as a dry powder (6.5 mg, 5 eq.).

The clear solution was then incubated for 3 h at rt. The completion of the alkylation was confirmed by LCMS (general method). The reaction mixture was diluted to 25 ml with 20 mM triethanolamine, pH 8.5 containing 10% ethylene glycol (Buffer A). The solution was loaded onto a HiLoad 26/10 A Sepharose HP column. Unbound material was eluted with 2 bed volumes of Buffer A. The column was then eluted with a gradient of from 0-40% Buffer B (20 mM triethanolamine, 1M NaCl, 10% ethylene glycol, pH 8.5). All operations were conducted at 15° C., with a flow of 4 ml/min and monitoring the eluate at 280 nm. The fractions containing pure material were collected and concentrated by ultrafiltration (Amicon 3 KDa filters) spun at 2×20 min at 14.000 rcf. The concentrated solutions were finally buffer-exchanged into 10 mM NH₄HCO₃ using 7 kDa Zeba Spin Desalting Columns, and lyophilized to give a white powder (10.9 mg). Yield (alkylation step only) is 10.9 mg (50%). LCMS (electrospray): m/z=23206 Da.

### Example 2: GH Receptor Affinity by Surface Plasmon Resonance (SPR)

The purpose of the Example was to determine the affinity of the compound of the present invention in comparison with pegvisomant. The SPR experiments were performed using a Biacore T200 and Biacore 8K instruments (Cytiva) at 25° C. Binding kinetics were determined using a Avi-tag biotinylated version of the extracellular domain of the growth hormone receptor (hGHR-ECD) comprising hGHR residues 19-264.

For the biotinylated hGHR-ECD, the Biotin CAPture kit (Cytiva; 28920233) was used which enables reversible capture of biotinylated ligands. The chip surface was prepared by rehydration in HBS-EP buffer (0.01 M HEPES pH 7.4, 0.15 M NaCl, 3 mM EDTA, 0.005% v/v Surfactant P20) in the instrument overnight using stand-by flow. Prior to the experiment the chip surface was conditioned using Regeneration solution (3 parts of Regeneration Stock 1 (8 M guanidine-HCl) and 1 part Regeneration Stock 2 (1 M NaOH)) followed by three consecutive start-up cycles using HBS-EP buffer as analyte. Each cycle consisted of the following steps: 1) CAPture reagent (included in Biotion CAPture kit) 50 μg/mL in HBS-EP buffer (Flow rate: 2 μl/min, Contact time: 300 sec); 2) Biotinylated ligand capture (Flow rate: 10 μl/min, Contact time: 180 sec); 3) Analyte (Compound 1a, Compound 2, Somatropin and Pegvisomant) injection (Flow rate: 30 μl/min, Contact time: 120 sec, Dissociation time: 800 sec); and 4) Regeneration using Regeneration solution (Flow rate: 10 μl/min, Contact time 60 sec). The analytes were all diluted in HBS-EP buffer and applied to the ligand coated chip in concentrations of 100, 20, 4, 0.8, 0.16, 0.032 and 0 nM.

The data analysis was performed using the Biacore 8K evaluation software. Kinetic fitting was performed after baseline subtraction and curves were globally fitted using a 1:1 Langmuir binding model. K_{D} values were calculated based on the fitted association (ka) and dissociation constants (k_{d}). Results are listed in Table 1.

TABLE 1
Kinetic values determined by SPR.

| | kₐ (1/Ms) | | k_{d} (1/s) | | K_{D} (nM) | |
|---|---|---|---|---|---|---|
| | Mean | SEM | Mean | SEM | Mean | SEM |
| Compound 1a | 3.84E+05 | 3.23E+04 | 2.72E−05 | 4.13E−06 | 0.07 | 0.01 |
| Compound 2 | 6.30E+05 | 3.65E+04 | 5.23E−05 | 1.22E−05 | 0.08 | 0.02 |
| Pegvisomant | 2.49E+04 | 8.56E+03 | 5.19E−05 | 1.20E−05 | 4.04 | 1.69 |
| Somatropin | 8.42E+05 | 2.44E+05 | 1.64E−04 | 4.57E−05 | 0.35 | 0.15 |

SEM: Standard Error of the Mean (n ≥3 ).

As seen in table 1, both Compound 1a and Compound 2 have higher affinity to hGHR-ECD compared to pegvisomant.

### Example 3: GHR Binding Affinity by Isothermal Titration Calorimetry (ITC)

The purpose of the example was to measure the thermodynamic interactions between the binding partners (receptor/ligand) in solution both with and without HSA present to determine affinities.

Compound 1a, pegvisomant and Fatty acid free human serum albumin (HSA) (Sigma) were dialysed using Slide-A-Lyzer™ dialysis cassettes overnight into 4.38 mM Histidine, 2.05 mM NaCl pH 7.1 buffer. All proteins were subjected to size exclusion chromatography with multi-angle light scattering (SEC-MALS) analyses to determine the concentration before and after the up-concentration and after ITC experiments. SEC-MALS was carried out on an Agilent 1200 Series HPLC system (Agilent, Darmstadt, Germany) using a TSK G3000 SWXL column (Tosoh Bioscience, Tokyo). The column was maintained at a temperature of 20° C. and equilibrated in 122 mM Na₂HPO₄, 78 mM NaH₂PO₄, 300 mM NaCl, 4% 2-propanol, pH 6.8). Detection was done by UV absorption at 280 nm, refractive index (RI) and multi-angle laser light scattering (MALS). RI and MALS detectors were Optilab T-rEX (Wyatt) and MiniDawn Treos (LS) (Wyatt Technology, Santa Barbara, CA, USA), respectively. Data were evaluated using Chemstation B.03.01 and Astra Ver. 6.1.1.17

ITC experiments performed using a PEAQ-ITC calorimeter (Malvern, UK) at 37° C. The sample cell (300 μL) contained 10 μM Compound 1a or Pegvisomant in the presence of HSA (0, 1, 5, or 10 mg/mL). The syringe contained hGHR-ECD (ligand), at 100 μM. A thermal equilibration step was followed by a 60 sec delay and subsequently an initial 0.4-μL injection of hGHR-ECD, followed by 25 injections of 1.5 μL and a final injection of 1.1 μL at an interval of 120 sec. The stirring speed was 750 rpm, and the reference power was kept constant at 9.64 μcal/s. Data treatment done using the PEAQ-ITC analysis software (Malvern Instruments) using a one site fitting model with a fitted offset compensating. Results are shown in Table 2.

TABLE 2
Equilibrium binding parameters at increasing concentrations of HSA using ITC.

| | | | | | ΔH | | ΔG | | TΔS | |
|---|---|---|---|---|---|---|---|---|---|---|
| [HSA] | N (sites) | | K_{D} (nM) | | (kcal/mol) | | (kcal/mol) | | (kcal/mol) | |
| mg/mL | Mean | SEM | Mean | SEM | Mean | SEM | Mean | SEM | Mean | SEM |

Compound 1a

| | | | | | | | | | | |
|---|---|---|---|---|---|---|---|---|---|---|
| 0 | 1.09 | 0.05 | 2.1 | 0.3 | −32.9 | 1.1 | −12.3 | 0.1 | 20.6 | 1.1 |
| 1 | 1.10 | 0.05 | 4.8 | 0.2 | −30.3 | 0.7 | −11.8 | 0.0 | 18.5 | 0.7 |
| 5 | 1.11 | 0.05 | 4.9 | 0.7 | −29.6 | 0.6 | −11.8 | 0.1 | 17.7 | 0.6 |
| 10 | 1.11 | 0.06 | 3.5 | 0.2 | −29.4 | 0.9 | −12.0 | 0.1 | 17.4 | 0.9 |

Pegvisomant

| | | | | | | | | | | |
|---|---|---|---|---|---|---|---|---|---|---|
| 0 | 1.23 | 0.15 | 6.6 | 0.9 | −31.2 | 0.7 | −11.6 | 0.1 | 19.6 | 0.8 |
| 1 | 1.17 | 0.09 | 6.6 | 1.3 | −31.7 | 1.0 | −11.6 | 0.1 | 20.1 | 1.1 |
| 5 | 1.24 | 0.12 | 5.7 | 0.6 | −32.3 | 0.5 | −11.7 | 0.1 | 20.6 | 0.6 |
| 10 | 1.21 | 0.08 | 4.8 | 1.2 | −31.5 | 0.9 | −11.9 | 0.2 | 19.7 | 1.0 |

SEM: Standard Error of the Mean (n = 3)

N is binding stoichiometry, K_{D} is dissociation constant, ΔH is change in enthalpy, ΔG is change in Gibb's free energy, ΔS is change in entropy, and T is temperature.

It is seen that K_{D} for compound 1a is lower than pegvisomant, thus, compound 1a also shows higher affinity in the ITC assay. It is also seen that for both compound 1a and pegvisomant, the affinity does not change significant in the presence of HSA as compared to 0 mg/mL HSA.

### Example 4: In Vitro Human Growth Hormone Receptor Activity Assay

The purpose of the example was to determine activity of selected compounds as to whether they act as agonists or antagonists at the hGHR.

To evaluate the in vitro activity of human growth hormone receptor (hGHR) agonists or antagonists, they were tested for their ability to induce phosphorylation of STAT3 in reporter cells expressing hGHR. To produce the reporter cell line, BHK-21 cells (ATCC® CCL-10™) were stably transfected with a reporter plasmid encoding a STAT3 response element coupled to luciferase and an expression plasmid encoding hGHR. A single cell clone was isolated for the reporter cell line. When reporter cells are exposed to a hGHR agonist, hGHR cause phosphorylation of the transcription factor STAT3. Phosphorylated STAT3 causes activation of transcription at the STAT3 response element and luciferase is produced. Luciferase is able to convert luciferin to oxyluciferin, a process that produces bioluminescence. Upon cell lysis and supply of luciferase substrate, luciferase activity can be quantified by detection of luminescence. Therefore, addition of a hGHR agonist and subsequent addition of detection reagent to reporter cells causes generation of luminescence in a dose dependent manner. This procedure is below referred to as agonist mode. Similarly, addition of a hGHR antagonist, followed by addition of a hGHR agonist and detection reagent causes generation of luminescence in an inverse dose dependent manner. This procedure is below referred to as antagonist mode. By testing a number of different concentrations of a hGHR agonist or antagonist, a half maximal effective concentration, referred to as EC₅₀ in agonist mode and IC₅₀ in antagonist mode, can be determined. These measures are reported for all compounds tested. In all the in vitro hGHR activity experiments, somatropin was used as a positive control for hGHR agonism and Pegvisomant was used as a positive control for hGHR antagonism.

To perform the assay, BHK21/GHR/STAT3-RE clone 9 cells were thawed and suspended in growth medium (DMEM (Gibco 31966-021) containing 10% FBS (Gibco 10091-148) and 1% P/S (Lonza DE17-602E)). Cells were then seeded in 25 μL/well at 20,000 cells/well in white opaque 384-well plates (Greiner 781080) overnight. The following day, test compounds were serial diluted in assay medium (DMEM without phenol red containing 1% OVA (Sigma A5505), 0.01% Tween20 (Roche 33766700) and 1% P/S). The cell plates were emptied and 20 μL/well of diluted test compounds were added to the cells in quadruplicates. For agonist mode, 5 L/well assay medium was immediately added to half of the cell plates. For antagonist mode, 5 μL/well 5 nM somatropin was added to the other half of the cell plates after 30 min of incubation at 37° C. and 5% CO₂. Following an additional 4 hours of incubation at 37° C. and 5% CO₂, 12.5 μL/well detection reagent (Steady-GLO, Promega E2550) was added to all the cell plates and the plates were incubated in the dark for 15 min at rt. Thereafter, luminescence was detected by an EnVision Multimode Plate Reader (PerkinElmer). To determine the EC₅₀ and IC₅₀ of each test compound, a four-parameter logistic regression was done on the raw data for each test compound using the Python package SciPy optimize. Each test compound was tested in a minimum of two independent experiments. The reported values are averages±standard error of the mean across all experiments. Results are shown in Table 3.

TABLE 3
hGHR STAT3 activity assay.

| | Agonist mode | Antagonist mode |
|---|---|---|
| Compound | EC₅₀ (nM) | IC₅₀ (nM) |
| Somatropin | 0.13 ± 0.01 | ND |
| Pegvisomant | ND | 256 ± 113 |
| Compound 2 | ND | 9 ± 5 |
| Compound 1a | ND | 74 ± 1 |

ND: No response seen.

The agonist data shows as expected that somatropin is an agonist at the hGHR. In addition, it is seen that none of pegvisomant, compound 1a and compound 2 shows measurable agonist activity. In antagonist mode, it is seen that all of pegvisomant, compound 1a and compound 2 show antagonist activity, however, compounds 1a and compound 2 show improved antagonist activity as compared to known antagonist pegvisomant.

### Example 5: BAF-3 GHR Assay to Determine GHR Activity

The purpose of the example was to determine activity of selected compounds as to whether they act as agonists or antagonists at the hGHR.

The BAF-3 cells (a murine pro-B lymphoid cell line derived from the bone marrow) were originally IL-3 dependent for growth and survival. IL-3 activates JAK-2 and STAT which are the same mediators as GH is activating upon stimulation. After transfection of the human growth hormone receptor the cell line was turned into a growth hormone-dependent cell line. This clone can be used to evaluate the effect of different growth hormone samples on the survival of the BAF-3GHR. (Culture medium: RPMI 1640+GlutaMAX™+FBS+hGH).

The BAF-3GHR cells were grown in starvation medium (culture medium without growth hormone) for 24 h at 37° C., 5% CO₂. The cells are then used to test either growth hormone receptor agonists or antagonists.

In the agonist mode, the cells were washed and re-suspended in starvation medium and seeded in plates. 10 μL of test compound or control (human growth hormone in agonist mode) in different concentrations was added to the cells, and the plates were incubated for 68 h at 37° C., 5% CO₂.

In the antagonist mode, the cells were washed and re-suspended in starvation medium and seeded in plates. 10 μL of a 2 nM growth hormone solution was added to the cells followed by 10 μL of test compound or control (pegvisomant) in different concentrations was added to the cells. The plates were then incubated for 68 h at 37° C., 5% CO₂.

AlamarBlue® was added to each well and the cells were incubated for another 4h. AlamarBlue® is a redox indicator and is reduced by reactions innate to cellular metabolism and, therefore, provides an indirect measure of viable cell number.

The metabolic activity of the cells was measured in a fluorescence plate reader. The fluorescence was measured in the plate reader at excitation 544 nm and emission 590 nm with shake before read and 9-point pr. well in a cross pattern. From the concentration-response curves the activity (amount of a compound that stimulates/inhibits the cells with 50%) was calculated.

Three compounds, Pegvisomant, Compound 2 and Compound 1a were tested in the assay in the agonistic mode with somatropin as positive control. The results are shown in Table 4.

Pegvisomant, Compound 2 and Compound 1a were tested in antagonistic assay mode with pegvisomant as positive control. The results are shown in Table 4.

TABLE 4
BAF-3 GHR activity assay, agonist, and antagonist mode.

| | Agonist mode | Antagonist mode |
|---|---|---|
| Compound | EC₅₀ (nM) | IC₅₀ (nM) |
| Somatropin (hGH) | 0.0654 | — |
| Pegvisomant | ND | 205 |
| Compound 2 | ND | 1.39 |
| Compound 1a | ND | 18.6 |

ND: No response seen.

Data in Table 4, agonist mode, confirms agonism for somatropin with an estimated EC₅₀ of 0.0654 nM, whereas no concentration-response was obtained for the other test compounds showing that neither of them display agonistic properties in the current assay.

The IC₅₀ values (antagonist mode) shows, as expected, that pegvisomant have antagonistic properties with an IC₅₀ of 205 nM. It is also seen that Compound 2 can inhibit BAF cell proliferation in the presence of somatropin and had a 147-fold higher antagonistic potency compared to pegvisomant, whereas Compound 1a had a 11-fold higher antagonistic potency compared to pegvisomant. Thus, it is confirmed that both compounds 1a and 2 have improved antagonistic potency as compared to pegvisomant.

### Example 6: Rat PK Data—Compound 1a

The purpose of the example was to investigate the pharmacokinetic properties of compound 1a in Sprague-Dawley rats after single dose i.v. and s.c. administration.

Twenty-four male Sprague-Dawley rats of approximately 250 g were included in the study. The animals were acclimatised for one week before entry into the study. During the experiment the animals were kept and handled according to standard procedures in the Novo Nordisk Animal Unit and allowed free access to food and water. All rats were weighed before the start of the study and on the day of dosing immediately prior to dosing. The animals were divided into two treatment groups, one with i.v. dosing and one with s.c. dosing.

Compound 1a was dissolved to a final concentration of 450 nmol/L in a standard buffer, consisting of: pH=7.4; 8.05 mM sodium phosphate dibasic; 1.96 mM potassium dihydrogen phosphate; 140 mM sodium chloride; 0.007% polysorbate 20.

At the start of the experiment all animals received a single dose of 180 nmol/kg of compound 1a either as a single dose i.v. bolus injection in the tail vein (i.v. treatment group) or a single s.c. injections in the neck (s.c. treatment group).

Blood samples were drawn from all animals from the tail vein. 0.1 ml blood was drawn according to the following schedule:
i.v. treatment group: Pre-dose, 0.08, 0.5, 1, 2, 4, 8, 18, 24, 30, 48, 72, 96, 168 and 240 hours post dosing.
s.c. treatment group: Pre-dose, 0.25, 0.5, 1, 2, 4, 8, 18, 24, 30, 48, 72, 96, 168 and 240 hours post dosing.

Blood sampling was conducted as a sparse sampling schedule, where both treatment groups were subdivided into 4 groups of 3 rats ensuring that blood was drawn from 3 animals at each time point per treatment group and each animal was bled 3-4 times during the entire study.

At each sampling time point 0.1 ml (8-10 droplets) of blood was collected from the tail vein and transferred to Eppendorf tubes containing 8 mM EDTA. After gentle mixing the blood sample was kept on ice until centrifugation within 10 minutes at 4000 g for 5 minutes at +5° C. After centrifugation the plasma fraction was divided into two fractions and analysed for test compound concentration and IGF-1 concentration as described below.

### Plasma Analysis of IGF-1 in Rat Plasma:

Samples were analysed for rat IGF-1 content using an internally developed homogenous Luminescence Oxygen Channeling Immuno assay (LOCI). Before performing the analysis, samples have been pre-treated to release IGF-1 from their binding proteins in plasma. During the assay, a concentration dependent bead-analyte-immune complex was created, resulting in light output which was measured on a Perkin Elmer Envision reader. In the assay, (anti rat IGF-1) mAb 1212-0362-conjugated acceptor-beads and biotinylated mAb 1212-0307 (also raised against rat IGF-1) were used together with generic streptavidin-coated donor beads. The lower limit of quantification (LLOQ) is 20 ng/ml.

### Plasma Analysis of Compound 1a in Rat Plasma:

Samples were analysed for Compound 1a content using an internally developed homogenous Luminescence Oxygen Channeling Immuno assay (LOCI). During the assay, a concentration dependent bead-analyte-immune complex was created, resulting in light output which was measured on a Perkin Elmer Envision reader. In the assay, growth hormone binding protein-conjugated acceptor-beads and biotinylated pAb 20GS10 (raised against human growth hormone) were used together with generic streptavidin-coated donor beads. The lower limit of quantification (LLOQ) is 50 μM.

Data from the analyses are shown in Table 5.

TABLE 5
Concentrations at given time of Compound 1a
following i.v. and s.c. administration.

Mean concentration (nM)

| Time (h) | i.v. | s.c. |
|---|---|---|
| 0.08 | 3207 | — |
| 0.25 | — | 31.0 |
| 0.5 | 2693 | 63.5 |
| 1 | 2540 | 181 |
| 2 | 1997 | 237 |
| 4 | 1750 | 384 |
| 7 | 1347 | 552 |
| 18 | 739 | 545 |
| 24 | 488 | 437 |
| 30 | 501 | 440 |
| 48 | 70.9 | 74.4 |
| 72 | 22.6 | 27.3 |
| 96 | 4.77 | 6.93 |
| 168 | 0.260 | 0.205 |

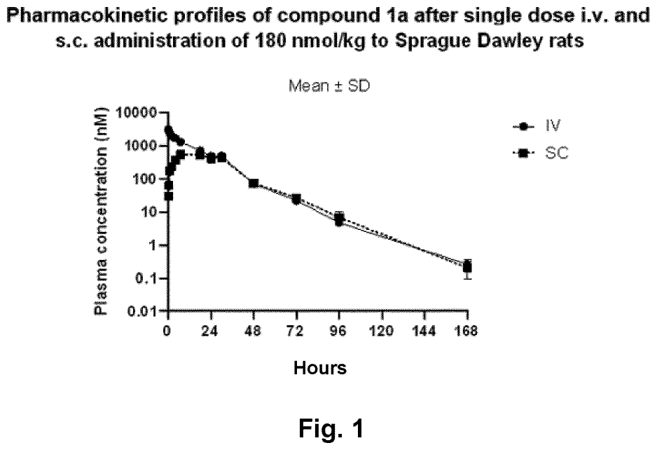

The plasma concentration-time profiles of Compound 1a after i.v. and s.c. administration is shown in the FIG. 1 and shows that the s.c. absorption was completed 24-48 hours after dosing and that compound 1a did not show absorption-limited elimination.

Plasma concentration-time data was analysed by non-compartmental pharmacokinetics using Phoenix WinNonlin 6.4 (Pharsight Corporation). Calculations were performed using mean concentration-time values from three animals at each time point. The pharmacokinetic parameter estimates are shown in Table 6 below:

TABLE 6
Pharmacokinetic parameters of compound 1a
following i.v. or s.c. administration.

Treatment group

| Parameter | Unit | i.v. | s.c. |
|---|---|---|---|
| C₀ | (nmol/L) | 3320 | — |
| tₘₐₓ | (h) | — | 7 |
| Cₘₐₓ | (nmol/L) | 3210 | 552 |
| AUCₗₐₛₜ | (h*nmol/L) | 38400 | 20500 |
| AUC | (h*nmol/L) | 38400 | 20500 |
| Clearance | (L/h/kg) | 0.00469 | — |
| V_{z} | (L/kg) | 0.101 | — |
| t_{1/2} | (h) | 14.9 | 16.4 |
| MRT | (h) | 15.4 | 24.2 |
| F | — | — | 0.53 |

Table 6 shows the key pharmacokinetic parameters for compound 1a. Co is the concentration of compound 1a immediately after i.v. injection. tₘₐₓ is the maximum plasma concentration of compound 1a after s.c. administration and estimated to 7h. Cₘₐₓ is the maximum concentration of compound 1a in the blood and estimated to 3210 and 552 nmol/L following i.v. and s.c. administration, respectively. AUCₗₐₛₜ and AUC are the area under the curve at the last data point and estimated at infinity, respectively, and is a measure of the total drug exposure over time. Using the AUC values from s.c. and i.v., the absolute bioavailability (F) can be calculated. Clearance is calculated from dose/AUC. V_{z} is volume of distribution, and t_{1/2} is the terminal half-life of compound 1a after the absorption phase has been completed. MRT is the mean residence time of compound 1a corresponding to the average time compound 1a is in the body and thus, s.c. administration results prolonged action.

### Example 7: Dog PK Data—Compound 1a

The purpose of the example was to investigate the pharmacokinetic properties of Compound 1a and measure the IGF-1 concentrations in Beagle dogs after single dose i.v. administration of two doses, 10 mg/kg and 3 mg/kg.

Nine beagle dogs, male, average weight ~11.5 kg, were divided into 3 groups as below: vehicle, Compound 1a at 3 mg/kg, and Compound 1a at 10 mg/kg, n=3 per group. Dose volume was 0.3 ml/kg for each dog at given concentration of injection solution. All animals were fasted for ~20 hr prior to experiment. Following i.v. bolus injection, serial blood samples were collected until 504 hr post-dosing for quantification of Compound 1a and circulating IGF-1. Detailed blood sampling time point were: −5 min (pre-dose), 5, 15, 30, 45 min, 1, 1.5, 2, 4, 7, 10 h, 24, 30, 48, 72, 120, 168, 216, 264, 312, 360, 432, 504 hr.

For every single time point, 1.2 ml blood was collected in EDTA-coated tubes, followed by 3-4 times of gentle inversion. The blood samples were kept on ice until centrifugation (4 min, 4° C., 4000 rpm).

One hundred μl ×2 plasma was transferred immediately into labelled Micronic tubes and kept at −20° C. until assay. Another 200 μl set of back-up samples will be stored for the potential re-test (−20° C. or −80° C.).

### Plasma Analysis of IGF-1 in Dog Plasma:

Concentration of the IGF-1 in dog plasma samples was determined using a commercial Enzyme-Linked Immunosorbent Assay (ELISA) kit (Mediagnost, product No.: E20). The calibrators and controls were prepared by reconstitute “CAL” and “Control” in 500 μL sample buffer (SB). The dynamic range of the assay was between 2 to 50 ng/ml. The lower limit of quantification (LLOQ) of the assay was 0.091 ng/ml. Controls were 1:20 diluted in the sample buffer PP (BUF SB). The dog plasma sample were 1:15 diluted in SB. The dilution procedures were performed on the apricot automated liquid handling system. Eighty μL antibody conjugate “Ab” was transferred to the 96-well assay plates. Twenty μL diluted sample/calibrator/control were applied to assay plates and incubated for 1 hour at room temperature (RT) with 350 rpm shaking. Assay plates were washed 5 times with washing buffer (WP). One hundred μL enzyme conjugate (CONJ) was applied to assay plates and incubate for 1 hour at RT with 350 rpm shaking. Assay plates were washed 5 times with WP. One hundred μL substrate solution (SUBST) was applied to assay plates and incubate in dark for 15 minutes at RT. 100 μL stop solution (SL) were added to assay plates. Plates were read in an Tecan plate reader at 450 nm. Calibrator curves were generated by fitting signals and concentrations to a 5-parameter logistic model with standards weighting as 1/signal². The concentrations of the controls and IGF-1 were calculated with the calibrator curve and corrected for dilution. The recoveries of the controls are within 80%-120%. The dilution recoveries of the samples are within 80%-120%. The coefficient of variations (CV) between replicates are within ±10%.

Data from the analyses are shown in Table 7.

TABLE 7
Concentrations (Mean ± SD) at given time of IGF-1 following
i.v. administration of 3 mg/kg and 10 mg/kg respectively.

IGF-1 concentration (ng/mL)

| Time (h) | 3 mg/kg | 10 mg/kg |
|---|---|---|
| Predose | 137 ± 24.5 | 110 ± 40.9 |
| 0.08 | 136 ± 21.2 | 111 ± 36.7 |
| 0.25 | 137 ± 19.9 | 110 ± 40.8 |
| 0.5 | 131 ± 18.0 | 105 ± 41.3 |
| 0.75 | 130 ± 16.2 | 106 ± 38.8 |
| 1 | 132 ± 18.9 | 108 ± 40.2 |
| 1.5 | 132 ± 18.2 | 107 ± 39.1 |
| 2 | 133 ± 18.3 | 110 ± 43.2 |
| 4 | 131 ± 16.2 | 111 ± 42.0 |
| 7 | 140 ± 14.6 | 110 ± 37.9 |
| 10 | 138 ± 15.7 | 112 ± 41.5 |
| 24 | 113 ± 11.0 | 91.8 ± 35.9 |
| 30 | 104 ± 11.5 | 86.0 ± 35.7 |
| 48 | 86.5 ± 10.0 | 70.5 ± 28.1 |
| 72 | 72.9 ± 11.0 | 54.2 ± 22.8 |
| 120 | 54.2 ± 13.6 | 36.4 ± 14.5 |
| 168 | 50.8 ± 21.5 | 30.0 ± 13.6 |
| 216 | 49.2 ± 23.1 | 26.5 ± 8.76 |
| 264 | 51.5 ± 21.4 | 25.9 ± 10.6 |
| 312 | 63.1 ± 27.1 | 27.8 ± 16.6 |
| 360 | 100 ± 17.6 | 26.7 ± 16.3 |
| 432 | 133 ± 23.1 | 53.9 ± 41.0 |
| 504 | 134 ± 27.4 | 79.2 ± 45.4 |

### Plasma Analysis of Compound 1a in Dog Plasma:

Concentration of Compound 1a in dog plasma samples was determined using Luminescence Oxygen Channelling Immunoassay (LOCI). The calibrators and controls were prepared by manually spiking the analyte into dog plasma. The dynamic range of the assay was between 4.9 to 20000 μM. The lower limit of quantification (LLOQ) of the assay was 4 pM. The three controls were 3000, 100, and 4 μM respectively. The dog plasma sample dilutions were performed on the Bravo automated liquid handling system.2 μL diluted sample/calibrator/control is applied together with 3 μl sample buffer in 384-well LOCI plates, incubation with mAb (NNC1212-0000-0575) conjugated acceptor-beads and biotinylated growth hormone binding protein at 22° C. overnight. Thirty μL streptavidin coated donor beads (67 μg/ml) were added to each well and incubated for 30 minutes at 20° C. The plates were read in an Ensight plate reader at 21-22° C. with a filter having a bandwidth of 520-645 nm after excitation by a 680 nm laser. The total measurement time per well was 210 ms including a 70 ms excitation time. Calibrator curve was generated by fitting signals and concentrations to a 5-parameter logistic model with standards weighting as 1/signal². The concentrations of the controls and Compound 1a were calculated with the calibrator curve and corrected for dilution. The recoveries of the controls were within 80%-120%. The dilution recoveries of the samples were within 80%-120%. The coefficient of variations (cv) between replicates were within ±10%.

Data from the analyses are shown in Table 8.

TABLE 8
Concentrations (Mean ± SD) at given time of Compound 1a
following i.v. administration of 3 mg/kg and 10 mg/kg respectively.

Compound 1a concentration (nM)

| Time (h) | 3 mg/kg | 10 mg/kg |
|---|---|---|
| 0.08 | 2467 ± 90.7 | 8600 ± 311 |
| 0.25 | 2460 ± 151 | 8443 ± 121 |
| 0.5 | 2260 ± 123 | 7530 ± 212 |
| 0.75 | 2213 ± 170 | 7633 ± 211 |
| 1 | 2160 ± 85.4 | 7500 ± 159 |
| 1.5 | 2137 ± 37.9 | 7093 ± 415 |
| 2 | 2150 ± 26.5 | 7203 ± 307 |
| 4 | 2100 ± 43.6 | 7423 ± 188 |
| 7 | 1953 ± 140 | 6633 ± 315 |
| 10 | 1773 ± 64.3 | 6347 ± 401 |
| 24 | 1253 ± 70.9 | 4667 ± 809 |
| 30 | 1163 ± 41.6 | 4023 ± 192 |
| 48 | 922 ± 37.5 | 3347 ± 405 |
| 72 | 779 ± 85.4 | 2600 ± 72.1 |
| 120 | 515 ± 44.6 | 1867 ± 289 |
| 168 | 366 ± 93.1 | 1343 ± 101 |
| 216 | 233 ± 62.9 | 930 ± 52.5 |
| 264 | 132 ± 51.0 | 614 ± 69.7 |
| 312 | 25.8 ± 28.8 | 415 ± 43.4 |
| 360 | 0.473 ± 0.358 | 255 ± 32.2 |
| 432 | — | 74.3 ± 63.8 |
| 504 | — | 4.61 ± 7.53 |

Plasma concentration-time data was analysed by non-compartmental pharmacokinetics using Phoenix WinNonlin 6.4 (Pharsight Corporation). Calculations were performed using mean concentration-time values from three animals at each time point. The pharmacokinetic parameter estimates are shown in Table 9 below:

TABLE 9
Pharmacokinetic parameters of compound 1a following i.v.
administration of 3 mg/kg and 10 mg/kg respectively.

| | | Dosing group | |
|---|---|---|---|
| Parameter | Unit | 3 mg/kg | 10 mg/kg |
| C₀ | (nmol/L) | 2467 ± 90.7 | 8593 ± 220 |
| AUCₗₐₛₜ | (h*nmol/L) | 165034 ± 15870 | 573414 ± 54772 |
| AUC | (h*nmol/L) | 171276 ± 20283 | 582613 ± 53120 |
| Clearance | (L/h/kg) | 0.00075 ± 0.000084 | 0.00074 ± 0.000066 |
| V_{z} | (L/kg) | 0.07636 ± 0.00715 | 0.08486 ± 0.00658 |
| t_{1/2} | (h) | 70.1 ± 13.7 | 79.3 ± 4.25 |
| MRT | (h) | 98.4 ± 18.6 | 106 ± 6.28 |

Table 9 shows the key pharmacokinetic parameters for compound 1a. CO is the concentration of compound 1a immediately after i.v. injection. AUClast and AUC are the area under the curve at the last data point and estimated at infinity, respectively, and is a measure of the total drug exposure over time. Clearance is calculated from dose/AUC. Vz is volume of distribution, and t_{1/2} is the terminal half-life of compound 1a. MRT is the mean residence time of compound 1a corresponding to the average time compound 1a is in the body.

### Example 8: In Vitro Binding of Compound 1a to Proteins in Plasma from Mice, Rats, Rabbits, Dogs, and Humans

The aim was to use the equilibrium shift assay technology to investigate binding between Compound 1a and plasma proteins in free solution. A kinetic analysis was performed to determine the percentage fraction unbound of Compound 1a in plasma.

Fatty acid free human serum albumin (HSA) was immobilised to the Mini-Leak beads according to the procedure described by Kurtzhals et al. (1, 2). In brief, the storage media was removed from the Sepharose beads by suction on a filter. Afterwards the beads were washed with three volumes of isotonic sodium chloride. To one gram of Sepharose beads, 2 ml of 5% (w/v) HSA in Milli-Q water and 2 ml of 30% (w/v) PEG20000 in 0.3 M HCO3, were added. The solution was mixed on a Hula-Shaker overnight. Subsequently, the assay medium was removed by suction on a filter and the beads were washed with 20 ml of isotonic sodium chloride. The beads were then treated with 0.2 M ethanolamine for five hours. Afterwards, the beads were washed with 12 ml of phosphate buffer (pH 11, three times) and 12 ml of glycine buffer (pH 3, three times). The beads were stored in PBS buffer (pH 7.2) at a concentration of 200 mg beads per ml. The concentration of immobilised HSA was determined according to the procedure reported by Kurtzhals et al. (1).

If a drug (D) is incubated with an immobilised protein (Pᵢₘₘ) and a soluble protein (Pₛₒₗ), two binding reactions can occur:

$$D + {P}_{imm} ↔ {DP}_{imm} \qquad 1 )$$

$$D + {P}_{sol} ↔ {DP}_{sol} \qquad 2 )$$

When the concentration of drug is much lower than the concentration of protein, it is expected that the protein only binds one molecule of drug. At equilibrium, the association constants for the two binding reactions are:

$${K}_{a , imm} = \frac{{[ DP ]}_{imm}}{{[ D ] [ P ]}_{imm}} \qquad 1 )$$

$${K}_{a , sol} = \frac{{[ DP ]}_{sol}}{{[ D ] [ P ]}_{sol}} \qquad 2 )$$

K_{a,sol} can be rearranged and put in the equation for K_{a,sol}, which gives following equations

$${K}_{a , sol} = \frac{{[ DP ]}_{sol}}{{[ D ] [ P ]}_{sol}} \to [ D ] = \frac{{[ DP ]}_{sol}}{{{K}_{a , sol} [ P ]}_{sol}}$$

$${K}_{a , imm} = \frac{{[ DP ]}_{imm}}{{[ D ] [ P ]}_{imm}} = \frac{{[ DP ]}_{imm}}{{\frac{{[ DP ]}_{sol}}{{{K}_{a , sol} [ P ]}_{sol}} [ P ]}_{imm}}$$

$$⇓$$

$$\frac{{[ DP ]}_{sol}}{{[ DP ]}_{imm}} = \frac{{{K}_{a , sol} [ P ]}_{sol}}{{{K}_{a , imm} [ P ]}_{imm}}$$

The concentration of immobilised HSA can be estimated by plotting DPₛₒₗ/DPᵢₘₘ versus the concentration of soluble HSA. Assuming that K_{a,sol}=K_{a,imm}, the slope of the linear plot is 1/[HSAᵢₘₘ].

The concentration of immobilised HSA can be determined incubating of fixed amount of beads with various concentrations of soluble HSA. Herein, 15.5 mg of beads were incubated with five different concentrations of soluble HSA (final concentration of HSA is: 2.5, 5, 10, 20 and 40 μM) and 250 nM of a reference compound in PBS buffer (pH 7.2) for two hours at room temperature. After incubation, the samples were centrifuged at 750 g for 10 min. Samples were then precipitated and subsequently analysed by LC-MS.

The linear correlation between the fraction of unbound and bound (fᵤ/f_{b}) and the concentration of soluble HSA can be plotted, and the slope used to calculate the concentration of immobilized HSA per mg Mini-Leak beads as follows.

$${HSA}_{imm} = \frac{{n}_{HSAimm}}{{m}_{beads}} = \frac{[ {HSA}_{imm} ] \times {V}_{incubation}}{{m}_{beads}} = \frac{\frac{1}{slope} \times {V}_{incubation}}{{m}_{beads}} = \frac{\frac{1}{slope} \times 0.0005 L}{15.5 mg} = \frac{nmol}{mg beads}$$

The average of 12 experiments were used, resulting in a concentration of 0.51 nmol of immobilized HSA per mg beads. This value was used for the calculation of K_{d,HSAimm} and the unbound fraction in plasma.

The binding between a drug (A) and a protein (P) can be described as:

$$A + P ⇆ AP$$

Due to the law of mass conservation the binding between a drug (A) and a protein (P) can be described with the dissociation constant as defined:

$${K}_{d} = \frac{[ A ] [ P ]}{[ AP ]}$$

At equilibrium the concentration of drug (A) and drug-protein (AP) can be described as:

$$[ A ] = {c}_{0} * {f}_{u} = ( [ A ] + [ AP ] ) * {f}_{u}$$

$$and$$

$$[ AP ] = {c}_{0} * {f}_{b} = ( [ A ] + [ AP ] ) * {f}_{b}$$

Hence the formula for the dissociation constant can be rearranged:

$${K}_{d} = \frac{[ A ] [ P ]}{[ AP ]} = \frac{( ( [ A ] + [ AP ] ) * {f}_{u} ) * [ P ]}{( [ A ] + [ AP ] ) * {f}_{b}}$$

$$↓$$

$$\frac{( [ A ] + [ AP ] ) * {f}_{u}}{( [ A ] + [ AP ] ) * {f}_{b}} = \frac{1}{[ P ]} * {K}_{d}$$

$$↓$$

$$\frac{{f}_{b}}{{f}_{u}} = \frac{1}{{K}_{d}} * [ P ]$$

By this formula the affinity towards immobilised HSA can be calculated by plotting the ratio between bound and unbound compound (f_{b}/fᵤ) against the concentration of immobilised albumin.

Experimentally, this was conducted by incubating a final concentration of 250 nM of Compound 1a six different concentrations of immobilised HSA in PBS buffer (pH 7.2) and two reference incubations without immobilised HSA. In addition, a final concentration of 0.01% P188 was used to prevent adsorption to the vessels. The samples were incubated for two hours at 37° C. under mechanical shaking on a KingFisher robot (Thermo Fisher Scientific) using 1% OVA coated deep-well KingFisher plates and tip combs. After incubation, the samples were centrifuged for 10 min at 750 g to sediment the beads. Prior to sample preparation, one volume of supernatant was diluted with one volume of blank plasma. The mixtures were assayed by LC-MS for Compound 1a.

The unbound and bound fraction was calculated using following equations:

$${f}_{b} = \frac{{[ 0559 - 0000 - 1657 ]}_{ref ( mean )} - {[ 0559 - 0000 - 1657 ]}_{beads}}{{[ 0559 - 0000 - 1657 ]}_{ref ( mean )}}$$

$${f}_{u} = 1 - {f}_{b}$$

The average of the concentration in the two reference incubations was used for calculation of f_{b}. By plotting f_{b}/fᵤ against the concentration of immobilised HSA a straight line is obtained, where the slope (α) can be converted to the dissociation constant by this formula:

$$α = \frac{1}{{K}_{d , HSAimm}} \to {K}_{d , HSAimm} = \frac{1}{α}$$

The individual determination of K_{d,HSAimm} is based on eight data points. In each calculation, one of the reference data points and another data point could be excluded from the calculation.

The free fraction can be described as:

$${f}_{u} = \frac{1}{1 + \sum \frac{{P}_{i}}{{K}_{d}^{i}}}$$

By this formula, the free fraction when incubating with plasma and immobilised HSA can be expressed as:

$${f}_{u} = \frac{1}{1 + \frac{[ HSA ]}{{K}_{d , HSAimm}} + \frac{[ P ]}{{K}_{d , plasma}}}$$

The free concentration (A) is:

$$A = {c}_{0} * {f}_{u}$$

The total bound concentration (e.g., bound to beads (AB) and bound to plasma (AP)) is defined as

$$AB + AP = {c}_{0} * {f}_{b}$$

After removing the beads, the concentration in the supernatant (APA) is the concentration bound to plasma proteins (AP) and free concentration (A):

$$APA = AP + A$$

The concentration of AP can be defined as:

$$AP = ϑ * {c}_{0} * {f}_{b}$$

Where 29 is ratio between compound bound to plasma proteins ([AP]) and total amount bound to plasma proteins and immobilised HSA ([AB]). This ratio can be expressed as follows:

$$ϑ = \frac{[ AP ]}{[ AP ] + [ AB ]} = \frac{\frac{[ A ] [ P ]}{{K}_{d , plasma}}}{\frac{[ A ] [ P ]}{{K}_{d , plasma}} + \frac{[ A ] [ HSA ]}{{K}_{d , HSAimm}}} = \frac{\frac{[ P ]}{{K}_{d , plasma}}}{\frac{[ P ]}{{K}_{d , plasma}} + \frac{[ HSA ]}{{K}_{d , HSAimm}}}$$

The concentration in the supernatant (APA) can be rearranged.

$$APA = ϑ * {c}_{0} * {f}_{b} + {c}_{0} * {f}_{u} = ( \frac{\frac{[ P ]}{{K}_{d , plasma}}}{\frac{[ P ]}{{K}_{d , plasma}} + \frac{[ HSA ]}{{K}_{d , HSAimm}}} ) * {c}_{0} * ( 1 - \frac{1}{1 + \frac{[ HSA ]}{{K}_{d , HSAimm}} + \frac{[ P ]}{{K}_{d , plasma}}} ) + {c}_{0} * ( \frac{1}{1 + \frac{[ HSA ]}{{K}_{d , HSAimm}} + \frac{[ P ]}{{K}_{d , plasma}}} )$$

The above equation can then be rearranged:

$$APA = \frac{{c}_{0} * {K}_{d , HSAimm} * ( α * P + {K}_{d , plasma} )}{[ {HSA}_{imm} ] * {K}_{d , plasma} + {K}_{d , HSAimm} * ( α * P + {K}_{d , plasma} )}$$

Where:
APA: Concentration of compound in supernatant (free and bound to plasma proteins).
c₀: Initial concentration of test compound.
P: Concentration of protein (arbitrarily set to 600 μM).
K_{d,HSAimm}: Dissociation constant from HSA immobilised on the Mini-Leak beads.
K_{d,plasma}: Dissociation constant from proteins in plasma.
α: Plasma dilution factor.
[HSA]: Concentration of HSA immobilised on the Mini-Leak beads.

The pseudo-dissociation constant towards plasma proteins can be estimated using a value, which gives the lowest sum of squared residuals between the measured APA values and theoretic calculated APA values.

Using the arbitrary concentration of 600 μM as the plasma protein concentration, the unbound fraction in plasma can be calculated using this formula:

$${f}_{u} = \frac{1}{1 + \frac{[ P ]}{{K}_{d , plasma}}} = \frac{1}{1 + \frac{600 uM}{{K}_{d , plasma}}}$$

Experimentally, a final concentration of 250 nM of Compound 1a in PBS-buffer was incubated with various concentrations of immobilised HSA, 0.01% P188 and three different concentrations of plasma (the final dilution of plasma from each species are listed in Table 10).

TABLE 10
The final dilutions of plasma in the incubations from each species.

| Dilution No. | 1 | 2 | 3 |
|---|---|---|---|
| Mouse | 1:5 | 1:15 | 1:45 |
| Rat | 1:5 | 1:15 | 1:45 |
| Rabbit | 1:5 | 1:15 | 1:45 |
| Dog | 1:5 | 1:15 | 1:45 |
| Human | 1:10 | 1:30 | 1:90 |

Two reference solutions were prepared at each plasma dilution. The samples were incubated for two hours at 37° C. under mechanical shaking on a KingFisher robot (Thermo Fisher Scientific) using 1% OVA coated deep-well KingFisher plates and tip combs. After incubation, the samples are centrifuged for 10 min at 750 g to remove the beads. Prior to sample quantification, one volume of supernatant is diluted with one volume of LYD pig plasma to matrix match. The mixtures are assayed for Compound 1a.

The calculation of the pseudo-dissociation constant to plasma proteins is based on 15 data points (five data points with each plasma dilution and three plasma dilutions). For each individual plasma dilution, one of the reference data points and another data point could be excluded from the calculation.

### Sample Analysis by LOCl

All samples were analysed for Compound 1a using Luminescence Oxygen Channelling Immunoassay (LOCI) also called AlphaLISA, which is a homogenous bead-based sandwich immunoassay technology. The measured signal depends on the proximity of two types of beads. Donor beads are coated with streptavidin, while acceptor beads in this case were conjugated with soluble growth hormone binding protein recognizing the hGH receptor binding part of the molecule. The second binding partner in the sandwich is a biotinylated monoclonal antibody (0195-0000-0127) recognizing another epitope of the growth hormone analogue. During the assay, the three reactants combine with analyte to form a bead-aggregate-immune complex. Illumination of the complex with a laser releases singlet oxygen from the donor beads which channels into the acceptor beads and triggers chemiluminescence which is measured. The amount of light generated is proportional to the concentration of the hGH analogue. Standard curves were made in 50%/50% LYD pig plasma/PBS with a resulting lower limit of quantification of 50 μM for the Compound 1a analogue.

The experiments for determining the binding affinity of Compound 1a towards immobilized HSA (K_{d,HSAimm}) was conducted and analysed in parallel with each plasma protein binding experiment. The results from 13 individual experiments passed the criteria are shown in Table 11 and used for calculating the average K_{d,HSAimm}. The average K_{d,HSAimm} value was calculated to be 1.69×10⁻⁶ M, which was used in the calculations of all subsequent plasma protein binding experiments.

TABLE 11
Individual calculated values of Kd, HSAimm (M) of Compound
1a towards immobilised HSA. The mean value is used
for calculating the unbound fraction in plasma.

| Experiment number | K_{d, HSAimm} (M) | R² | Data points excluded |
|---|---|---|---|
| 1 | 1.83E−06 | 0.9930 | 0/6 |
| 2 | 2.03E−06 | 0.9758 | 0/6 |
| 3 | 1.44E−06 | 0.9967 | 0/6 |
| 4 | 2.06E−06 | 0.9806 | 1/6 |
| 5 | 1.54E−06 | 0.9837 | 0/6 |
| 6 | 1.38E−06 | 0.9921 | 1/6 |
| 7 | 1.31E−06 | 0.9864 | 0/6 |
| 8 | 1.55E−06 | 0.9828 | 0/6 |
| 9 | 1.68E−06 | 0.9916 | 1/6 |
| 10 | 1.38E−06 | 0.9799 | 1/6 |
| 11 | 1.76E−06 | 0.9907 | 0/6 |
| 12 | 2.16E−06 | 0.9920 | 0/6 |
| 13 | 1.82E−06 | 0.9874 | 0/6 |
| Mean | 1.69E−06 | — | — |
| SD | 2.82E−07 | — | — |
| % CV | 16.7 | — | — |

The determination of the unbound fraction (fu) in the plasma protein binding experiments in plasma from mice, rats, rabbits, dogs, and humans are listed with individual and average values in Table 12. The average unbound fraction (as a percentage), fu, of Compound 1a was 1.42% in mouse plasma, 0.62% in rat plasma, 0.41% in rabbit plasma, 0.22% in dog plasma and 0.08% in human plasma.

TABLE 12
Individual and average unbound fractions (fu (%)) of Compound
1a in plasma from mice, rats, rabbits, dogs, and humans.

| | | | Rabbits | | |
|---|---|---|---|---|---|
| | | Rat | (New | | |
| Experiment | Mice | (Sprague- | Zealand | Dogs | |
| No. | (C57BL/6) | Dawley) | White) | (Beagle) | Human |
| #1 | 0.97% | 0.56% | 0.31% | 0.17% | 0.083% |
| #2 | 1.46% | 0.52% | 0.41% | 0.24% | 0.079% |
| #3 | 1.82% | 0.78% | 0.51% | 0.25% | 0.079% |
| Mean | 1.42% | 0.62% | 0.41% | 0.22% | 0.08% |
| SD | 0.003470 | 0.001132 | 0.000800 | 0.000317 | 0.000015 |
| % CV | 24.49% | 18.22% | 19.48% | 14.46% | 1.92% |

### Conclusion

The in vitro plasma protein binding of Compound 1a was determined in plasma from mice, rats, rabbits, dogs, and humans. The fractions of unbound Compound 1a in plasma were in the range from 0.08 to 1.42%, with the lowest unbound fraction in plasma in the following order: Humans<dogs<rabbits<rats<mice.

### REFERENCES

1. Kurtzhals, P., Havelund, S., Jonassen, I., Kiehr, B., Larsen, U. D., Ribel, U., and Markussen, J. (1995) Albumin binding of insulins acylated with fatty acids: characterization of the ligand-protein interaction and correlation between binding affinity and timing of the insulin effect in vivo. Biochem. J. 312 (3), 725-731.
2. Kurtzhals, P., Havelund, S., Jonassen, I., Kiehr, B., Ribel, U., and Markussen, J. (1996) Albumin binding and time action of acylated insulins in various species. J. Pharm. Sci. 85 (3), 304-308.

### Example 9: In Vitro Growth Hormone Receptor (GHR)-Mediated Internalisation of Compound 1a, Recombinant Human Growth Hormone (hGH), and Pegvisomant

The aim was to investigate internalisation of Compound 1a, hGH, and Pegvisomant using human GHR expressing baby hamster kidney fibroblasts (BHK21).

A quantitative in vitro study of the internalisation of Compound 1a, hGH, and the GHR antagonist pegvisomant was performed using human GHR-expressing baby hamster kidney fibroblasts (BHK21). Compound 1a, hGH, and Pegvisomant were conjugated with Alexa Fluor 647 dye to follow the internalisation. Labelling was performed in 50 mM Hepes buffer, pH 7.5 using Alexa Flour 647 NHS from Life Technologies. Labelling was performed randomly on the available epsilon-lysine amines. The mono labelled compounds were then isolated using ion-exchange chromatography, and the buffer was exchanged into PBS using Zeba Spin Desalting Columns (Cat. No.: 89893 from Thermo Scientific, 7 kDa cut off). Labelling degree was determined by LCMS (hGH, Compound 1a) or fluorometric (Pegvisomant). The labelling degree was 1.0, 1.0 and 0.7 for hGH, Compound 1a and Pegvisomant respectively.

### Cell Line and Culture Conditions

Baby hamster kidney cells (BHK21) overexpressing the human growth hormone receptor (BHK21/hGHR), previously employed in in-house functional compound screening activities, were utilized for the internalization assay. The hGHR overexpression is essential to achieve higher levels of internalization. BHK21/hGH cells were generated using a pcDNA3, 1+(neo) plasmid containing hGHR sequence. Additionally, a Stat3-Luciferase (puromycin resistance) encoding plasmid was transfected into the cells for functional screening purposes that is not of relevance for the assay described here. Transfected cells were selected by using 600 μg/ml G418 (Gibco #10131-027) and 1 μg/ml puromycin (Gibco #11138-03) to generate a stable pool that was further used for monoclonal cell line selection.

For maintenance, cells were cultured in DMEM supplemented with Glutamax (Gibco #31966-021), 10% FBS FBS (Thermo Fisher #10091148), and 1% Penicillin-Streptomycin (Gibco #15140-122), 600 μg/ml G418 and 1 μg/ml puromycin under 37° C. and 5% CO₂ conditions. For the assay, cells were seeded in culture media with reduced FBS content of 1% and no selection markers G418 and puromycin.

Internalization 20.000 cells were seeded in assay media in 96-well PhenoPlatae™ (Revvity #6055302). For time-course experiments, one plate for each time point was prepared: 10, 20, 30, 60, 90, 120-, 180-, 240- and 1440-min. Cells were incubated over night at 37° C. and 5% CO₂. Next day, cells were treated with compounds and internalization was observed using the Operetta CLS High-Content Analysis System (Revvity). Compounds were titrated in cell seeding media supplemented with 1% BSA (Miltenyi Biotech #130-091-476) instead of 1% FBS. Titrations were ranging 10-0,0045 nM. Cell plates were incubated according to indicated time points, washed once briefly with 4% PFA in PBS (Ampliqon #AMPQ43226) and then fixated with 4% PFA in PBS for 20 min. Cells were washed three times with PBS and stored at 4° C. until staining. For nucleus staining, 1:10.000 Hoechst 33342 (Invitrogen #H3570) in PBS was applied. After 15 min of treatment, cells were washed with PBS (Gibco #14040-091). Cells were captured by confocal imaging.

### Confocal Imaging

Internalization was assessed using the Operetta Software Harmony. Cells were captured with a 40× water objective and Hoechst 33342 and Alexa Fluor 647 channels. Nine fields per well were imaged and analysed. The analysis sequence was as follows: Find nuclei by Hoechst 33342, find cytoplasm using the nuclei to identify cells, select population by adjusting the cytoplasm area and nucleus intensity and area, find spots and select population by adjusting spot intensity and area. This analysis sequence led to the result of “relative spot intensity-mean per well”. Further analysis was done in GraphPad Prism 9.0.1. To characterize the internalization of the compounds, a non-linear regression one-phase association fitting was applied on the obtained data. While compounds have been tested at different concentrations, only data from 1 nM compound concentration was used to determine half-life and maximum.

Cells were fixated at predetermined timepoints from 0-4 hours and fluorescence determined for each combination of time point and concentration level.

The results showed that all three compounds were internalised by the GHR in the concentration range 0.0045-10 nM. The internalisation rate was highest for hGH followed by Compound 1a and pegvisomant with a similar but lower internalisation rate. Internalisation was reduced in the presence of a nonlabelled competitor.

Addition of non-labelled Compound 1a and Pegvisomant to cells with labelled hGH showed a displacement effect of both Compound 1a and Pegvisomant on the GHR suggesting that uptake is specific to the receptor.

The displacement effect for hGH was higher at 4 hours compared to 24 hours after addition of Compound 1a and Pegvisomant compared to 24 hours, the displacement effect for somapacitan lasted for 24 hours.

### Conclusion

Compound 1a undergoes receptor-mediated internalisation by the GHR at a slower rate than human GH, but faster than pegvisomant. Compound 1a is more easily displaced from the GHR by pegvisomant compared to human GH.

### Example 10: Synthesis of Additional Albumin Binders

In similar way, the bromo acetamide derived albumin binders below were made using solid phase method as described in U.S. Pat. No. 8,779,109 B2, U.S. Pat. No. 9,895,417 B2 or similar to Example 1. The bromo acetamides may be used directly for coupling to the reduced free cysteine residue on the protein (as described in U.S. Pat. No. 8,779,109 B2 example 44 or in U.S. Pat. No. 9,895,417 B2 example 5.1). Alternatively they may be converted to the iodo acetates as described in Example 1.ii and used for coupling to the free cysteine residue on the protein as in Example 1.iii.

Br-Chem. 6a: (8S, 31S)-1-Bromo-2, 10, 19,28,33-pentaoxo-12, 15,21,24-tetraoxa-3,9, 18,27,32-pentaazanonatetracontane-8,31,49-tricarboxylic acid

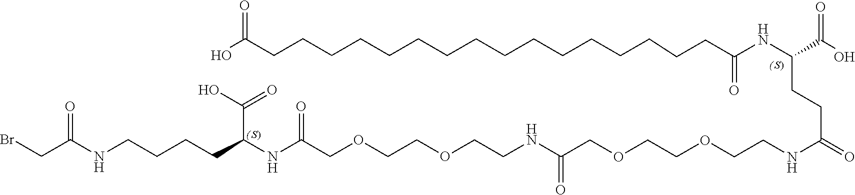

This compound was prepared on solid support using standard Fmoc-peptide chemistry on an ABI433 synthesizer. Peptide was assembled on Fmoc-Lys (Mtt)-Wang resin using Fmoc-Ado-OH, Fmoc-Glu-OtBu and octadecanedioic acid mono-tert-butyl ester. Couplings were performed using 5-chloro-1-((dimethylamino) (dimethyliminio)methyl)-1H-benzo[d][1,2,3]triazole 3-oxide tetrafluoroborate (TCTU, 1 equivalent) and DIPEA (2 equivalents) in DMF. The Mtt group was removed by treatment with 80% 1,1,1,3,3,3-hexafluoropropan-2-ol (HPIF) in dicloromethane, and the final coupling of bromoacetic acid was performed with N,N′-diisopropylcarbodiimide (DIC). Compound was cleaved from resin using trifluoroacetic acid in DCM (2:1). Resin was filtered off and washed with dichloromethane. The solvent was evaporated and acetonitrile was added to the residue. White precipitate was filtered, washed with acetonitrile and diethyl ether and dried in vacuo to yield the title compound as white powder. 1H NMR spectrum (300 MHz, AcOD-d4, dH): 4.68 (dd, J=8.3 and 5.0 Hz, 1H); 4.61 (dd, J=8.9 and 4.7 Hz, 1H); 4.17 (s, 2H); 4.12 (s, 2H); 3.96 (s, 2H); 3.78-3.60 (m, 12H); 3.58-3.40 (m, 4H); 3.31 (t, J=6.8 Hz, 2H); 2.45 (t, J=7.3 Hz, 2H); 2.39-2.30 (m, 4H); 2.29-2.17 (m, 1H); 2.16-2.06 (m, 1H); 2.02-1.92 (m, 1H); 1.90-1.75 (m, 1H); 1.69-1.55 (m, 6H); 1.52-1.41 (m, 2H); 1.30 (s, 24H). LC-MS: Rt=2.82 min. LC-MS m/z: 984.5 (M+H)+.

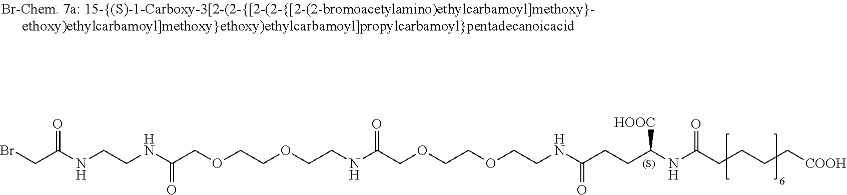

This compound was prepared as described in U.S. Pat. No. 9,895,417 B2 example 4.1.

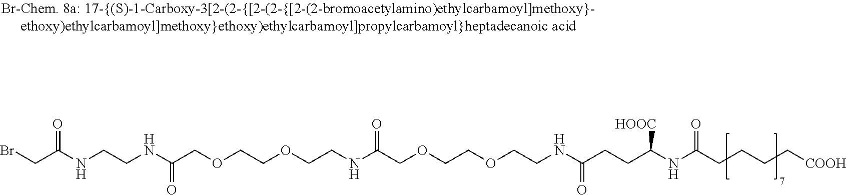

This compound was prepared as described in U.S. Pat. No. 9,895,417 B2 example 4.5.

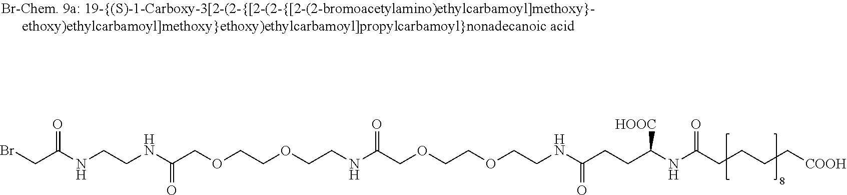

This compound was prepared as described in U.S. Pat. No. 9,895,417 B2 example 4.4.

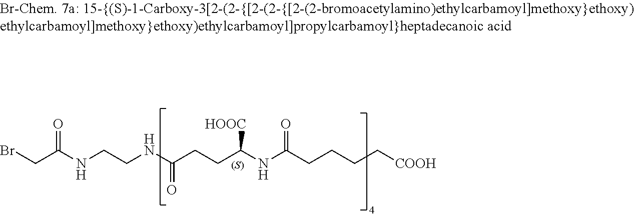

This compound was prepared on solid support using standard Fmoc-peptide chemistry on an ABI433 synthesizer. The peptide was assembled on 2-chlorotrityl chloride resin using Fmoc-Glu-OtBu and octadecanedioic acid mono-tert-butyl ester, generally following the protocol U.S. Pat. No. 9,895,417 B2 example 4.1. ¹H NMR spectrum (300 MHz, AcOD-d4, dH): 4.69-4.57 (m, 4H); 3.95 (s, 2H); 3.50-3.31 (m, 4H); 2.57-2.21 (m, 16H); 1.71-1.55 (m, 4H); 1.30 (bs, 24H). LC-MS Rt=2.45 min. LC-MS m/z: 994.0 (M+H)+.

### Example 11: Synthesis and Characterization of Additional GH Receptor Antagonists (GHRA's)

Compounds 3-19 were prepared using essentially the procedure outlined in Example 1, by the following general steps:
i) GHRA peptide backbone expression in E. coli to obtain the Cys101 cystamine protected mixed disulphide.
ii) Synthesis of the iodoacetamide of the albumin binding moiety.
iii) Deprotection of the Cys101 mixed disulphide and nucleophilic substitution with the iodoacetamide.

Details of each step are shown in Example 1 using the appropriate chemical which will be apparent based on the structure of the target GHRA, alternatively, the compounds can be obtained using the procedures essentially as described in WO2011/089255, (see page 132, line 17-page 137; Example 44, page 161, line 5-page 164, line 11). Characterization data is provided in Table 13.

TABLE 13
Characterization Data for hGH antagonist conjugates (compounds)

| | Growth | | | | | Retention | |
|---|---|---|---|---|---|---|---|
| LA- | hormone | Albumin | | Calculated | Found | time | Purity |
| GHRA | variant | binder | Sum Formula | Mass | LCMS* | (UPLC) | % |
| Compound | GHv-1 | Chem. 1a | C1028 H1596 | 23206 Da | 23206 | 4.463 min | >99 |
| 1a | | | N272 O322 S9 | | Da | | |
| Compound | GHv-2 | Chem. 1a | C1028 H1594 | 23208 Da | 23206 | 4.541 min | >99 |
| 3a | | | N270 O324 S9 | | Da | | |
| Compound | GHv-1 | Chem. 6a | C1020 H1584 | 22918 Da | 22916 | 4.631 min | >99 |
| 4a | | | N266 O318 S8 | | Da | | |
| Compound | GHv-1 | Chem. 7a | C1014 H1574 | 22803 Da | 22802 | 4.554 min | 98.6 |
| 5a | | | N266 O316 S8 | | Da | | |
| Compound | GHv-1 | Chem. 8a | C1016 H1578 | 22831 Da | 22829 | 4.631 min | >99 |
| 6a | | | N266 O316 S8 | | Da | | |
| Compound | GHv-1 | Chem. 9a | C1018 H1582 | 22860 Da | 22858 | 4.723 min | >99 |
| 7a | | | N266 O316 S8 | | Da | | |
| Compound | GHv-1 | Chem. 10a | C1019 H1577 | 22929 Da | 22928 | 4.618 min | >99 |
| 8a | | | N267 O319 S8 | | Da | | |
| Compound | GHv-2 | Chem. 6a | C1020 H1582 | 22920 Da | 22919 | 4.635 min | >99 |
| 9a | | | N264 O320 S8 | | Da | | |
| Compound | GHv-2 | Chem. 7a | C1014 H1572 | 22805 Da | 22804 | 4.562 min | >99 |
| 10a | | | N264 O318 S8 | | Da | | |
| Compound | GHv-2 | Chem. 8a | C1016 H1576 | 22834 Da | 22832 | 4.640 min | >99 |
| 11a | | | N264 O318 S8 | | Da | | |
| Compound | GHv-2 | Chem. 9a | C1018 H1580 | 22862 Da | 22860 | 4.735 min | >99 |
| 12a | | | N264 O318 S8 | | Da | | |
| Compound | GHv-2 | Chem. 10a | C1019 H1575 | 22930 Da | 22929 | 4.596 min | >99 |
| 13a | | | N265 O321 S8 | | Da | | |
| Compound | GHv-3 | Chem. 1a | C1031 H1601 | 23237 Da | 23237 | 4.494 min | 98.28 |
| 14a | | | N269 O324 S9 | | Da | | |
| Compound | GHv-3 | Chem. 6a | C1023 H1589 | 22949 Da | 22948 | 4.567 min | >99 |
| 15a | | | N263 O320 S8 | | Da | | |
| Compound | GHv-3 | Chem. 7a | C1017 H1579 | 22835 Da | 22834 | 4.499 min | >99 |
| 16a | | | N263 O318 S8 | | Da | | |
| Compound | GHv-3 | Chem. 8a | C1019 H1583 | 22863 Da | 22861 | 4.637 min | >99 |
| 17a | | | N263 O318 S8 | | Da | | |
| Compound | GHv-3 | Chem. 9a | C1021 H1587 | 22891 Da | 22889 | 4.717 min | >99 |
| 18a | | | N263 O318 S8 | | Da | | |
| Compound | GHv-3 | Chem. 10a | C1022 H1582 | 22960 Da | 22958 | 4.582 min | >99 |
| 19a | | | N264 O321 S8 | | Da | | |

*Mass spectra were recorded in the 200-3000 Da range and deconvoluted using MassLynx version 4.2.

### Example 12: GH Receptor Affinity by Surface Plasmon Resonance (SPR) for Additional GHRA's

The purpose of the Example was to determine the affinity of the compound of the present invention in comparison with pegvisomant. The SPR experiments were performed using a Biacore T200 and Biacore 8K instruments (Cytiva) at 25° C. Binding kinetics were determined using a Avi-tag biotinylated version of the extracellular domain of the growth hormone receptor (hGHR-ECD) comprising hGHR residues 19-264 and a Fc conjugated dimeric hGHR-ECD-Fc construct.

For the biotinylated hGHR-ECD, the Biotin CAPture kit (Cytiva; 28920233) was used which enables reversible capture of biotinylated ligands. The chip surface was prepared by rehydration in HBS-EP buffer (0.01 M HEPES pH 7.4, 0.15 M NaCl, 3 mM EDTA, 0.005% v/v Surfactant P20) in the instrument using stand-by flow. Prior to the experiment the chip surface was conditioned using Regeneration solution (3 parts of Regeneration Stock 1 (8 M guanidine-HCl) and 1 part Regeneration Stock 2 (1 M NaOH)) followed by three consecutive start-up cycles using HBS-EP buffer as analyte. Each cycle consisted of the following steps: 1) CAPture reagent (included in Biotion CAPture kit) 50 μg/mL in HBS-EP buffer (Flow rate: 2 μl/min, Contact time: 300 sec); 2) Biotinylated ligand capture (Flow rate: 10 μl/min, Contact time: 180 sec); 3) Analyte (Compound 1a, Compound 2, Somatropin and Pegvisomant) injection (Flow rate: 30 μl/min, Contact time: 240 sec, Dissociation time: 1200 sec); and 4) Regeneration using Regeneration solution (Flow rate: 10 μl/min, Contact time 60 sec). The analytes were all diluted in HBS-EP buffer and applied to the ligand coated chip in concentrations of 100, 20, 4, 0.8, 0.16, 0.032 and 0 nM.

For the dimeric hGHR-ECD-Fc we used a Sensor Chip Protein A (Cytiva; 29127555). The chip surface was prepared by rehydration in HBS-EP buffer in the instrument using stand-by flow. Prior to the experiment the chip surface was conditioned using Regeneration solution using Regeneration solution 2 (10 mM Glycine-HCl pH 1.5, Cytiva; BR100354) followed by three consecutive start-up cycles using buffer as analyte. Each cycle consisted of the following steps: 1) Ligand capture (Flow rate: 10 μl/min, Contact time: 30 seconds). 2) Analyte injection (Flow rate: 50 μl/min, Contact time: 240 seconds, Dissociation time: 1200 seconds) and 3) Regeneration using Regeneration solution 2 (Flow rate 30 μl/min Contact time 30 sec). The analytes were all diluted in HBS-EP buffer and applied to the ligand coated chip in concentrations of 100, 20,4, 0.8,0.16, 0,032 and 0 nM.

All dilutions and experiments were repeated in HBS-EP buffer containing 1% Bovine Serum Albumin (BSA, Sigma-Aldrich: A4503)

The data analysis was performed using the Biacore 8K evaluation software. Kinetic fitting was performed after baseline subtraction and curves were globally fitted using a 1:1 Langmuir binding model. K_{D} values were calculated based on the fitted association (kₐ) and dissociation constants (k_{d}). Results are listed in Table 14.

TABLE 14
Kinetic values determined by SPR for
binding to monomeric hGHR-ECD.

| | ka | | kd | | KD | |
|---|---|---|---|---|---|---|
| | (1/Ms) | | (1/s) | | (nM) | |
| | Mean | SD | Mean | SD | Mean | SD |
| GHv-1 | 1.6E+05 | 2.8E+03 | 3.0E−05 | 1.2E−05 | 0.19 | 0.07 |
| Compound 1a | 2.3E+05 | 7.9E+03 | 2.7E−05 | 1.2E−06 | 0.12 | 0.01 |
| Compound 3a | 3.0E+05 | 1.1E+04 | 3.1E−05 | 2.9E−06 | 0.11 | 0.01 |
| Compound 4a | 2.7E+05 | 9.0E+03 | 2.9E−05 | 6.5E−07 | 0.11 | 0.00 |
| Compound 5a | 2.6E+05 | 3.0E+03 | 2.6E−05 | 1.5E−06 | 0.10 | 0.01 |
| Compound 6a | 2.8E+05 | 5.6E+03 | 2.9E−05 | 5.0E−06 | 0.10 | 0.02 |
| Compound 7a | 3.0E+05 | 1.2E+04 | 2.6E−05 | 3.7E−06 | 0.09 | 0.01 |
| Compound 8a | 1.9E+05 | 6.7E+03 | 2.6E−05 | 3.8E−06 | 0.13 | 0.02 |
| Compound 9a | 4.3E+05 | 1.4E+04 | 2.6E−05 | 1.1E−06 | 0.06 | 0.00 |
| Compound 10a | 2.6E+05 | 9.7E+03 | 2.6E−05 | 1.7E−06 | 0.10 | 0.00 |
| Compound 11a | 2.8E+05 | 8.0E+03 | 2.4E−05 | 1.7E−06 | 0.09 | 0.01 |
| Compound 12a | 4.2E+05 | 2.2E+04 | 2.7E−05 | 1.7E−06 | 0.06 | 0.00 |
| Compound 13a | 3.5E+05 | 1.5E+04 | 2.7E−05 | 4.9E−07 | 0.08 | 0.00 |
| Compound 14a | 3.3E+05 | 5.6E+03 | 2.8E−05 | 8.9E−07 | 0.08 | 0.00 |
| Compound 15a | 3.2E+05 | 1.4E+04 | 2.8E−05 | 1.4E−06 | 0.09 | 0.01 |
| Compound 16a | 3.3E+05 | 1.3E+04 | 2.7E−05 | 2.1E−06 | 0.08 | 0.00 |
| Compound 17a | 3.2E+05 | 5.0E+03 | 2.5E−05 | 2.4E−08 | 0.08 | 0.00 |
| Compound 18a | 3.2E+05 | 3.8E+03 | 2.7E−05 | 2.4E−06 | 0.09 | 0.01 |
| Compound 19a | 2.8E+05 | 3.1E+03 | 2.7E−05 | 3.6E−07 | 0.10 | 0.00 |
| Norditropin | 1.1E+06 | 6.0E+04 | 1.2E−04 | 2.0E−06 | 0.11 | 0.01 |
| Pegvisomant | 1.2E+04 | 2.5E+03 | 3.2E−05 | 8.2E−07 | 2.67 | 0.49 |
| Somapacitan | 4.8E+05 | 2.5E+04 | 1.6E−04 | 1.8E−06 | 0.34 | 0.02 |

SD: Standard Deviation (n = 3). Standard

TABLE 15
Kinetic values determined by SPR for binding to monomeric
hGHR-ECD in presence of 1% BSA.

| | ka | | kd | | KD | |
|---|---|---|---|---|---|---|
| | (1/Ms) | | (1/s) | | (nM) | |
| | Mean | SD | Mean | SD | Mean | SD |
| GHv-1 | 1.8E+05 | 1.1E+04 | 2.3E−05 | 1.3E−06 | 0.13 | 0.00 |
| Compound 1a | 4.1E+05 | 2.8E+04 | 2.6E−05 | 6.1E−06 | 0.06 | 0.01 |
| Compound 3a | 5.0E+05 | 1.3E+05 | 3.3E−05 | 6.7E−06 | 0.07 | 0.01 |
| Compound 4a | 2.8E+05 | 5.7E+04 | 7.0E−05 | 3.1E−06 | 0.25 | 0.04 |
| Compound 5a | 2.4E+05 | 2.5E+03 | 3.8E−05 | 2.5E−06 | 0.16 | 0.01 |
| Compound 6a | 2.6E+05 | 8.5E+04 | 7.1E−05 | 5.0E−06 | 0.29 | 0.09 |
| Compound 7a | 3.0E+05 | 2.0E+05 | 3.6E−05 | 1.5E−05 | 0.24 | 0.30 |
| Compound 8a | 3.9E+05 | 2.7E+05 | 4.5E−05 | 1.8E−05 | 0.27 | 0.36 |
| Compound 9a | 2.3E+05 | 1.0E+04 | 8.6E−05 | 7.0E−06 | 0.37 | 0.02 |
| Compound 10a | 2.5E+05 | 1.3E+04 | 3.9E−05 | 3.5E−06 | 0.16 | 0.01 |
| Compound 11a | 2.7E+05 | 1.1E+05 | 8.1E−05 | 6.2E−06 | 0.34 | 0.16 |
| Compound 12a | 3.2E+05 | 1.2E+05 | 4.7E−05 | 5.7E−06 | 0.15 | 0.03 |
| Compound 13a | 1.9E+05 | 1.6E+04 | 7.1E−05 | 6.8E−06 | 0.38 | 0.02 |
| Compound 14a | 3.3E+05 | 1.9E+04 | 3.6E−05 | 4.4E−06 | 0.11 | 0.01 |
| Compound 15a | 3.0E+05 | 6.4E+04 | 8.4E−05 | 6.7E−06 | 0.29 | 0.05 |
| Compound 16a | 3.1E+05 | 8.9E+03 | 4.2E−05 | 1.1E−06 | 0.13 | 0.01 |
| Compound 17a | 3.6E+05 | 3.8E+04 | 7.9E−05 | 9.7E−06 | 0.22 | 0.00 |
| Compound 18a | 3.1E+05 | 2.2E+05 | 3.7E−05 | 2.0E−05 | 0.28 | 0.38 |
| Compound 19a | 3.1E+05 | 1.4E+05 | 7.5E−05 | 1.1E−05 | 0.31 | 0.22 |
| Norditropin | 1.1E+06 | 1.5E+04 | 1.2E−04 | 1.4E−06 | 0.11 | 0.00 |
| Pegvisomant | 9.7E+03 | 1.5E+03 | 3.9E−05 | 4.7E−06 | 4.12 | 1.13 |
| Somapacitan | 1.6E+05 | 7.1E+03 | 2.3E−04 | 8.0E−06 | 1.45 | 0.04 |

SD: Standard Deviation (n =3 ).

TABLE 16
Kinetic values determined by SPR for binding to dimeric
hGHR-ECD-Fc.

| | ka | | kd | | KD | |
|---|---|---|---|---|---|---|
| | (1/Ms) | | (1/s) | | (nM) | |
| | Mean | SD | Mean | SD | Mean | SD |
| GHv-1 | 2.1E+05 | 6.8E+03 | 5.0E−05 | 2.6E−05 | 0.24 | 0.13 |
| Compound 1a | 2.9E+05 | 9.8E+03 | 4.9E−05 | 1.0E−05 | 0.17 | 0.04 |
| Compound 3a | 3.6E+05 | 1.2E+04 | 5.3E−05 | 8.8E−06 | 0.15 | 0.03 |
| Compound 4a | 3.1E+05 | 8.7E+03 | 4.4E−05 | 8.4E−06 | 0.14 | 0.03 |
| Compound 5a | 3.2E+05 | 5.6E+03 | 4.4E−05 | 4.4E−06 | 0.14 | 0.02 |
| Compound 6a | 3.2E+05 | 7.7E+03 | 4.5E−05 | 9.5E−06 | 0.14 | 0.03 |
| Compound 7a | 3.4E+05 | 8.1E+03 | 4.2E−05 | 6.7E−06 | 0.12 | 0.02 |
| Compound 8a | 2.4E+05 | 3.6E+03 | 4.3E−05 | 1.5E−06 | 0.18 | 0.01 |
| Compound 9a | 4.8E+05 | 5.8E+03 | 4.7E−05 | 4.9E−06 | 0.10 | 0.01 |
| Compound 10a | 3.1E+05 | 7.5E+03 | 3.9E−05 | 7.1E−06 | 0.12 | 0.02 |
| Compound 11a | 3.3E+05 | 5.5E+03 | 4.0E−05 | 4.9E−06 | 0.12 | 0.01 |
| Compound 12a | 4.7E+05 | 2.1E+04 | 4.2E−05 | 1.2E−06 | 0.09 | 0.01 |
| Compound 13a | 3.8E+05 | 6.1E+03 | 4.7E−05 | 4.0E−06 | 0.12 | 0.01 |
| Compound 14a | 3.7E+05 | 2.7E+04 | 4.8E−05 | 6.5E−06 | 0.13 | 0.03 |
| Compound 15a | 3.4E+05 | 7.6E+03 | 4.6E−05 | 3.0E−06 | 0.13 | 0.01 |
| Compound 16a | 3.6E+05 | 5.3E+03 | 4.4E−05 | 1.1E−06 | 0.12 | 0.00 |
| Compound 17a | 3.4E+05 | 2.6E+03 | 4.1E−05 | 4.4E−06 | 0.12 | 0.01 |
| Compound 18a | 3.6E+05 | 1.1E+04 | 4.4E−05 | 3.6E−06 | 0.12 | 0.01 |
| Compound 19a | 3.3E+05 | 4.3E+03 | 4.7E−05 | 2.1E−06 | 0.14 | 0.01 |
| Norditropin | 3.0E+06 | 8.8E+04 | 4.0E−05 | 2.3E−06 | 0.01 | 0.00 |
| Pegvisomant | 1.5E+04 | 4.4E+03 | 1.1E−04 | 4.5E−06 | 7.69 | 1.76 |
| Somapacitan | 1.3E+06 | 1.1E+05 | 4.4E−05 | 2.1E−07 | 0.03 | 0.00 |

SD: Standard Deviation (n = 3).

TABLE 17
Kinetic values determined by SPR for binding to dimeric
hGHR-ECD-Fc inpresence of 1% BSA.

| | ka | | kd | | KD | |
|---|---|---|---|---|---|---|
| | (1/Ms) | | (1/s) | | (nM) | |
| | Mean | SD | Mean | SD | Mean | SD |
| GHv-1 | 2.3E+05 | 7.3E+03 | 3.5E−05 | 2.6E−05 | 0.16 | 0.12 |
| Compound 1a | 1.2E+05 | 2.8E+04 | 3.4E−05 | 2.8E−06 | 0.30 | 0.09 |
| Compound 3a | 1.2E+05 | 3.4E+03 | 3.5E−05 | 3.0E−06 | 0.31 | 0.02 |
| Compound 4a | 1.4E+05 | 3.3E+03 | 3.5E−05 | 6.9E−06 | 0.25 | 0.05 |
| Compound 5a | 2.7E+05 | 1.2E+04 | 4.5E−05 | 8.2E−06 | 0.17 | 0.02 |
| Compound 6a | 1.4E+05 | 3.3E+03 | 3.7E−05 | 5.3E−06 | 0.26 | 0.03 |
| Compound 7a | 1.1E+05 | 1.0E+04 | 2.7E−05 | 3.3E−06 | 0.25 | 0.01 |
| Compound 8a | 1.1E+05 | 9.1E+03 | 4.2E−05 | 4.1E−06 | 0.38 | 0.03 |
| Compound 9a | 1.9E+05 | 2.3E+03 | 2.8E−05 | 5.4E−06 | 0.15 | 0.03 |
| Compound 10a | 2.7E+05 | 8.2E+03 | 3.6E−05 | 1.3E−05 | 0.13 | 0.05 |
| Compound 11a | 1.4E+05 | 1.8E+03 | 2.9E−05 | 3.3E−06 | 0.21 | 0.02 |
| Compound 12a | 1.4E+05 | 3.3E+03 | 2.2E−05 | 6.4E−06 | 0.16 | 0.04 |
| Compound 13a | 1.6E+05 | 4.4E+03 | 3.3E−05 | 7.1E−06 | 0.20 | 0.04 |
| Compound 14a | 1.4E+05 | 1.7E+04 | 3.2E−05 | 4.8E−06 | 0.23 | 0.01 |
| Compound 15a | 1.4E+05 | 1.1E+03 | 3.5E−05 | 7.8E−06 | 0.24 | 0.05 |
| Compound 16a | 3.2E+05 | 1.5E+04 | 4.6E−05 | 4.8E−06 | 0.14 | 0.01 |
| Compound 17a | 1.4E+05 | 4.3E+03 | 2.7E−05 | 4.3E−06 | 0.19 | 0.04 |
| Compound 18a | 1.1E+05 | 2.5E+03 | 3.1E−05 | 6.0E−06 | 0.29 | 0.05 |
| Compound 19a | 1.3E+05 | 3.2E+03 | 3.7E−05 | 5.6E−06 | 0.28 | 0.04 |
| Norditropin | 3.3E+06 | 2.8E+05 | 3.9E−05 | 3.1E−06 | 0.01 | 0.00 |
| Pegvisomant | 1.9E+04 | 1.8E+03 | 1.1E−04 | 4.4E−06 | 5.78 | 0.69 |
| Somapacitan | 4.2E+05 | 1.4E+04 | 4.6E−05 | 6.0E−06 | 0.11 | 0.01 |

SD: Standard Deviation (n = 3).

As seen in table 14-17, both Compound 1a and Compound 2 have higher affinity to hGHR-ECD compared to pegvisomant. The prolongation of the pegvisomant molecule is based on pegylation. Pegylation has a negative impact on the receptor binding affinity to the growth hormone receptor with both a slower association and dissociation rate.

### Example 13: Steady State GHR Binding Affinity by Isothermal Titration Calorimetry (ITC) for Additional GHRA's

The purpose of the example was to measure the thermodynamic interactions between the binding partners (receptor/ligand) in solution both with and without HSA present to determine affinities.

Compound 1a, pegvisomant and Fatty acid free human serum albumin (HSA) (Sigma) were dialysed using Slide-A-Lyzer™ dialysis cassettes overnight into 4.38 mM Histidine, 2.05 mM NaCl pH 7.1 buffer. All proteins were subjected to size exclusion chromatography with multi-angle light scattering (SEC-MALS) analyses to determine the concentration before and after the up-concentration and after ITC experiments. SEC-MALS was carried out on an Agilent 1200 Series HPLC system (Agilent, Darmstadt, Germany) using a TSK G3000 SWXL column (Tosoh Bioscience, Tokyo). The column was maintained at a temperature of 20° C. and equilibrated in 122 mM Na₂HPO₄, 78 mM NaH₂PO₄, 300 mM NaCl, 4% 2-propanol, pH 6.8). Detection was done by UV absorption at 280 nm, refractive index (RI) and multi-angle laser light scattering (MALS). RI and MALS detectors were Optilab T-rEX (Wyatt) and MiniDawn Treos (LS) (Wyatt Technology, Santa Barbara, CA, USA), respectively. Data were evaluated using Chemstation B.03.01 and Astra Ver. 6.1.1.17

ITC experiments performed using a PEAQ-ITC calorimeter (Malvern, UK) at 37° C. The sample cell (300 μL) contained 10 UM Compound 1a or Pegvisomant in the presence of HSA (0, 1, 5, or 10 mg/mL). The syringe contained hGHR-ECD (ligand), at 100 μM. A thermal equilibration step was followed by a 60 sec delay and subsequently an initial 0.4-μL injection of hGHR-ECD, followed by 25 injections of 1.5 μL and a final injection of 1.1 μL at an interval of 120 sec. The stirring speed was 750 rpm, and the reference power was kept constant at 9.64 μcal/s. Data treatment done using the PEAQ-ITC analysis software (Malvern Instruments) using a one site fitting model with a fitted offset compensating. Results are shown in Table 18.

TABLE 18
Equilibrium binding parameters at increasing concentrations of HSA using ITC.

| | | | | | ΔH | | ΔG | | TΔS | |
|---|---|---|---|---|---|---|---|---|---|---|
| [HSA] | N (sites) | | K_{D} (nM) | | (kcal/mol) | | (kcal/mol) | | (kcal/mol) | |
| mg/ml | Mean | SEM | Mean | SEM | Mean | SEM | Mean | SEM | Mean | SEM |

Compound 1a

| | | | | | | | | | | |
|---|---|---|---|---|---|---|---|---|---|---|
| 0 | 1.09 | 0.08 | 2.1 | 0.44 | −32.9 | 1.91 | −12.3 | 0.12 | 20.6 | 1.99 |
| 1 | 1.10 | 0.09 | 4.8 | 0.29 | −30.3 | 1.16 | −11.8 | 0.06 | 18.5 | 1.15 |
| 5 | 1.11 | 0.09 | 4.9 | 1.26 | −29.6 | 1.10 | −11.8 | 0.15 | 17.7 | 1.00 |
| 10 | 1.11 | 0.11 | 3.5 | 0.32 | −29.4 | 1.53 | −12.0 | 0.10 | 17.4 | 1.51 |

Pegvisomant

| | | | | | | | | | | |
|---|---|---|---|---|---|---|---|---|---|---|
| 0 | 1.23 | 0.26 | 6.6 | 1.58 | −31.2 | 1.15 | −11.6 | 0.15 | 19.6 | 1.31 |
| 1 | 1.17 | 0.15 | 6.6 | 2.33 | −31.7 | 1.73 | −11.6 | 0.25 | 20.1 | 1.96 |
| 5 | 1.24 | 0.21 | 5.7 | 1.00 | −32.3 | 0.92 | −11.7 | 0.10 | 20.6 | 1.01 |
| 10 | 1.21 | 0.14 | 4.8 | 2.11 | −-31.5 | 1.47 | −11.9 | 0.31 | 19.7 | 1.75 |

SD: Standard Deviation (n = 3)

N is binding stoichiometry, K_{D} is dissociation constant, ΔH is change in enthalpy, ΔG is change in Gibb's free energy, ΔS is change in entropy, and T is temperature. It is seen that K_{D} for compound 1a is lower than pegvisomant, thus, compound 1a also shows higher affinity in the ITC assay. It is also seen that for both compound 1a and pegvisomant, the affinity does not change significant in the presence of HSA as compared to 0 mg/mL HSA.

### Example 14: Studying GHRA's in a STAT3 Assay

A series of hGH antagonists were tested in the antagonistic mode (N=1) with and without human albumin in the media. Data are summarized in table 19 below.

TABLE 19
STAT3 - Assay Data

| | Media | | |
|---|---|---|---|
| | DMEM w/o phenol red | | |
| | (Gibco, 31053-028) + | DMEM w/o phenol red | |
| | 2.5% Human serum | (Gibco, 31053-028) + | |
| | albumin (Sigma, A9511) + | 1% Ovalbumin (Sigma, | |
| | 0.01% Tween-20. | A5503) + 0.01% Tween-20 | Albumin |
| Compound | IC₅₀ (nM) | IC₅₀ (nM) | binder shift |
| GHv-1 | 5.5 | 2.4 | 2.3 |
| Compound 1a | 29.2 | 2.0 | 14.3 |
| Compound 3a | 32.2 | 1.7 | 18.7 |
| Compound 4a | 25.8 | 1.4 | 18.9 |
| Compound 5a | 12.9 | 1.6 | 8.0 |
| Compound 6a | 33.2 | 2.1 | 15.5 |
| Compound 7a | 18.0 | 1.1 | 16.2 |
| Compound 8a | 21.0 | 1.5 | 14.4 |
| Compound 9a | 26.6 | 1.7 | 16.0 |
| Compound 10a | 15.3 | 1.9 | 8.1 |
| Compound 11a | 52.7 | 2.9 | 18.3 |
| Compound 12a | 45.1 | 2.4 | 18.5 |
| Compound 13a | 21.4 | 1.6 | 13.3 |
| Compound 14a | 30.1 | 2.6 | 11.5 |
| Compound 15a | 26.7 | 1.0 | 25.5 |
| Compound 16a | 11.3 | 2.0 | 5.6 |
| Compound 17a | 24.0 | 1.5 | 16.5 |
| Compound 18a | 55.3 | 2.8 | 19.7 |
| Compound 19a | 53.1 | 3.4 | 15.8 |

### Conclusion

The present example demonstrates that the compounds of the present disclosure, in particular compounds 1a, 3a-19a display promising GHRA properties for use in a medical setting.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A long-acting growth hormone receptor antagonist comprising:
a growth hormone variant comprising the following amino acid substitutions as compared to human growth hormone (hGH) (SEQ ID NO: 1):
H18D, H21N, L101C, G120R, R167N, K168A, D171S, K172R, E174S, and I179T;
H18D, H21N, L101C, G120R, N149D, N152D, R167N, K168A, D171S, K172R, E174S, and I179T; or
H18D, H21N, L101C, G120R, N149D, N152D, R167N, D171S, E174S, and I179T; and
an albumin binding moiety selected from the group consisting of 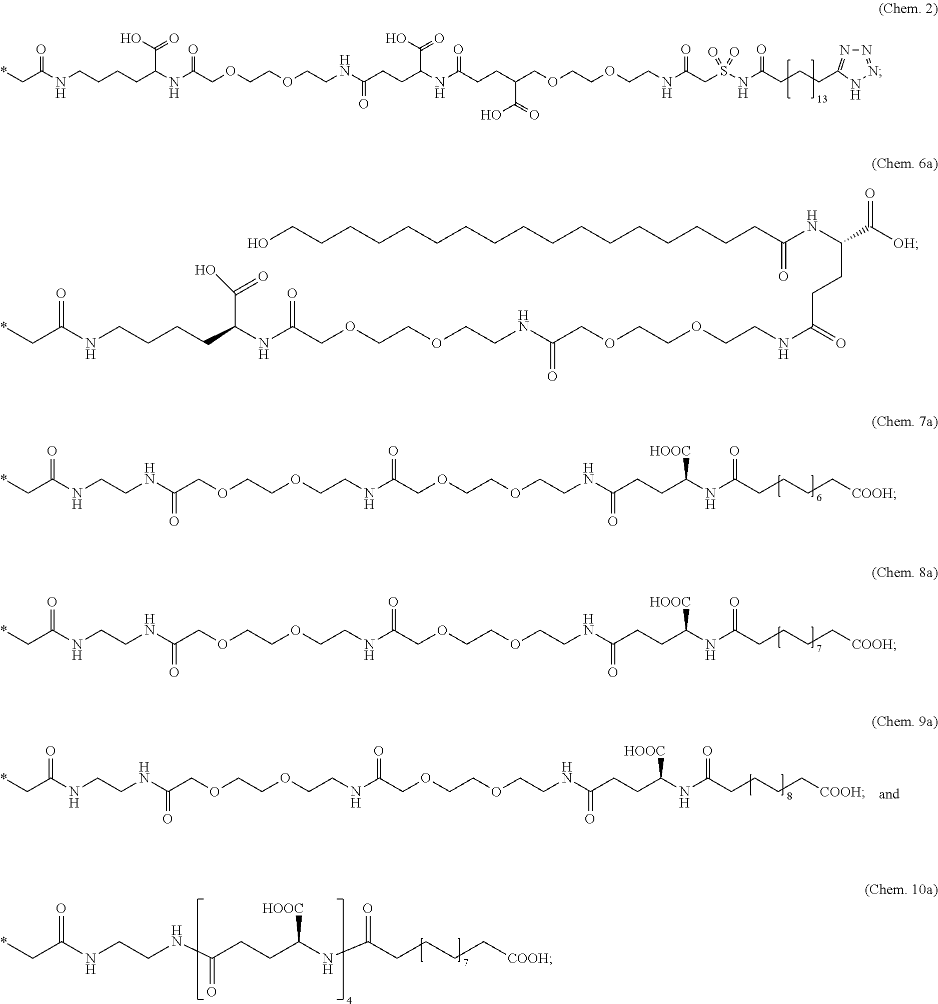
wherein * denotes the attachment point of the albumin binding moiety to the growth hormone variant via the sulphur residue of the cysteine side chain at position 101 of the growth hormone variant;
or a pharmaceutically acceptable salt thereof.

2. (canceled)

3. (canceled)

4. The long-acting growth hormone receptor antagonist according to claim 1, wherein the growth hormone variant comprises the following amino acid modifications compared to hGH:
H18D, H21N, L101C, G120R, R167N, K168A, D171S, K172R, E174S, and I179T (GHv-1, SEQ ID NO: 2);
H18D, H21N, L101C, G120R, N149D, N152D, R167N, K168A, D171S, K172R, E174S, and I179T (GHv-2, SEQ ID NO: 3); or
H18D, H21N, L101C, G120R, N149D, N152D, R167N, D171S, E174S, and I179T (GHv-3, SEQ ID NO: 4).

5. (canceled)

6. (canceled)

7. (canceled)

8. (canceled)

9. The long-acting growth hormone receptor antagonist according to claim 4, wherein the
albumin binding moiety is defined by Chem. 2 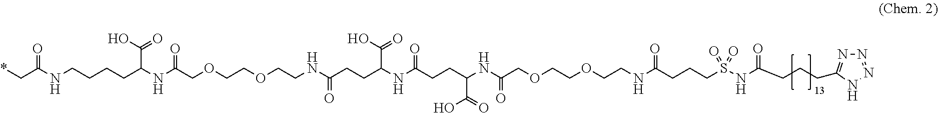
wherein * denotes the attachment point of the albumin binding moiety to the growth hormone variant via the sulphur residue of the cysteine side chain at position 101 of the growth hormone variant.

10. The long-acting growth hormone receptor antagonist according to claim 1, wherein the growth hormone variant comprises the following amino acid substitutions as compared to human growth hormone (hGH) (SEQ ID NO: 1): H18D, H21N, L101C, G120R, R167N, K168A, D171S, K172R, E174S, and I179T, and the albumin binding moiety is defined by Chem. 2

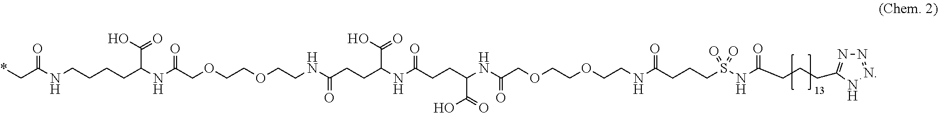

11. (canceled)

12. The long-acting growth hormone receptor antagonist according to claim 4, wherein the growth hormone variant consists of [H18D, H21N, L101C, G120R, R167N, K168A, D171S, K172R, E174S, 1179T]-hGH (SEQ ID NO: 2), and the albumin binding moiety is Chem. 2.

13. (canceled)

14. A long-acting growth hormone receptor antagonist selected from the group consisting of:

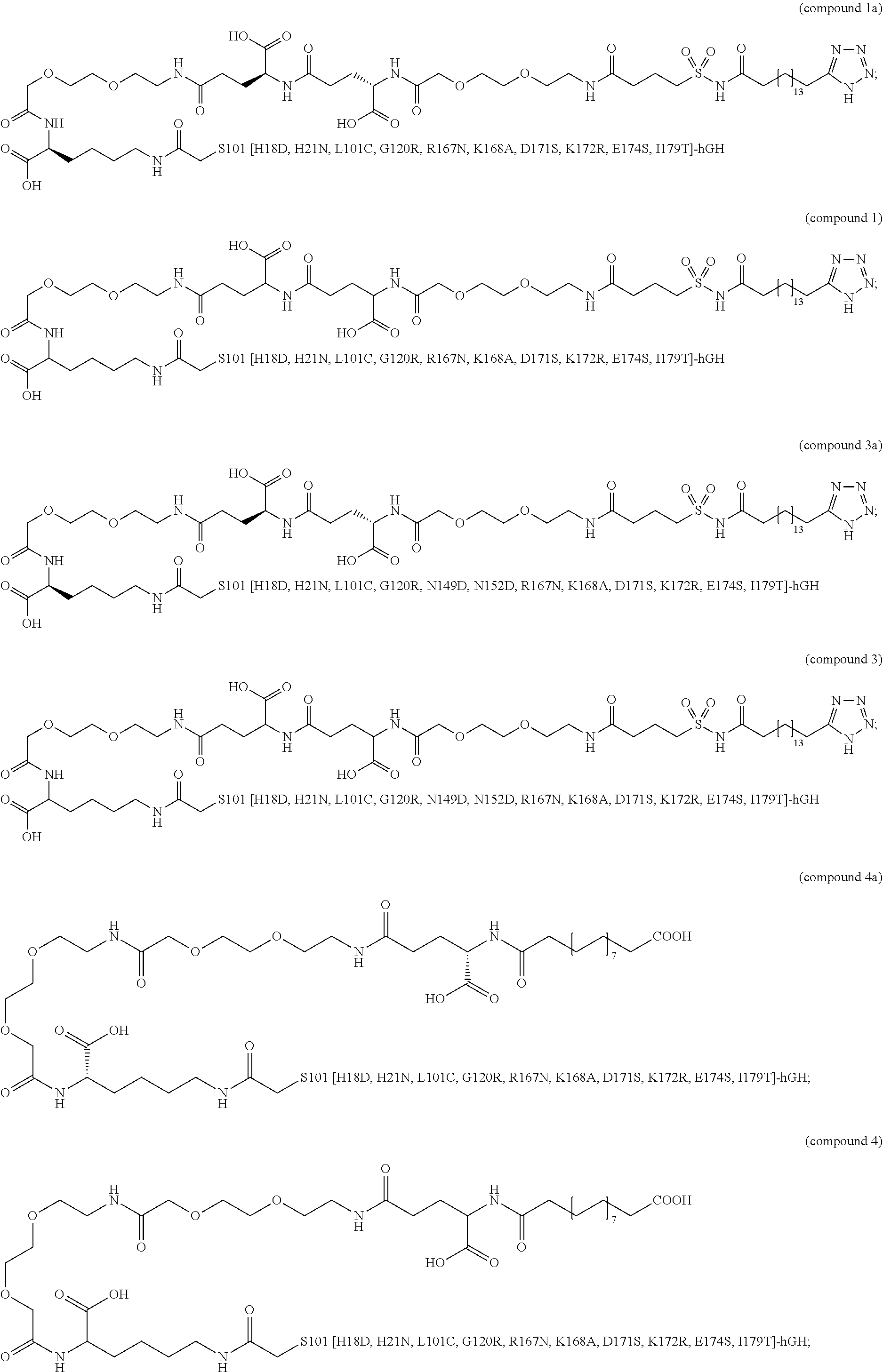

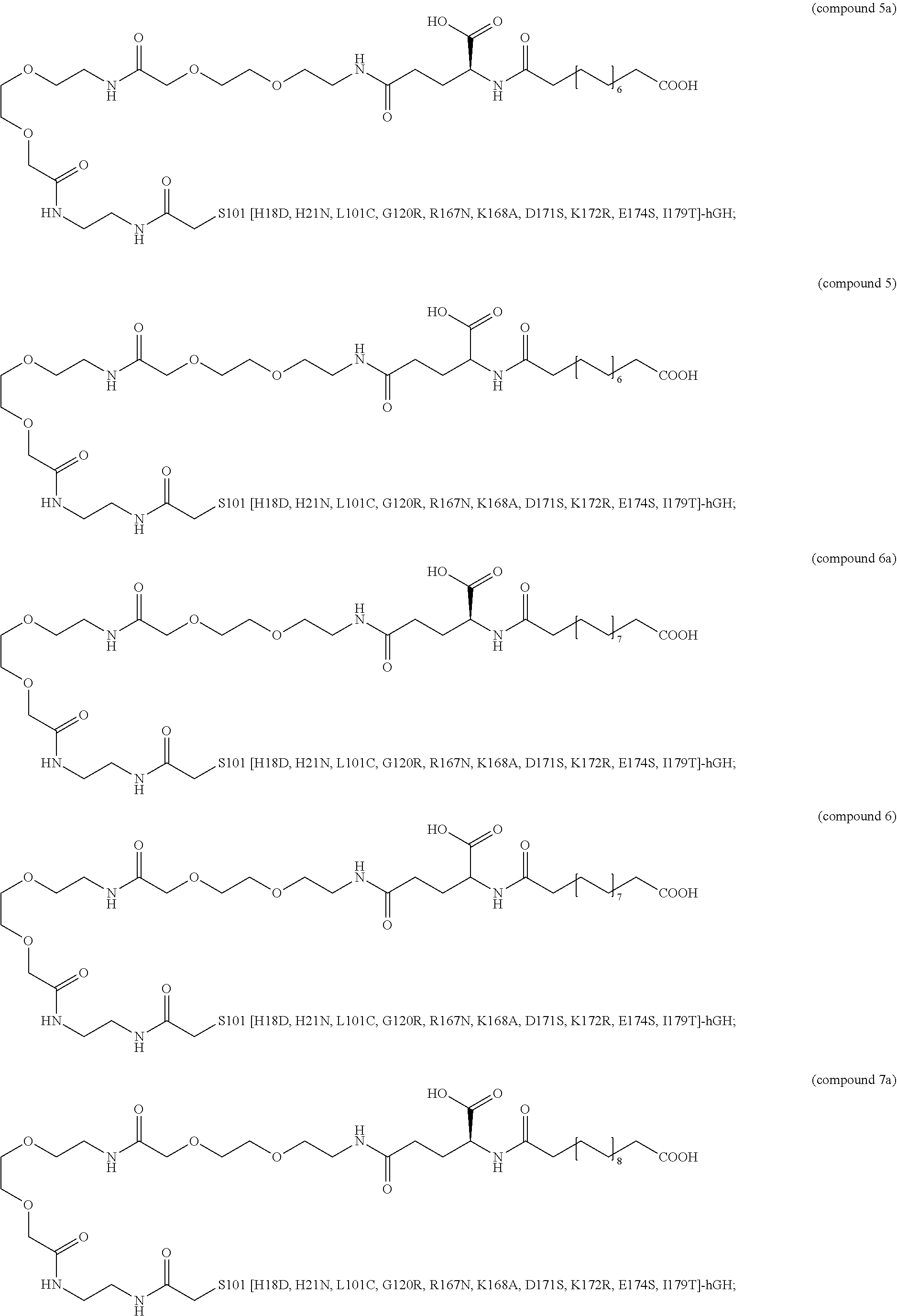

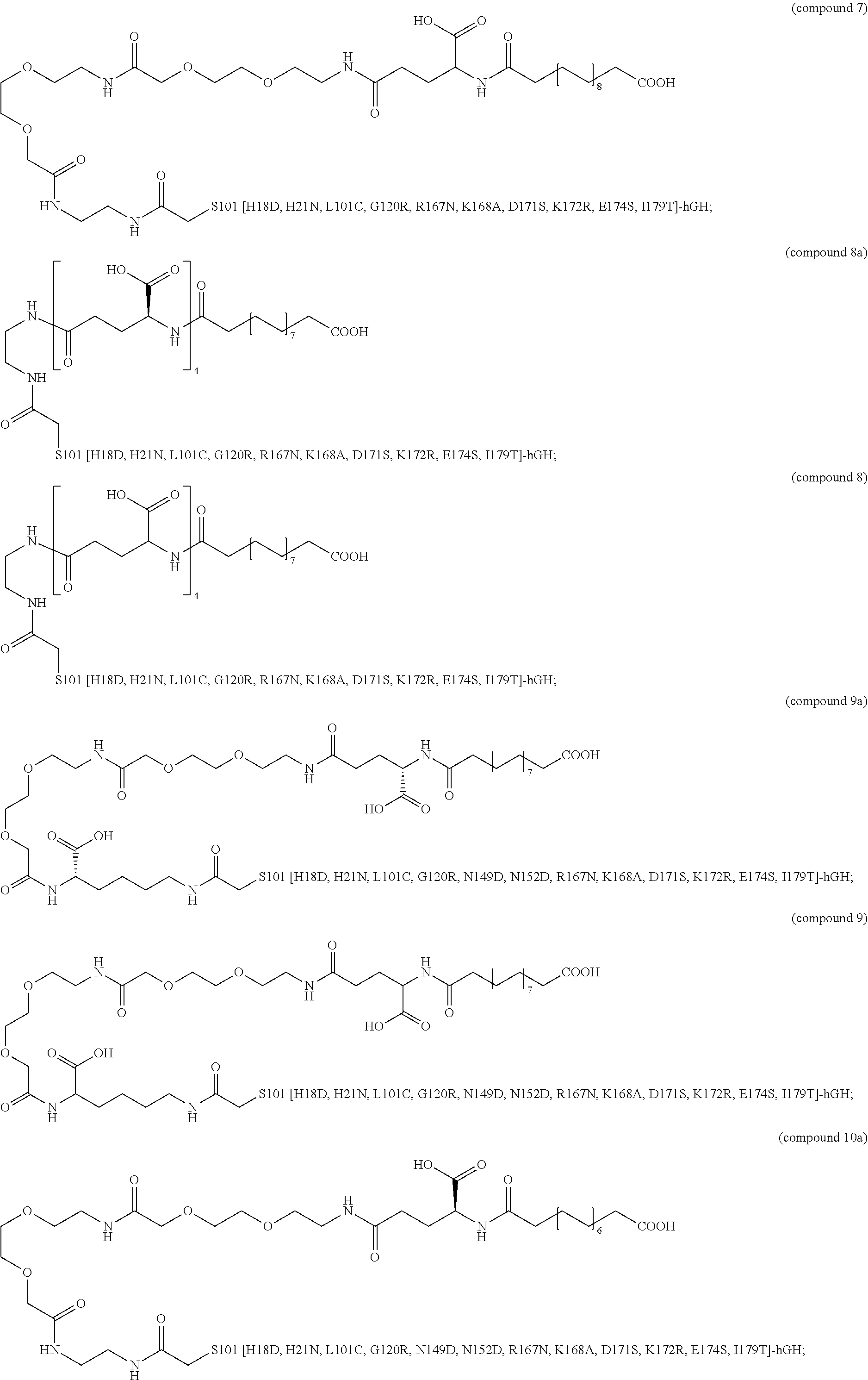

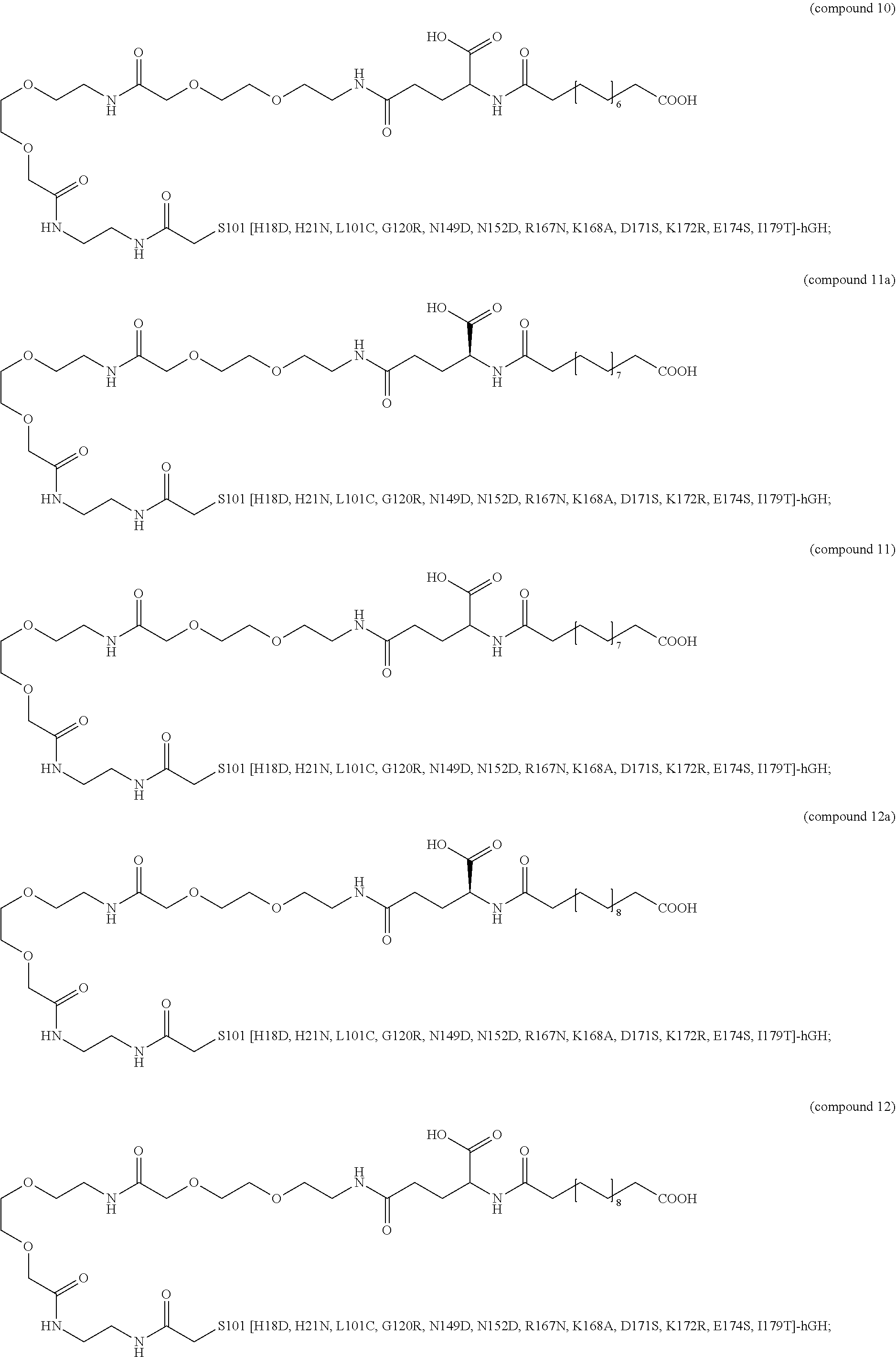

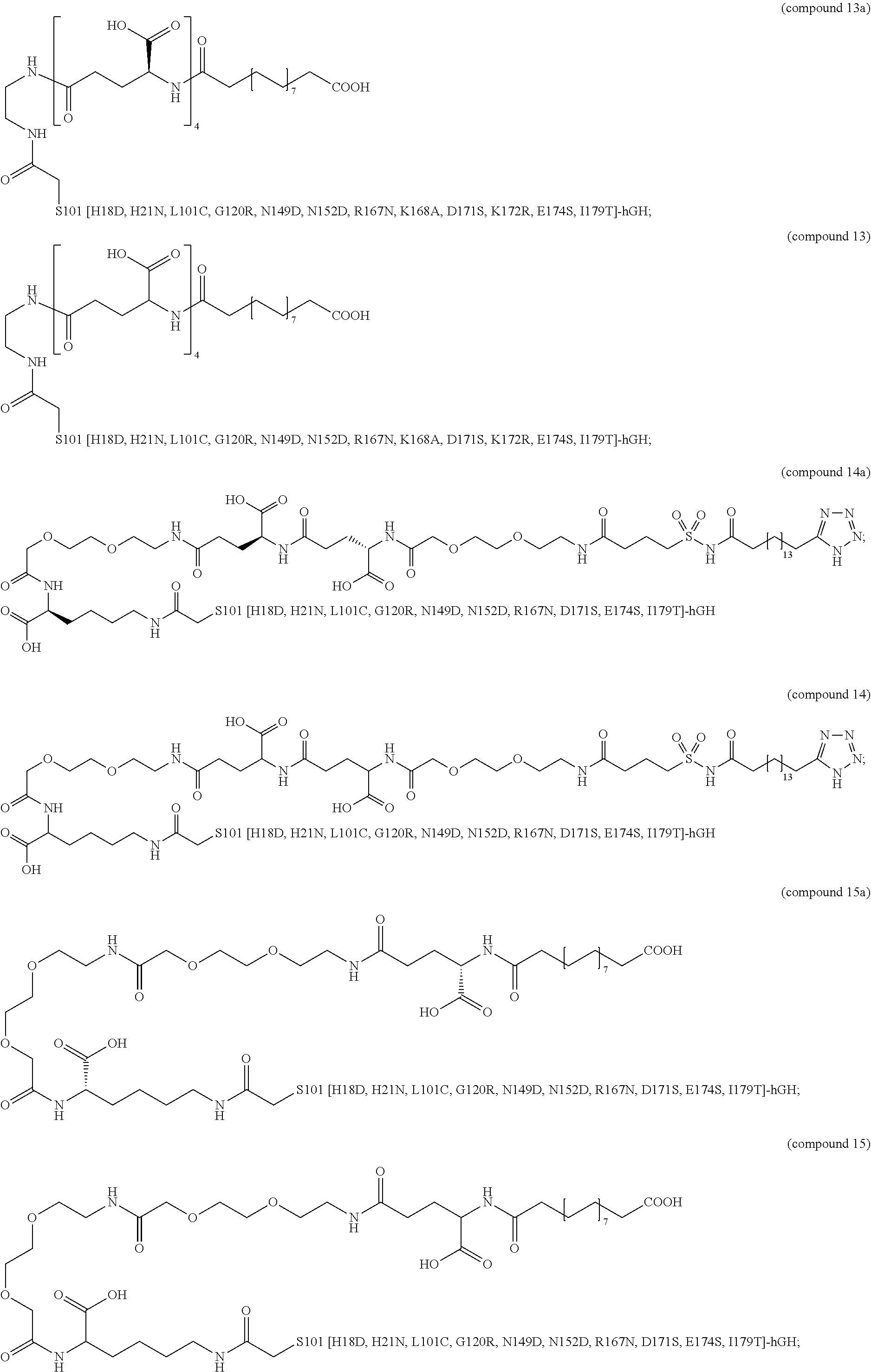

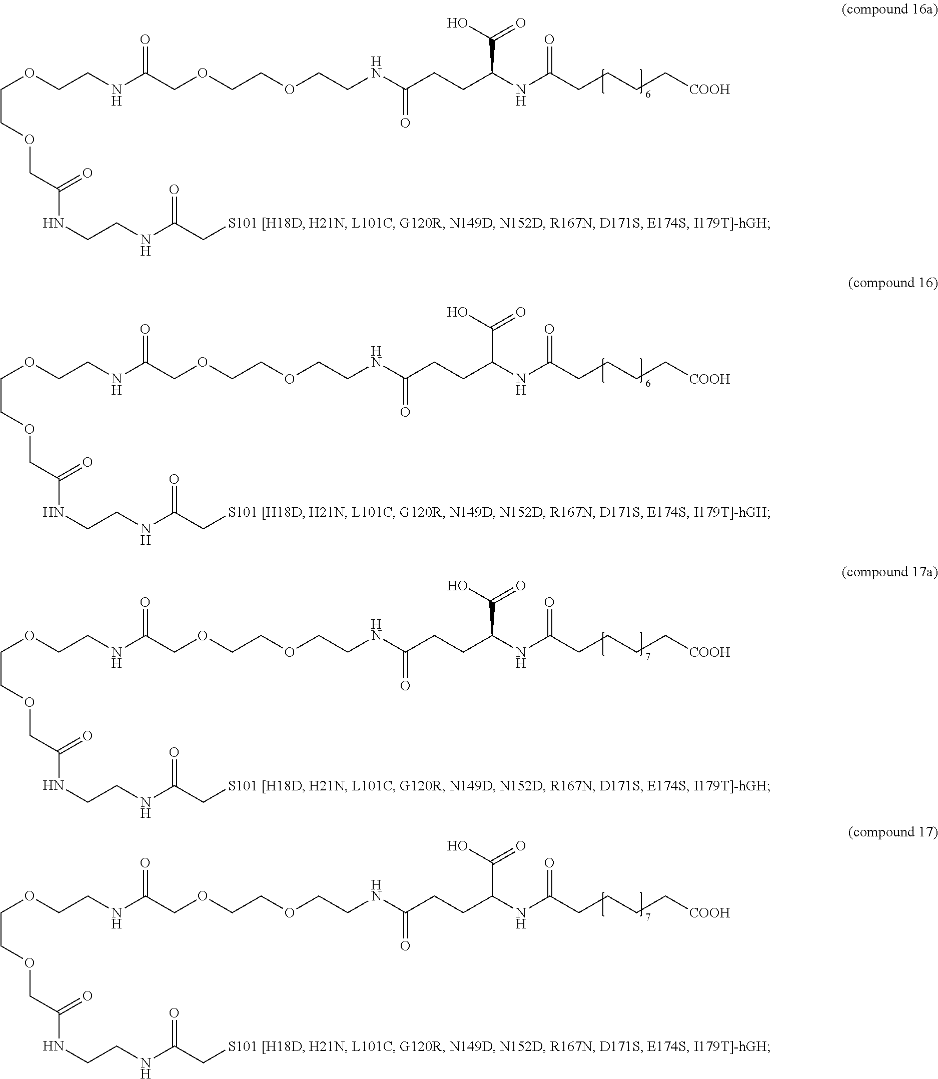

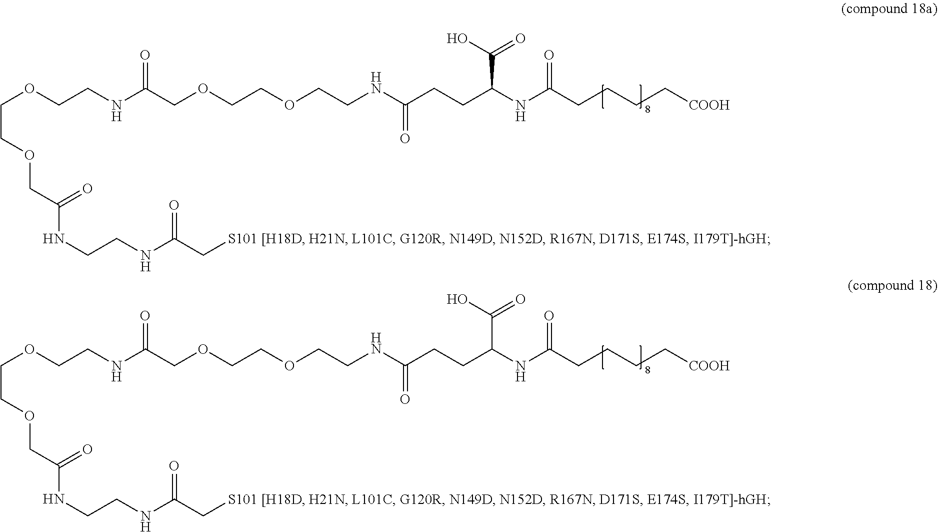

15. (canceled)

16. A long-acting growth hormone receptor antagonist of compound 1a: 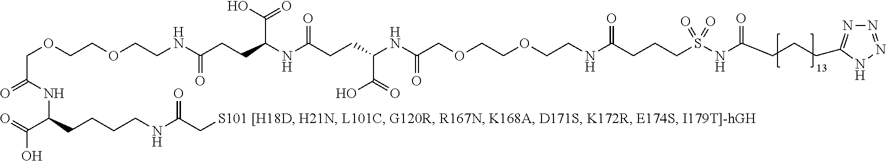
(compound 1a), or a pharmaceutically acceptable salt thereof, optionally having one or two disulphide bridges between Cys53-Cys165 and/or Cys182-Cys189.

17. (canceled)

18. The long-acting growth hormone receptor antagonist according to claim 16, wherein the growth hormone variant comprises two disulphide bridges between Cys53-Cys165 and Cys182-Cys189.

19. The long-acting growth hormone receptor antagonist according to claim 16, wherein the growth hormone variant comprises one a disulphide bridge between Cys53-Cys165.

20. The long-acting growth hormone receptor antagonist according to claim 16, wherein the growth hormone variant comprises one a disulphide bridge between Cys182-Cys189.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A pharmaceutical composition comprising the long-acting growth hormone receptor antagonist according to claim 14, and a pharmaceutically acceptable excipient.

33. The pharmaceutical composition according to claim 32, wherein the long-acting growth hormone receptor antagonist is compound 1 or compound 1a.

34. (canceled)

35. A method of treating a disease caused by excess human growth hormone and/or IGF-1 levels, comprising administering a pharmaceutical composition according to claim 33 to a patient in need thereof.

36. A nucleic acid encoding a human growth hormone variant according to claim 4.

37. A The nucleic acid according to claim 36, wherein said nucleic acid encodes a human growth hormone variant of GHv-1 (SEQ ID NO:2).

38. A vector comprising a nucleic acid according to claim 36.

39. A vector comprising a nucleic acid according to claim 37.

40. (canceled)

41. (canceled)

42. A host cell comprising a vector according to claim 38.

43. A process for preparing a human growth hormone variant selected from the group consisting of SEQ ID NO: 2, SEQ ID NO: 3, and SEQ ID NO: 4 comprising culturing a host cell according to claim 42.

44. (canceled)

45. The method according to claim 35, wherein the disease is acromegaly or gigantism.


SEQUENCE LISTING (5 sequences)

SEQ ID NO: 1 - protein, 191 aa, Homo sapiens [sequence omitted]

SEQ ID NO: 2 - protein, 191 aa, synthetic construct
FPTIPLSRLFDNAMLRADRLNQLAFDTYQEFEEAYIPKEQKYSFLQNPQTSLCFSESIPTPSNREETQQKSNLELLRISLLLIQSWLEPVQFLRSVFANSCVYGASDSNVYDLLKDLEERIQTLMGRLEDGSPRTGQIFKQTYSKFDTNSHNDDALLKNYGLLYCFNADMSRVSTFLRTVQCRSVEGSCGF

SEQ ID NO: 3 - protein, 191 aa, synthetic construct
FPTIPLSRLFDNAMLRADRLNQLAFDTYQEFEEAYIPKEQKYSFLQNPQTSLCFSESIPTPSNREETQQKSNLELLRISLLLIQSWLEPVQFLRSVFANSCVYGASDSNVYDLLKDLEERIQTLMGRLEDGSPRTGQIFKQTYSKFDTDSHDDDALLKNYGLLYCFNADMSRVSTFLRTVQCRSVEGSCGF

SEQ ID NO: 4 - protein, 191 aa, synthetic construct
FPTIPLSRLFDNAMLRADRLNQLAFDTYQEFEEAYIPKEQKYSFLQNPQTSLCFSESIPTPSNREETQQKSNLELLRISLLLIQSWLEPVQFLRSVFANSCVYGASDSNVYDLLKDLEERIQTLMGRLEDGSPRTGQIFKQTYSKFDTDSHDDDALLKNYGLLYCFNKDMSKVSTFLRTVQCRSVEGSCGF

SEQ ID NO: 5 - protein, 191 aa, synthetic construct
FPTIPLSRLFDNAMLRADRLNQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFNADMSRVSTFLRTVQCRSVEGSCGF